SEQUENCE LISTING (35 sequences)

SEQ ID NO: 1 - protein, 33 aa, synthetic construct
MEEAILVPCVLGLLLLPILAMLMALCVHCHRLP

SEQ ID NO: 2 - protein, 33 aa, synthetic construct
MEEAILVPCVLGLLLLPILAMLMALAVHCHRLP

SEQ ID NO: 3 - protein, 27 aa, synthetic construct
MEEAILVPCVLGLAMLMALCVHCHRLP

SEQ ID NO: 4 - protein, 33 aa, synthetic construct
MEEAILVLLLLLLLLLPLLLLLLLLCVHCHRLP

SEQ ID NO: 5 - protein, 106 aa, synthetic construct
MGSTRTEIIRELERSLREQRVLAIFLLALLIVLLWLLQQLKELLRELERLQREGSSDEDVRELLREIKELVENIVYLVIIIMVLVLVIIALARTQKYLVEELKRQD

SEQ ID NO: 6 - protein, 106 aa, synthetic construct
MGSTRTEIITRLSFSLLLQLVLAIFLLALLIVLLWLLQQLKELLRELERLQREGSSDEDVRELLREIKELVENIVYLVIIIMVLVLVIIALAVLQMYLVRELKRQD

SEQ ID NO: 7 - protein, 106 aa, synthetic construct
MGSTRTEIITRLSFSLLLQLVLAIFLLALLIVLLVLLIYLKELLRELERLQREGSSDEDVRELLREIKWLVIVIVALVIIIMVLVLVIIALAVLQMYLVRELKRQD

SEQ ID NO: 8 - protein, 73 aa, synthetic construct
MTENEIRKLRKLLRIAMFLLVFLLIWTWISLETSKTDDDPSAQSEALVAMSLMLIAASLLIIAKSKLMKSRNG

SEQ ID NO: 9 - protein, 73 aa, synthetic construct
MTKKIIMVLILLLIIAMLLLVFLLIATVVSLWWSWTDDDPSAISEALVAMSLMLIAASLLIIAISKLLKSKNG

SEQ ID NO: 10 - protein, 24 aa, synthetic construct
MGSSKSKPKDPSQRRNNNNGPVAT

SEQ ID NO: 11 - protein, 34 aa, synthetic construct
MGCIKSKRKDKDLELKLRILQSTVPRARDPPVAT

SEQ ID NO: 12 - protein, 19 aa, synthetic construct
FRSDGKKKKKKSKTKCQLL

SEQ ID NO: 13 - protein, 7 aa, synthetic construct
CKCCSIM

SEQ ID NO: 14 - DNA, 1083 bp, synthetic construct
  source 1..1083: mol_type = other DNA
atggaggaggccatcctggtcccctgcgtgctggggctcctgctgctgcccatcctggccatgttgatggcactgtgtgtgcactgccacagactgccaggctccggatccggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccgaattcgattacaaggaccacgatggcgactataaggatcacgacatcgactacaaggacgatgacgacaagtga

SEQ ID NO: 15 - DNA, 1077 bp, synthetic construct
  source 1..1077: mol_type = other DNA
atggaggaggccatcctggtcccctgcgtgctggggctcctgctgctgcccatcctggccatgttgatggcactggccgtgcactgccacagactgccaggatccggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccgaattcgattacaaggaccacgatggcgactataaggatcacgacatcgactacaaggacgatgacgacaagtga

SEQ ID NO: 16 - DNA, 1059 bp, synthetic construct
  source 1..1059: mol_type = other DNA
atggaggaggccatcctggtcccctgcgtgctggggctcgccatgttgatggcactgtgtgtgcactgccacagactgccaggatccggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccgaattcgattacaaggaccacgatggcgactataaggatcacgacatcgactacaaggacgatgacgacaagtga

SEQ ID NO: 17 - DNA, 1077 bp, synthetic construct
  source 1..1077: mol_type = other DNA
atggaagaggccatcctggttctgctgctgctccttctgcttctgctgcctcttctcttgctcctcctgctgctgtgcgtgcactgtcacagattgcctggatccggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccgaattcgattacaaggaccacgatggcgactataaggatcacgacatcgactacaaggacgatgacgacaagtga

SEQ ID NO: 18 - DNA, 1296 bp, synthetic construct
  source 1..1296: mol_type = other DNA
atgggcagcaccagaaccgagatcatcagagagctggaaagaagcctgcgcgagcagagagtgctggccatttttctgctggccctgctgatcgtgctgctgtggctgctgcaacagctgaaagagctgctgagagaactggaacggctgcagagagagggcagctctgacgaagatgtgcgggaactgctgcgcgagatcaaagaactggtggaaaacatcgtgtacctggttatcatcatcatggtgctggtgctcgtgatcattgccctggccagaacacagaagtacctggtcgaggaactgaagcggcaggacggatccggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccgaattcgattacaaggaccacgatggcgactataaggatcacgacatcgactacaaggacgatgacgacaagtga

SEQ ID NO: 19 - DNA, 1092 bp, synthetic construct
  source 1..1092: mol_type = other DNA
atgggcagcaccagaaccgagatcatcacccggctgagcttcagcctgctgctgcaactggtgctggctatctttctgctggccctgctgatcgtgctgctgtggctgcttcagcagctgaaagagctgctgagagagctggaacggctgcagagagagggaagctctgacgaggatgtgcgggaactgctgcgcgagatcaaagaactggtggaaaacatcgtgtacctggttatcatcatcatggtgctggtgctcgtgatcattgccctggccgtgctgcagatgtacctcgtcagggaactgaagcggcaggacggatccggcggctccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctccggctccgaattcgattacaaggaccacgatggcgactataaggatcacgacatcgactacaaggacgatgacgacaagtga

SEQ ID NO: 20 - DNA, 1092 bp, synthetic construct
  source 1..1092: mol_type = other DNA
atgggcagcaccagaaccgagatcatcacccggctgagcttcagcctgctgctgcaactggtgctggctatctttctgctggccctgctgatcgtgctgctggtgctcctgatctacctgaaagagctgctgagagagctggaacggctgcagagagagggaagctctgacgaggatgtgcgagaactgctgcgcgagatcaagtggctggtcatcgtgatcgtggccctggttatcatcatcatggtgctggtcctggtcatcattgccctggccgtgctgcagatgtacctcgtgcgggaactgaagagacaggacggatccggcggctccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctccggctccgaattcgattacaaggaccacgatggcgactataaggatcacgacatcgactacaaggacgatgacgacaagtga

SEQ ID NO: 21 - DNA, 1197 bp, synthetic construct
  source 1..1197: mol_type = other DNA
atgaccgagaacgagatccggaagctgagaaagctgctgcggatcgctatgttcctgctggtgtttctgctgatctggacctggatcagcctggaaaccagcaagaccgacgacgaccctagcgctcagtctgaagctctggtggccatgagcctgatgctgattgccgccagcctgctgatcattgccaagagcaagctgatgaagtcccggaacggaggatccggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccgaattcgattacaaggaccacgatggcgactataaggatcacgacatcgactacaaggacgatgacgacaagtga

SEQ ID NO: 22 - DNA, 993 bp, synthetic construct
  source 1..993: mol_type = other DNA
atgaccaagaaaatcatcatggtgctgatcctgctgctgatcattgctatgctgctgctggtgttcctgctgatcgccaccgtggtttccctgtggtggtcctggaccgacgatgatcctagcgccatttctgaagccctggtggccatgagcctgatgctgattgctgccagcctgctgattatcgccatcagcaagctgctgaagtccaagaacggcggatccggcggctccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctccggctccgaattcgattacaaggaccacgatggcgactataaggatcacgacatcgactacaaggacgatgacgacaagtga

SEQ ID NO: 23 - DNA, 939 bp, synthetic construct
  source 1..939: mol_type = other DNA
atggaattcggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccggatccgactacaaggacgatgacgacaagtga

SEQ ID NO: 24 - DNA, 1008 bp, synthetic construct
  source 1..1008: mol_type = other DNA
atgggcagttctaagagcaagcccaaggatcccagccagcggcggaacaacaacaatggacctgtggccactgaattcggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccggatccgactacaaggacgatgacgacaagtga

SEQ ID NO: 25 - DNA, 1038 bp, synthetic construct
  source 1..1038: mol_type = other DNA
atgggctgcatcaagagcaagcggaaggacaaggacctggaactgaagctgcggatcctgcagagcaccgtgcctagagctagagatcctccagtggccacagaattcggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccggatccgactacaaggacgatgacgacaagtga

SEQ ID NO: 26 - DNA, 1005 bp, synthetic construct
  source 1..1005: mol_type = other DNA
atggactacaaggacgatgacgacaaggaattcggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccggatccggtggtagctttcgtagtgatggcaaaaagaaaaagaagaaatccaagaccaaatgccagctgctgtga

SEQ ID NO: 27 - DNA, 984 bp, synthetic construct
  source 1..984: mol_type = other DNA
atggactacaaggacgatgacgacaaggaattcggcggctccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggctccggatccggtggtagcatgaaaaagcatcgttgtaaatgttgcagcattatgtga

SEQ ID NO: 28 - DNA, 1193 bp, synthetic construct
  source 1..1193: mol_type = other DNA
tggaattcggcggctcccctaagaaaaagcgcaaagtctccggaagccagtacctgcctgacaccgacgaccggcacagaatcgaggaaaagcggaagcggacctacgagacattcaagagcatcatgaagaagtccccattcagcggccccaccgatcctagacctccacctagaagaatcgccgtgcctagcagatccagcgcctctgtgcctaaacctgctcctcagccttatcctttcaccagcagcctgagcaccatcaactacgacgagttccccacaatggtgttccccagcggacagatcagccaggcttctgctcttgctccagctcctcctcaggttctgcctcaagctcctgctccggctccagcaccagctatggtttctgctttggcccaggctcctgcacctgtgcctgttcttgctcctggaccacctcaggctgttgctccaccagctcctaaacctacacaggccggcgagggaacactgtctgaagccctgctgcaactccagttcgacgacgaggatctgggagcactgctgggcaatagcacagaccctgccgtgtttaccgatctggccagcgtggacaacagcgagtttcagcagctcctgaaccagggcatccctgtggctcctcacaccacagagcccatgctgatggaataccccgaggccatcaccagactggtcaccggcgctcaaagacctccagatcctgcaccagcacctcttggagcacctggcctgcctaatggactgctgagcggagatgaggacttcagctctatcgccgacatggatttcagcgccctgctcggttccggtggtggcgggtctggaggaggaggtagtggtggaggtggctctggtaccgctagacccggcgaaagacctttccagtgccggatctgcatgaggaacttcagcaagggcgagagactcgtgcggcacaccagaacacacacaggcgagaagcccttccagtgtagaatctgtatgcgcaacttcagccggatggacaacctgagcacccacctgagaacccataccggggagaagccatttcaatgccgcatctgtatgagaaatttttcccggaaggacgccctgaaccggcacctgaaaacacacctgagaggcagcggctccggatccgactacaaggacgatgacgacaagtga

SEQ ID NO: 29 - DNA, 1263 bp, synthetic construct
  source 1..1263: mol_type = other DNA
atgggcagttctaagagcaagcccaaggaccctagccagcggcggaacaacaacaatggacctgtggccactgaattcggcggctcccctaagaaaaagcgcaaagtctccggaagccagtacctgcctgacaccgacgaccggcacagaatcgaggaaaagcggaagcggacctacgagacattcaagagcatcatgaagaagtccccattcagcggccccaccgatcctagacctccacctagaagaatcgccgtgcctagcagatccagcgcctctgtgcctaaacctgctcctcagccttatcctttcaccagcagcctgagcaccatcaactacgacgagttccccacaatggtgttccccagcggacagatcagccaggcttctgctcttgctccagctcctcctcaggttctgcctcaagctcctgctccggctccagcaccagctatggtttctgctttggcccaggctcctgcacctgtgcctgttcttgctcctggaccacctcaggctgttgctccaccagctcctaaacctacacaggccggcgagggaacactgtctgaagccctgctgcaactccagttcgacgacgaggatctgggagcactgctgggcaatagcacagaccctgccgtgtttaccgatctggccagcgtggacaacagcgagtttcagcagctcctgaaccagggcatccctgtggctcctcacaccacagagcccatgctgatggaataccccgaggccatcaccagactggtcaccggcgctcaaagacctccagatcctgcaccagcacctcttggagcacctggcctgcctaatggactgctgagcggagatgaggacttcagctctatcgccgacatggatttcagcgccctgctcggttccggtggtggcgggtctggaggaggaggtagtggtggaggtggctctggtaccgctagacccggcgaaagacctttccagtgccggatctgcatgaggaacttcagcaagggcgagagactcgtgcggcacaccagaacacacacaggcgagaagcccttccagtgtagaatctgtatgcgcaacttcagccggatggacaacctgagcacccacctgagaacccataccggggagaagccatttcaatgccgcatctgtatgagaaatttttcccggaaggacgccctgaaccggcacctgaaaacacacctgagaggcagcggctccggatccgactacaaggacgatgacgacaagtga

SEQ ID NO: 30 - DNA, 1293 bp, synthetic construct
  source 1..1293: mol_type = other DNA
atgggctgcatcaagagcaagcggaaggacaaggacctggaactgaagctgagaatcctgcagagcaccgtgcctagagctagagatcctccagtggccacagaattcggcggctcccctaagaaaaagcgcaaagtctccggaagccagtacctgcctgacaccgacgaccggcacagaatcgaggaaaagcggaagcggacctacgagacattcaagagcatcatgaagaagtccccattcagcggccccaccgatcctagacctccacctagaagaatcgccgtgcctagcagatccagcgcctctgtgcctaaacctgctcctcagccttatcctttcaccagcagcctgagcaccatcaactacgacgagttccccacaatggtgttccccagcggacagatcagccaggcttctgctcttgctccagctcctcctcaggttctgcctcaagctcctgctccggctccagcaccagctatggtttctgctttggcccaggctcctgcacctgtgcctgttcttgctcctggaccacctcaggctgttgctccaccagctcctaaacctacacaggccggcgagggaacactgtctgaagccctgctgcaactccagttcgacgacgaggatctgggagcactgctgggcaatagcacagaccctgccgtgtttaccgatctggccagcgtggacaacagcgagtttcagcagctcctgaaccagggcatccctgtggctcctcacaccacagagcccatgctgatggaataccccgaggccatcaccagactggtcaccggcgctcaaagacctccagatcctgcaccagcacctcttggagcacctggcctgcctaatggactgctgagcggagatgaggacttcagctctatcgccgacatggatttcagcgccctgctcggttccggtggtggcgggtctggaggaggaggtagtggtggaggtggctctggtaccgctagacccggcgaaagacctttccagtgccggatctgcatgaggaacttcagcaagggcgagagactcgtgcggcacaccagaacacacacaggcgagaagcccttccagtgtagaatctgtatgcgcaacttcagccggatggacaacctgagcacccacctgagaacccataccggggagaagccatttcaatgccgcatctgtatgagaaatttttcccggaaggacgccctgaaccggcacctgaaaacacacctgagaggcagcggctccggatccgactacaaggacgatgacgacaagtga

SEQ ID NO: 31 - DNA, 1260 bp, synthetic construct
  source 1..1260: mol_type = other DNA
atggactacaaggacgatgacgacaaggaattcggcggctcccctaagaaaaagcgcaaagtctccggaagccagtacctgcctgacaccgacgaccggcacagaatcgaggaaaagcggaagcggacctacgagacattcaagagcatcatgaagaagtccccattcagcggccccaccgatcctagacctccacctagaagaatcgccgtgcctagcagatccagcgcctctgtgcctaaacctgctcctcagccttatcctttcaccagcagcctgagcaccatcaactacgacgagttccccacaatggtgttccccagcggacagatcagccaggcttctgctcttgctccagctcctcctcaggttctgcctcaagctcctgctccggctccagcaccagctatggtttctgctttggcccaggctcctgcacctgtgcctgttcttgctcctggaccacctcaggctgttgctccaccagctcctaaacctacacaggccggcgagggaacactgtctgaagccctgctgcaactccagttcgacgacgaggatctgggagcactgctgggcaatagcacagaccctgccgtgtttaccgatctggccagcgtggacaacagcgagtttcagcagctcctgaaccagggcatccctgtggctcctcacaccacagagcccatgctgatggaataccccgaggccatcaccagactggtcaccggcgctcaaagacctccagatcctgcaccagcacctcttggagcacctggcctgcctaatggactgctgagcggagatgaggacttcagctctatcgccgacatggatttcagcgccctgctcggttccggtggtggcgggtctggaggaggaggtagtggtggaggtggctctggtaccgctagacccggcgaaagacctttccagtgccggatctgcatgaggaacttcagcaagggcgagagactcgtgcggcacaccagaacacacacaggcgagaagcccttccagtgtagaatctgtatgcgcaacttcagccggatggacaacctgagcacccacctgagaacccataccggggagaagccatttcaatgccgcatctgtatgagaaatttttcccggaaggacgccctgaaccggcacctgaaaacacacctgagaggcagcggctccggatccggtggtagctttcgtagtgatggcaaaaagaaaaagaagaaatccaagaccaaatgccagctgctgtga

SEQ ID NO: 32 - DNA, 1239 bp, synthetic construct
  source 1..1239: mol_type = other DNA
atggactacaaggacgatgacgacaaggaattcggcggctcccctaagaaaaagcgcaaagtctccggaagccagtacctgcctgacaccgacgaccggcacagaatcgaggaaaagcggaagcggacctacgagacattcaagagcatcatgaagaagtccccattcagcggccccaccgatcctagacctccacctagaagaatcgccgtgcctagcagatccagcgcctctgtgcctaaacctgctcctcagccttatcctttcaccagcagcctgagcaccatcaactacgacgagttccccacaatggtgttccccagcggacagatcagccaggcttctgctcttgctccagctcctcctcaggttctgcctcaagctcctgctccggctccagcaccagctatggtttctgctttggcccaggctcctgcacctgtgcctgttcttgctcctggaccacctcaggctgttgctccaccagctcctaaacctacacaggccggcgagggaacactgtctgaagccctgctgcaactccagttcgacgacgaggatctgggagcactgctgggcaatagcacagaccctgccgtgtttaccgatctggccagcgtggacaacagcgagtttcagcagctcctgaaccagggcatccctgtggctcctcacaccacagagcccatgctgatggaataccccgaggccatcaccagactggtcaccggcgctcaaagacctccagatcctgcaccagcacctcttggagcacctggcctgcctaatggactgctgagcggagatgaggacttcagctctatcgccgacatggatttcagcgccctgctcggttccggtggtggcgggtctggaggaggaggtagtggtggaggtggctctggtaccgctagacccggcgaaagacctttccagtgccggatctgcatgaggaacttcagcaagggcgagagactcgtgcggcacaccagaacacacacaggcgagaagcccttccagtgtagaatctgtatgcgcaacttcagccggatggacaacctgagcacccacctgagaacccataccggggagaagccatttcaatgccgcatctgtatgagaaatttttcccggaaggacgccctgaaccggcacctgaaaacacacctgagaggcagcggctccggatccggtggtagcatgaaaaagcatcgttgtaaatgttgcagcattatgtga

SEQ ID NO: 33 - protein, 295 aa, synthetic construct
EIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 34 - protein, 225 aa, synthetic construct
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 35 - protein, 381 aa, synthetic construct
PKKKRKVSGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGGGGSGGGGSGGGGSGTARPGERPFQCRICMRNFSKGERLVRHTRTHTGEKPFQCRICMRNFSRMDNLSTHLRTHTGEKPFQCRICMRNFSRKDALNRHLKTHLRGSGS

(19) United States
(12) Patent Application Publication
(10) Pub. No.: US 2026/0306415 A1
(43) Pub. Date: Oct. 8, 2026

(54) ACTIVE LOADING OF BIOMOLECULAR CARGO INTO ENGINEERED VESICLES

(71) Applicant: NORTHWESTERN UNIVERSITY, Evanston, IL (US)

(72) Inventors: Justin Alexander Peruzzi, Evanston, IL (US); Taylor Franklin Gunnels, Evanston, IL (US); Neha Prashant Kamat, Evanston, IL (US); Joshua N. Leonard, Evanston, IL (US)

(73) Assignee: Northwestern University, Evanston, IL (US)

(21) Appl. No.: 19/478,352

(22) PCT Filed: Apr. 25, 2024
(86) PCT No.: PCT/US2024/026170

§ 371 Date: Oct. 24, 2025

Related U.S. Application Data
(60) Provisional application No. 63/462,120, filed on Apr. 26, 2023.

(51) Int. Cl. A61K 9/50; A61K 38/00; C07K 14/47; C12N 15/62; C12N 15/88

(52) U.S. Cl. CPC A61K 9/5068; A61K 9/5089; C07K 14/4702; C12N 15/62; C12N 15/88; A61K 38/00; C07K 2319/03

(57) ABSTRACT

The present disclosure provides, among other things, improved methods and compositions for loading an engineered vesicle (EngV) with a fusion polypeptide or biomolecular cargo, loaded EngVs or a plurality thereof (e.g., produced in accordance with the methods described herein), compositions comprising such loaded EngVs or a plurality thereof, and methods of treating a disease or disorder by administering compositions of the present disclosure.

### CROSS REFERENCE TO RELATED APPLICATIONS

This application claims the benefit of and priority to U.S. Provisional Patent Application No. 63/462,120, filed on Apr. 26, 2023, the contents of which are incorporated herein by reference in its entirety.

### GOVERNMENT LICENSING RIGHTS STATEMENT

This invention was made with Government support under Grant Nos. CBET-1844219 and CBET-1844336 awarded by the National Science Foundation (NSF). The government has certain rights in the invention.

### BACKGROUND

The following description of the background of the present technology is provided simply as an aid in understanding the present technology and is not admitted to describe or constitute prior art to the present technology.

Engineered vesicles (EngVs) are cell-derived, lipid enclosed particles. EngVs include, for example, exosomes and microvesicles, which are small lipid enclosed carriers of biomolecular cargos, such as polypeptides, lipids, and nucleic acids. EngVs can be secreted by cells and facilitate both intracellular communication as well as biomolecular cargo transport. Due to certain EngVs innate properties (e.g., intrinsic tissue-homing capabilities), EngVs have become highly considered for use in the delivery of therapeutic payloads as the biomolecular cargo (e.g., therapeutic polypeptides). However, there are a plurality of challenges in using EngVs as delivery vehicles, such as stably encapsulating or associating biomolecular cargo into or with EngVs (e.g., in therapeutically effective amounts), such as therapeutic polypeptides, without compromising the functional activity of the biomolecular cargo (e.g., by disrupting its structure and/or localization in the cell).

To advance technologies related to utilizing EngVs as a delivery modality, it is important to be able to efficiently load EngVs with a biomolecular cargo of interest (e.g., polypeptides, therapeutic polypeptides, nucleic acids, viral particles or cores) without compromising their functional activity. Accordingly, there remains a need for improved methods and compositions of loading (also referred to herein as “making”) an EngV with biomolecular cargo (e.g., therapeutic polypeptides).

### SUMMARY OF THE INVENTION

The present disclosure provides, among other things, improved compositions and methods for loading (making) an EngV with biomolecular cargo (e.g., fusion polypeptides comprising a biomolecular cargo as described herein).

In one aspect, the present disclosure provides a method of loading an engineered vesicle (EngV) with a fusion polypeptide, comprising: contacting a cell with a nucleic acid encoding a fusion polypeptide comprising (i) a biomolecular cargo, and (ii) one or more lipid tags, wherein contacting occurs under conditions sufficient to permit (i) uptake of the nucleic acid by the cell, (ii) translation of the fusion polypeptide, and (iii) post-translational modification of the one or more lipid tags with one or more lipids by endogenous host machinery in the cell to thereby generate a lipid-modified fusion polypeptide, and wherein the lipid-modified fusion polypeptide is directed and loaded into or on the EngV.

In some embodiments, the one or more lipid tags comprise a short amino acid sequence which a host cell can post-translationally modify with one or more lipids. In some embodiments, the one or more lipid tags comprises the amino acid sequence of any one of SEQ ID NOs: 1-13.

In some embodiments, post-translational modification of the one or more lipid tags with a lipid comprises one or more of acylation or prenylation. In some embodiments, acylation comprises myristoylation, palmitoylation, palmitoleoylation, stearoylation, or any combination thereof. In some embodiments, prenylation comprises farnesylation, geranylgeranylation, or any combination thereof.

In some embodiments, post-translational modification of the one or more lipid tags with a lipid comprises palmitoylation and myristoylation.

In some embodiments, post-translational modification of the one or more lipid tags with a lipid comprises palmitoylation and farnesylation.

In some embodiments, the lipid-modified fusion polypeptide is myristoylated, palmitoylated, palmitoleoylated, stearoylated, geranylgeranylated, farnesylated, or any combination thereof.

In some embodiments, the lipid-modified fusion polypeptide is myristoylated.

In some embodiments, the lipid-modified fusion polypeptide is palmitoylated and myristoylated.

In some embodiments, the lipid-modified fusion polypeptide is triple palmitoylated.

In some embodiments, the lipid-modified fusion polypeptide is geranylgeranylated.

In some embodiments, the lipid-modified fusion polypeptide is double palmitoylated and farnesylated.

In some embodiments, the biomolecular cargo is or comprises a therapeutic polypeptide. In some embodiments, the therapeutic polypeptide is a transcription factor.

In some embodiments, methods of the present disclosure further comprise isolating the loaded EngV

In some embodiments, methods of the present disclosure further comprise characterizing the loaded EngV for one or more of yield, purity, loading, functional activity, or any combination thereof.

In some embodiments, the biomolecular cargo comprises a transmembrane domain and the transmembrane domain is modified to enhance direction and loading into or on the EngV relative to the biomolecular cargo with an unmodified transmembrane domain. In some embodiments, the transmembrane domain is modified by one or more of: changing the length of the transmembrane domain and changing the transmembrane domain surface area.

In some embodiments, the present disclosure provides a nucleic acid encoding fusion polypeptides described herein.

In one aspect, the present disclosure provides a fusion polypeptide comprising (i) a biomolecular cargo and (ii) one or more lipid tags, wherein the one or more lipid tags comprise a short amino acid sequence which a host cell can post-translationally modify with one or more lipids.

In one aspect, the present disclosure provides a method of loading an engineered vesicle (EV) with a biomolecular cargo, comprising: contacting a cell with a nucleic acid encoding the biomolecular cargo, wherein contacting occurs under conditions sufficient to permit (i) uptake of the nucleic acid by the cell, (ii) translation of the biomolecular cargo, wherein the biomolecular cargo comprises a transmembrane domain and the transmembrane domain is modified to improve direction and loading into or on the EngV relative to the biomolecular cargo with an unmodified transmembrane domain, thereby generating a transmembrane modified biomolecular cargo, and wherein the transmembrane modified biomolecular cargo is directed and loaded into or on the EngV.

In some embodiments, the transmembrane domain is modified by one or more of: changing the length of the transmembrane domain and changing the transmembrane domain surface area.

In some embodiments, the transmembrane modified biomolecular cargo further comprises one or more lipid tags that can be post-translationally modified with one or more lipids by endogenous host machinery in the cell to thereby generate a lipid-modified, transmembrane modified biomolecular cargo, and wherein the lipid-modified, transmembrane modified biomolecular cargo is directed and loaded into or on the EngV.

In some embodiments, the present disclosure provides a loaded EngV or a plurality thereof prepared by a method described herein.

In some embodiments, the present disclosure provides a composition comprising the loaded EngV or a plurality thereof described herein. In some embodiments, the composition is a pharmaceutical composition.

In some embodiments, the present disclosure provides a method of treating a disease or a disorder in a subject in need thereof comprising administering the loaded EngV or a plurality thereof of the present disclosure to the subject in need thereof.

In some embodiments, the present disclosure provides a nucleic acid encoding a transmembrane modified biomolecular cargo. In some embodiments, the nucleic acid further comprises one or more lipid tags.

### BRIEF DESCRIPTION OF THE DRAWINGS

FIG. 1A-1C. shows that extracellular vesicle (EV) populations from HEK293FT cells demonstrate classical EV characteristics. FIG. 1A provides a cartoon depicting the process for isolating two EV subtypes, microvesicles (MV) and exosomes (Exo), from conditioned cell culture medium. FIG. 1B shows representative histogram of particle sizes from the EV subpopulations described in FIG. 1A as determined by nanoparticle tracking analysis. FIG. 1C shows Western blots on HEK293FT EV samples and cell lysates for EV markers CD9, CD81, and Alix and the endoplasmic reticulum-associated (non EV-associated) marker, Calnexin (n=1). Equal numbers of EVs were added for each blot. For the Calnexin blot, 4.3×10⁸ EVs were used per well, and 2.7 μg of lysate was added.

FIG. 2A-2D demonstrate that EV membrane physical properties and protein content mirror those of lipid rafts. FIG. 2A provides a schematic of the hypothesis that lipid-raft association could be used as a handle to load proteins into EVs. FIG. 2B shows the overall analysis and experimental workflow. RaftProt 2.0 and Exocarta were used to understand features of proteins found in EVs and lipid rafts (I); a library of structurally diverse proteins was built to understand how such features affect protein trafficking, interactions with lipid rafts, and loading into EVs (II); these design rules were applied, and demonstrated how lipid-protein interactions can be used to functionally deliver cargo to cells via EVs (III). FIG. 2C shows Laurdan generalized polarization (GP) of whole cells, ordered liposomes (L_{O}) composed of 70 mol % DPPC/30 mol % Chol, disordered liposomes (L_{D}) composed of 70 mol % DOPC/30 mol % Chol, HEK293FT cells, and microvesicles (MVs) and exosomes (Exos) derived from HEK293FT cells. MV and Exos were found to have high laurdan GP, similar to L_{O} membranes. Each dot represents an independent experiment. FIG. 2D shows the frequency that human membrane-associated proteins and raft proteins are found in EVs as calculated via bioinformatic analysis. Raft associated proteins are more frequently found in EVs compared to a random selection of human membrane-associated proteins. Each dot represents a separate query of 100 human proteins. n>3, error bars represent the standard error of the mean (SEM).

FIG. 3 shows Laurdan spectra of samples in FIG. 2C. This demonstrates that microvesicles (MV) and exosomes (Exo) are more similar in lipid order to ordered liposomes (L_{O}) than disordered liposomes (L_{D}). L_{O} were composed of 70 mol % DPPC/30 mol % Chol, and L_{D} were composed of 70 mol % DOPC/30 mol % Chol. Whole cells refers to HEK293FT cells; the EVs in this experiment were derived from HEK293FTs. Spectra were normalized to the maximum fluorescence. Samples were excited by a 350 nm laser and fluorescence intensities were collected from 400 to 600 nm. Intensities at 439 nm (blue line) and 483 nm (red line) were used to calculate Laurdan generalized polarization (GP) via the formula in the methods section. 1-3, error bars represent the SEM.

FIG. 4 shows Laurdan spectra of exosomes as a function of temperature and demonstrates a characteristic emission profile. Samples were excited by a 350 nm laser and fluorescence intensities were collected from 400 to 600 nm. Laurdan emission exhibits a slight red shift as temperature increases which reflects the expected decrease in membrane order as temperature increases. Spectra were normalized to the maximum fluorescence. n=2, error bars represent the SEM.

FIG. 5A-5G demonstrates that protein structural features contribute to raft and EV membrane association. Features of plasma membrane-associated proteins known to associate with lipid rafts and EVs were compared to all human plasma membrane proteins. Average transmembrane domain length and number of palmitoyls per protein (N_{palmitoyl}/protein) for (FIG. 5A, 5B) single-pass and (FIG. 5C, 5D) multi-pass transmembrane proteins, and (FIG. 5E, 5F, 5G) number of myristoyl, palmitoyl, and prenyl groups on peripheral membrane proteins were compared. Error bars represent SEM, number of proteins in each category can be found in Table 8. A one-way ANOVA was performed to compare structural features between each data set, and comparisons were evaluated using the Sidak multiple comparisons correction (*, p<0.05, **, p<0.01, ***, p<0.001).

FIG. 6A-6H shows that for single transmembrane domain proteins, transmembrane domain length and number of posttranslational modifications can vary between proteins found in lipid rafts (Raft, from Raftprot 2.0), EVs (Exo, from Exocarta), and all human membrane proteins (All, from Swiss-Prot). Specifically, FIG. 6A shows that transmembrane domain length and the average number of disulfides (FIG. 6B), phosphoryl groups (FIG. 6C), sugars (glycosylation) (FIG. 6D), palmitoyls (FIG. 6E), myristoyls (FIG. 6F), prenyls (FIG. 6G), and GPI anchors (FIG. 6H) on each protein were calculated. FIG. 6G shows no prenylation of single transmembrane proteins was observed. Proteins were separated by the membrane which they localize to: the plasma membrane (PM) or internal membranes (IM). Error bars represent the SEM.

FIG. 7A-7H shows that for multi-transmembrane domain proteins, transmembrane domain length and number of posttranslational modifications can vary between proteins found in lipid rafts (Raft, from Raftprot 2.0), EVs (Exo, from Exocarta), and all human membrane proteins (All, from Swiss-Prot). Specifically, transmembrane domain length (FIG. 7A) and the average number of disulfides (FIG. 7B), phosphoryl groups (FIG. 7C), d sugars (glycosylation) (FIG. 7D), palmitoyls (FIG. 7E), myristoyls (FIG. 7F), prenyls (FIG. 7G), and GPI anchors (FIG. 7H) on each protein were calculated. FIG. 7H shows that no GPI anchors were found on multi-transmembrane proteins. Transmembrane domain length is reported as the average of all transmembrane domains for a single protein. Proteins were separated by the membrane which they localize to: the plasma membrane (PM) or internal membranes (IM). Error bars represent the SEM.

FIG. 8A-8G shows that, for peripheral membrane proteins, number of posttranslational modifications can vary between proteins found in lipid rafts (Raft, from Raftprot 2.0), EVs (Exo, from Exocarta), and all human membrane proteins (All, from Swiss-Prot). Specifically, the average number of disulfides (FIG. 8A), phosphoryl groups (FIG. 8B), sugars (glycosylation) (FIG. 8C), palmitoyls (FIG. 8D), myristoyls (FIG. 8E), prenyls (FIG. 8F), and GPI anchors (FIG. 8G) on each protein were calculated. Proteins were separated by the membrane which they localize to: the plasma membrane (PM) or internal membranes (IM). Error bars represent the SEM.

FIG. 9A-9G shows that transmembrane domain lipidation, length, and surface area of LAT affect cellular trafficking, lipid raft association, and extracellular vesicle loading. FIG. 9A provides a schematic of workflow to assess cellular trafficking, lipid-raft association, and protein loading into EVs for the model transmembrane protein, LAT. FIG. 9B shows a cartoon depicting how confocal images of transfected HEK293FT cells were analyzed. Cells were stained with a plasma membrane dye (Cell Mask) and HaloTag-ligand, protein dye (TMR), imaged, and analyzed pixel-by-pixel to determine colocalization of dyes. FIG. 9C shows that using the approach in FIG. 9B, Pearson's coefficient (R) capturing covariance between the plasma membrane and the HaloTag signal are plotted for each construct. Data are reported in box and whisker plots collected from 30 cells from two independent experiments; each symbol is a single cell. A one-way ANOVA was performed to compare plasma membrane localization for each protein, and comparisons were evaluated using the Sidak multiple comparisons correction (*, p<0.05). Comparisons not shown were not significant. FIG. 9D provides a cartoon depicting how confocal images of HaloTag-labeled protein in GPMVs were analyzed. Protein was visualized with a HaloTag ligand-conjugated dye (TMR) and the lipid disordered region of the GPMV was labeled with DiO. FIG. 9E shows protein partitioning into lipid rafts, here reported as L_{O} (liquid-ordered domain) enrichment, for LAT constructs. LAT-dCore was not observed to associate with GPMV membranes, likely due to poor localization to the plasma membrane. Data reported are the average and standard deviation of at least 15 GPMVs from three independent experiments. A one-way ANOVA was performed to compare raft localization for each protein, and comparisons were evaluated using the Sidak multiple comparisons correction (****, p<0.0001). Comparisons not shown were not significant. FIG. 9F shows loading of LAT proteins into EV subpopulations as assessed via SDS-PAGE of fluorescently labelled HaloTag constructs (FIG. 13). Each lane of the protein gel received equal numbers of vesicles. n=2, error bars represent the SEM. FIG. 9G shows EV partitioning of LAT proteins was calculated by normalizing EV protein loading by expression of each construct in EV producer cells as determined by SDS-PAGE (FIG. 13). Values were normalized by WT LAT MV partitioning. n=2, error bars represent the SEM.

FIG. 10A-10B shows how live-cell protein-localization analysis pipeline used in this study enables quantification of protein colocalization with the plasma membrane or endoplasmic reticulum. FIG. 10A, 10B shows cells transfected with (FIG. 10A) LAT-WT and (FIG. 10B) C26A LAT are pictured as an example. HEK293FT cells were transfected with each construct and labeled with (III) ER Tracker Blue-White DPX and (IV) Cell Mask Plasma Membrane Dye. Cells were then imaged (I) and colocalization of protein (II) with each dye (endoplasmic reticulum (III) and plasma membrane (IV)) was determined using Nikon Elements Analysis software.

FIG. 11A-11C shows how Pearson's coefficients (R) between labeled transgenic protein and the plasma membrane and endoplasmic reticulum enables quantification of protein trafficking. The plasma membrane was stained with Cell Mask Plasma Membrane Dye, and the endoplasmic reticulum was labeled with ER Tracker Blue-White DPX dyes. The transfected protein construct was either labeled with TMR (FIG. 11A, FIG. 11C) or directly visualized by mRFP1 fluorescence (FIG. 11B). The data in panel a, b, and c correspond to experiments from FIG. 9, FIG. 14, and FIG. 16, respectively. Data are reported in box and whisker plots collected from 30 cells from two independent experiments; each symbol is a single cell.

FIG. 12 shows how giant plasma membrane vesicle (GPMV) analysis pipeline enables an evaluation of protein association with lipid rafts. HEK293FT cells were transfected with each construct, treated with vesiculation agents, and stained with DiO (non-raft stain). Protein association with lipid rafts was determined by measuring the protein's fluorescence intensity in the raft region (low DiO fluorescence) and nonraft region (high DiO fluorescence) using line scan analysis and using these values to calculate L_{O} enrichment. L_{O} enrichment values above 0 indicate proteins prefer lipid rafts.

FIG. 13 shows uncropped protein gels and standard curves generated from LAT transmembrane protein gels that show construct expression and loading for cell lysates and EVs, respectively. Analyzed data is presented in FIG. 9. Values below the standard indicate the number of recombinant proteins added to the each well divided by 5×10⁸ (the number of vesicles added to the other wells).

FIG. 14A-14E shows that transmembrane domain length of de novo designed proteins affects cellular trafficking and extracellular vesicle loading. FIG. 14A shows the structure of synthetic, de novo designed transmembrane domain proteins assessed. Two families of proteins were assessed, either containing four or twelve transmembrane domains. Different lengths of transmembrane domains in each family were assessed. FIG. 14B shows Pearson's coefficient (R) from confocal microscopy images evaluating the subcellular localization of mRFP1-tagged de novo designed proteins with a plasma membrane-staining dye, Cell Mask. Data are reported in box and whisker plots collected from 30 cells from two independent experiments; each symbol is a single cell. A one-way ANOVA was performed to compare plasma membrane localization within each protein family, and comparisons were evaluated using the Sidak multiple comparisons correction (dimeric 4TMD and hexameric 12TMD TMD designs) (*, p<0.05; ****, p<0.0001). FIG. 14C shows partitioning of de novo designed proteins with lipid-ordered lipid raft regions of GPMVs as determined by confocal microscopy analysis. Raft association did not significantly differ among de novo designed proteins (one-way ANOVA, Sidak multiple comparisons correction). Both 24 Å constructs did not associate with GPMV membranes. Data reported are the average and standard deviation of at least 20 GPMVs from three independent experiments. FIG. 14D, 14E shows protein loading and partitioning into EVs as calculated by western blots probing the 3× FLAG tag (FIG. 15A-15F). Each lane of the protein gel received equal numbers of vesicles. FIG. 14E shows EV partitioning was calculated by normalizing EV protein loading by expression of each construct in EV producer cells as determined by western blot. EV partitioning values were normalized by 24 Å 4TMD MV partitioning. n=2, error bars represent the SEM.

FIG. 15A-15F shows uncropped western blots and standard curves generated from de novo designed transmembrane protein gels which show construct expression and loading for cell lysates and EVs, respectively. FIG. 15A shows uncropped western blot of cell lysates. FIG. 15B shows a protein standard with a 3×-FLAG tag and HaloTag was run against purified HaloTag (Promega) on an SDS-PAGE gel to quantify the concentration (and equivalent proteins/vesicle) of the protein standard. FIG. 15C, 15D Proteins loaded into vesicles and protein standards were visualized via western blot. Constructs labeled with 3× FLAG tag. FIG. 15E shows protein standard and FIG. 15F shows standard curves used to quantify vesicle loading. Analyzed data are presented in FIG. 14D-14E.

FIG. 16A-16H shows that altering peripheral membrane protein lipidation state affects cellular trafficking, lipid raft association, and extracellular vesicle loading. FIG. 16A shows the effect of four lipidation tags on HaloTag membrane interactions and EV loading was assessed. As determined by confocal microscopy, lipidation alters protein association with the plasma membrane (FIG. 16B) and lipid rafts (FIG. 16C). FIG. 16B shows Pearson's coefficient (R) capturing the covariance between the plasma membrane and the HaloTag signal are plotted for each construct. Data are reported in box and whisker plots collected from 30 cells from two independent experiments; each symbol is a single cell. A one-way ANOVA was performed to compare plasma membrane localization between each lipidated constructs and soluble HaloTag, and comparisons were evaluated using the Sidak multiple comparisons correction (*, p<0.05; ***, p<0.001, ****, p<0.0001). FIG. 16C reported L_{O} enrichment is the average and standard deviation of at least 15 GPMVs from three independent experiments. A one-way ANOVA was performed to compare raft localization for each protein, and comparisons were evaluated using the Sidak multiple comparisons correction (*, p<0.05; **, p<0.01). Comparisons not shown were not significant. FIG. 16D, 16E show protein loading and partitioning of lipidated HaloTag constructs into EVs as calculated by SDS-PAGE (FIG. 17). Each lane of the protein gel received equal numbers of vesicles. FIG. 16E shows EV partitioning was defined by normalizing EV protein loading by expression of each construct in EV producer cells as determined by SDS-PAGE. EV partitioning values were normalized by Sol MV partitioning. Lipidation state of HaloTag affects the number of microvesicles (FIG. 16F) generated but not exosomes (FIG. 16G), as assessed by NTA (Table 9). For 16D-16G, n=2; error bars represent the SEM. FIG. 16H shows the number of microvesicles produced is strongly correlated with EV partitioning of HaloTag constructs.

FIG. 17 shows uncropped protein gels and standard curves generated from peripheral membrane proteins show construct expression and loading profiles for cell lysate and EVs, respectively. Analyzed data are presented in FIG. 16A-16H.

FIG. 18A-18C demonstrates plasma membrane localization and lipid raft association are strong indicators for protein partitioning into EVs. FIG. 18A shows Pearson's coefficients for protein loading and partitioning into MVs and exosomes for all variables measured. Protein loading (proteins per vesicle) and partitioning (protein loading normalized by total protein expression in EV producer cells) for LAT proteins (LAT WT, LAT C26A, LAT dCore, LAT High ASA), de novo designed proteins (24 Å 4TMD, 32 Å 4TMD, 40 Å 4TMD, 24 Å 12TMD, 40 Å 12TMD), and lipid tagged HaloTag proteins (Sol, M, PM, G, PPF) were compared to protein expression in lysate, localization to the plasma membrane and ER, association with lipid rafts, and vesicle number produced and size. FIG. 18B, 18C show plasma membrane localization, EV partitioning, raft localization, and protein loading were also plotted in multivariable plots for (FIG. 18B) MVs and (FIG. 18C) exosomes. Plasma membrane localization is calculated as the Pearson's coefficient between plasma membrane stain and each protein; EV partitioning was calculated by normalizing proteins loaded into EVs by expression of each construct in EV producer cells as determined by SDS-PAGE (FIG. 9G, 14E, 16E). Proteins which associated more strongly with lipid rafts are represented in darker blue. Grey points represent constructs for which a raft localization value could not be determined. The size of each point corresponds to the number of proteins per vesicle as calculated by SDS PAGE (FIG. 9F, 14D, 15D).

FIG. 19A-19C shows that correlation of protein loading and partitioning into MVs and Exos reveal EV-loading design principles. All panels quantify correlation using a Pearson's coefficient. FIG. 19A shows protein loading and partitioning for LAT proteins (LAT WT, LAT C26A, LAT dCore, LAT High ASA). FIG. 19B shows de novo designed proteins (24 Å TMD4, 32 Å TMD4, 40 Å TMD4, 24 Å TMD12, 40 Å TMD12). FIG. 19C shows lipid tagged HaloTag proteins (Sol, M, PM, G, PPF). For all cases, protein loading and portioning were correlated with protein expression in lysate, localization to the plasma membrane and endoplasmic reticulum, association with lipid rafts, and vesicle number and size.

FIG. 20A-20F demonstrates that engineered lipidation of transcription factors enables loading into EVs and functional delivery to recipient cells. FIG. 20A shows a schematic of workflow for evaluating the loading into EVs and functional activity of lipidated synthetic transcriptional factors (SynTFs). Reporter cells express a red fluorescent protein, dsRedExpress2 (dsRedExp2), when activated. FIG. 20B shows activity of SynTF variants when plasmids were directly transfected into reporter cells. Left, reporter expression measured by flow cytometry in absolute units of mean molecules equivalent of phycoerythrin-texas red (PE-TR) (MEPTRS). Each symbol is a biological replicate and error bars are the SEM. A one-way ANOVA was performed, and comparisons were evaluated using the Sidak multiple comparisons correction (**** p<0.0001). SynTFs were compared to the Sol condition; comparisons not shown were not significant. Right, scatter plot for the experiment on the left plotted against band intensity of SynTF expression in those cells as measured by western blot (FIG. 22). FIG. 20C shows estimated number of SynTFs per EV as determined by semi-quantitative western blots using a 1× FLAG tag (FIG. 22). Band intensities from the western blot were divided by the number of EVs added to the western blot as determined by NTA. A recombinant 1× FLAG-tagged protein was used as the protein standard. n=2 FIG. 20D shows representative micrograph of reporter cells activated via EV-mediated delivery of lipidated SynTFs. Scale bar is 100 μm. FIG. 20E shows flow cytometry dot plots for a representative sample of reporter cells treated with either EVs isolated from Mock cells (transfected with filler plasmid; no SynTF) or from cells expressing PM-tagged SynTF. Annotations state the percentage of reporter cells that expressed dsRedExp2. FIG. 20F shows activation of reporter cells treated with SynTF-containing EVs (evaluated by flow cytometry). Each symbol represents a biological replicate, and error bars represent SEM. A one-way ANOVA was performed, and comparisons were evaluated using the Sidak multiple comparisons correction (**** p<0.0001). SynTFs were compared to the Mock condition; comparisons not shown were not significant. Data in panel 20C, 20E, and 20F are representative of two independent experiments. Data in panel 20B were collected from one experiment.

FIG. 21A-21C shows how flow cytometry gating and workflow enable quantification of relevant data. FIG. 21A shows that events collected during a typical flow cytometry experiment were first gated based on side scatter area (SSC-A) and forward scatter area (FSC-A) to identify HEK293FT cells. Single cells were then identified by forward scatter height (FSC-H) vs FSC-A discrimination. Single color compensation controls were not gated further prior to generating a compensation matrix. For SynTF experiments, HEK293FTs expressing no fluorescent proteins were then used to define the miRFP720+ gate (i.e., reporter cells with an active locus), typically such that <0.1% of HEK293FTs were considered miRFP720+. FIG. 21B shows, Left, a sample histogram of single, reporter cells as gated in FIG. 21A. Right, reporter cells that did not receive any SynTF were used to establish a dsRedExpress2+ gate. FIG. 21C shows how to calibrate fluorescent intensity data, UltraRainbow Calibration Particles were run alongside cell samples for each SynTF experiment. Beads were first gated based on SSC-A vs FSC-A (left), and then each bead population was gated using two fluorescent channels, typically PE-Texas Red and Pacific Blue. The mean fluorescence (MF) of each grouping was plotted against the vendor-supplied number of equivalent fluorophores (i.e., mean equivalent phycoerythrin-Texas red, MEPTRS) and a linear regression was performed with the intercept set to 0. A sample regression equation and goodness of fit are provided.

FIG. 22 shows uncropped protein gels and standard curves for EV-mediated SynTF delivery experiments show construct expression and loading profiles for lysates from EV-producer cells and EVs, respectively. Western blots are probing 1× FLAG tag on the constructs. Protein standard is purified p53 with a 1× FLAG tag (R&D Systems). Analyzed data are presented in FIG. 20C.

FIG. 23 shows that specific lipidated SynTF variants induce reporter expression when delivered via EVs. Representative dot plots for each condition from the experiment described in FIG. 20A-20F. The plots depict forward scatter area (FSC-A) vs reporter expression (dsRedExpress2) for cells treated with EVs containing lipidated SynTFs, where each dot represents an individual reporter cell. The lipidated SynTF tag designation is denoted in the upper right-hand corner of each dot plot, and the percent of reporter cells activated is denoted in the lower right hand corner of each dot plot. The Mock and PM conditions are also shown in FIG. 20E.

### DETAILED DESCRIPTION

Engineered vesicles (EngVs), including, for example, exosomes and microvesicles, are small lipid enclosed carriers of biomolecular cargos, such as polypeptides, lipids, and nucleic acids. EngVs can be secreted by cells and facilitate both intracellular communication as well as biomolecular cargo transport. Due to certain EngVs innate properties (e.g., intrinsic tissue-homing capabilities), EngVs have become highly considered for use in the delivery of therapeutic payloads as biomolecular cargo (e.g., polypeptides, therapeutic polypeptides, nucleic acids). However, there are a plurality of challenges in using EngVs as delivery vehicles, such as stably encapsulating or associating therapeutically effective amounts of biomolecular cargos, such as therapeutic polypeptides or nucleic acids, into or with an EngV without compromising the functional activity of the biomolecular cargos (e.g., by disrupting its structure and/or localization in the cell).

The present disclosure provides, among other things, improved methods and compositions of loading an EngV with biomolecular cargos, a loaded EngV or a plurality thereof produced in accordance with the methods described herein, compositions comprising such a loaded EngV or a plurality thereof, and methods of treating a disease and/or disorder by administering a loaded EngV or a plurality thereof or compositions of the present disclosure.

It is to be appreciated that certain aspects, modes, embodiments, variations and features of the present methods are described below in various levels of detail to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular form “a,” “an,” and “the” include singular and plural references unless the context clearly dictates otherwise. For example, the term “an engineered vesicle” includes a single engineered vesicle as well as a plurality of engineered vesicles, including mixtures thereof.

“As used herein, the term “administration” of an agent or composition to a subject includes any route of introducing or delivering the agent or composition to a subject to perform its intended function. Administration can be carried out by any suitable route, including, but not limited to, intravenously, intramuscularly, intraperitoneally, subcutaneously, and other suitable routes as described herein. Administration can include self-administration and administration by another.

As used herein, the term “agent,” refers to an entity (e.g., for example, a lipid, metal, nucleic acid, polypeptide, polysaccharide, small molecule, etc., or complex, combination, mixture or system ([e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof (e.g., an engineered vesicle). Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. For example, an agent may be utilized in isolated or pure form; an agent may be utilized in crude form.

As used herein, the term “approximately” or “about” means plus or minus 10% as well as the specified number. For example, “about 10” should be understood as both “10” and “9-11”.

As used herein, the term an “amino acid substitution” or “substituted” (when such term is referred to a substituted amino acid) refers to the replacement of at least one existing amino acid residue in a predetermined amino acid sequence with a different amino acid residue in a predetermined amino acid sequence with a different amino acid residue. The term “amino acid insertion” refers to the incorporation of at least one additional amino acid into a predetermined amino acid sequence. While the insertion will usually consist of the insertion of one or two amino acid residues, larger “peptide insertions,” can also be made. The replaced or inserted amino acid residue(s) may be naturally occurring or non-naturally occurring (modified). The term “amino acid deletion” refers to the removal of at least one amino acid residue from a predetermined amino acid sequence.

As used herein, a “conservative amino acid substitution” is one in which the amino acid residue is replaced with an amino acid residue having a similar side chain. Families of amino acid residues having similar side chains have been defined in the art, including: (1) hydrophobic side chains: norleucine, Met, Ala, Val, Leu; (2) neutral hydrophilic side chains: Cys, Ser, Thr, Asn, Gln; (3) acidic side chains: Asp, Glu; (4) basic side chains: His, Lys, Arg; (5) side chains that influence chain orientation: Gly, Pro; and (6) aromatic side chains: Trp, Tyr, Phe. For example, a non-conservative amino acid substitution is a substitution of an amino acid residue with a substantially different side chain (i.e., an amino acid residue that is a member of a different family). In some embodiments, a conservative amino acid substitution is made by considering the hydropathic index of the amino acid residue. Each amino acid is assigned a hydropathic index on the basis of its hydrophobicity and charge characteristics. They are: lie (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gin (−3.5); Asp (−3.5); Asn (−3.5); Lys (−3.9); and Arg (−4.5). The importance of the hydropathic amino acid index in conferring interactive function on a polypeptide is understood in the art (see, e.g., Kyte et al (1982) J Mol Biol 157:105-131). In some embodiments, a conservative amino acid substitution is made by replacing one amino acid residue with another amino acid residue having a the same or similar (e.g., within about +2, +1.5, +1, +0.5, −0.5, −1, −1.5, or −2) hydropathic index. In some embodiments, a conservative amino acid substitution is made by considering the hydrophilicity of the amino acid residue. The following hydrophilicity values have been assigned: Arg (+3.0); Lys (+3.0±1); Asp (+3.0±1); Glut (+0.2); Gly (0); Thr (−0.4); Pro (−0.5+1); Ala (−0.5); His (−0.5); Cys (−1.0); Met (−1.3); Val (−1.5); Leu (−1.8); lie (−1.8); Tyr (−2.3); Phe (−2.5); and Trp (−3.4). In some embodiments, a conservative amino acid substitution is made by replacing one amino acid residue with another amino acid residue having a the same or similar (e.g., within about +2, +1.5, +1, +0.5, −0.5, −1, −1.5, or −2) hydrophilicity. Exemplary amino acid substitutions are set forth in

TABLE 1
Conservative amino acid substitutions

| Original | | Preferred |
|---|---|---|
| Residue | Exemplary Substitution | Substitution |
| Ala | Val, Leu, Ile | Val |
| Arg | Lys, Glu, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Nle | Leu |
| Leu | Nle, Ile, Val, Met, Ala, Phe | Ile |
| Lys | Arg, Dbu, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Nle | Leu |

Nle = norleucine
Dbu = 2,4-diaminobutyric acid

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used herein, the term “engineered” refers to the aspect of having been manipulated by the hand of man. For example, a nucleic acid is considered to be “engineered” when two or more sequences that are not linked together in that order in nature are manipulated by the hand of man to be directly linked to one another in the engineered nucleic acid and/or when a particular nucleotide residue in a nucleic acid is non-naturally occurring and/or is caused through action of the hand of man to be linked with an entity or moiety with which it is not linked in nature (e.g., a nucleic acid encoding a fusion polypeptide described herein). Comparably, a vesicle can be considered to be “engineered” (e.g., an “EngV”) if it has been subjected to a manipulation, so that its genetic, epigenetic, and/or phenotypic identity is altered relative to an appropriate reference vesicle such as an otherwise identical vesicle that has not been so manipulated. In some embodiments, the manipulation is or comprises a genetic manipulation, so that its genetic information is altered (e.g., new genetic material not previously present has been introduced, for example by transformation, transfection, transduction, or other mechanism, or previously present genetic material is altered or removed, for example by substitution or deletion mutation) or by chemical or physical manipulation (e.g., exposure to a specific biomolecule, such as lipids in growth medium). In some embodiments, an engineered vesicle is one that has been manipulated so that it contains and/or expresses a particular agent of interest (e.g., a biomolecular cargo, a protein, a nucleic acid, and/or a particular form thereof) in an altered amount and/or according to altered timing relative to such an appropriate reference vesicle. As is common practice and is understood by those in the art, progeny of, for example, an engineered polynucleotide or cell are typically still referred to as “engineered” even though the actual manipulation was performed on a prior entity.

As used herein, the term “expression” of a nucleic acid sequence refers to the generation of any gene product from the nucleic acid sequence. A gene product can be a transcript. A gene product can be a polypeptide. Expression of a nucleic acid sequence can involve one or more of the following: (1) production of an RNA template from a DNA sequence (e.g., by transcription); (2) processing of an RNA transcript (e.g., by splicing, editing, etc.); (3) translation of an RNA into a polypeptide; and/or (4) post-translational modification of a polypeptide.

As used herein, the term “fragment” refers to a material or entity as described herein that has a structure that includes a discrete portion of the whole, but lacks one or more moieties found in the whole. For example, a fragment can consist of such a discrete portion. A fragment can consist of or comprise a characteristic structural element or moiety found in the whole. For example, a polypeptide fragment can comprise or consist of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polypeptide. A polypeptide fragment can comprise or consist of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polypeptide. The whole material or entity may be referred to as the “parent” of the fragment.

As used herein, the term “functional”, refers to a form or fragment of an entity (e.g., biomolecular cargo) that exhibits a particular property and/or activity.

As used herein, the term “effective amount” or “therapeutically effective amount” refers to a quantity of an agent or composition sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the engineered vesicles administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

As used herein, the terms “improved”, “increased”, or “reduced”, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, the term “isolated”, “purified”, or “biologically pure” refers to material that is free to varying degrees from components which normally accompany it as found in its native and/or original state (e.g., undesired sample components). “Isolate” denotes a degree of separation from original source or surroundings. “Purify” denotes a degree of separation that is higher than isolation. For example, a “purified” or “biologically pure” polypeptide is sufficiently free of other materials such that any impurities do not materially affect the biological properties of the polypeptide or cause other adverse consequences. That is, an EngV (e.g., a loaded EngV) or a plurality thereof of the presently disclosed subject matter is purified if it is substantially free of undesired sample components, such as cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis. The term “purified” can denote that a nucleic acid or polypeptide gives rise to essentially one band in an electrophoretic gel. For a polypeptide that can be subjected to modifications, for example, phosphorylation or lipidation, different modifications may give rise to different isolated polypeptides, which can be separately purified.

As used herein, the term “loading” or “loaded” refers to the introduction of a biomolecular cargo and/or a fusion polypeptide (e.g., as described herein) into, onto, or otherwise associated with an EngV, such that the biomolecular cargo and/or fusion polypeptide is encapsulated in or associated with the EngV. As described herein, in some aspects, an EngV loaded with a biomolecular cargo and/or fusion polypeptide refers to an EngV where the biomolecular cargo and/or fusion polypeptide is linked, non-covalently associated, or conjugated to the exterior surface of the EngV.

As used herein, the term “operably linked”, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

As used herein, the term “percent (%) amino acid sequence identity” or “percent sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity. Alignment for purposes of determining percent amino acid sequence identity can be achieved in various ways that are known in the art, for instance, using publicly available computer software such as BFASTp, BFAST-2, AFIGN (e.g., AFIGN-2) or Megalign (DNASTAR) software. To obtain gapped alignments for comparison purposes, Gapped BEAST can be utilized as described in Altschul et al, (1997) Nucleic Acids Res. 25 (17): 3389-3402. In addition, the percent identity between two amino acid sequences can be determined using the Needleman and Wunsch (J. Mol. Biol. (48): 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for a particular route of administration.

The terms “polypeptide”, “peptide”, and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full-length proteins or fragments thereof, wherein the amino acid residues are linked by covalent peptide bonds.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

As used herein, a “reference” entity, system, amount, set of conditions, etc., is one against which a test entity, system, amount, set of conditions, etc. is compared as described herein. For example, in some embodiments, a “reference” polypeptide is a control polypeptide, e.g., a biomolecular cargo that does not comprise a lipid tag, a biomolecular cargo that is not transmembrane modified. In some embodiments, a reference or control is tested and/or determined simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the terms “subject”, “individual”, or “patient” are used interchangeably and refer to an individual organism, a vertebrate, or a mammal and may include humans, non-human primates, rodents, and the like (e.g., which is to be the recipient of a particular treatment). In certain embodiments, the individual, patient or subject is a human.

As used herein, the term “vector” has the same meaning as commonly understood by one of ordinary skill in the art, and refers to a nucleic acid molecule capable of transporting another nucleic acid molecule to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors used in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### Methods of Loading Engineered Vesicles (EngVs)

Engineered vesicles (EngVs) include a heterogeneous population of lipid-bound nanoparticles released by nearly all cell types. EngVs include, for example, extracellular vesicles (EVs) such as microvesicles (MVs), which are released through budding of the plasma membrane, and exosomes (Exos) derived from the endosomal compartment. EngVs can also include, for example, enveloped virus like particles and enveloped gene delivery vectors, such as lentivirus, which also bud from the plasma membrane in a manner that mimics that of microvesicle budding. EngVs have become highly considered for use in the delivery of therapeutic payloads as biomolecular cargo (e.g., polypeptides, nucleic acids, viral particles or cores). However, there are a plurality of challenges in using EngVs as delivery vehicles, such as stably loading biomolecular cargo (e.g., in therapeutically effective amounts) without compromising the functional activity of the biomolecular cargo (e.g., by disrupting its structure and/or localization in the cell).

The present disclosure provides, among other things, improved methods of loading an EngV with a biomolecular cargo or a fusion polypeptide comprising (i) a biomolecular cargo, and (ii) one or more lipid tags. Methods of the present disclosure can efficiently load an EngV or a plurality thereof with a therapeutically effective amount of a biomolecular cargo (e.g., of a fusion polypeptide described herein) and/or without comprising its functional activity.

In one aspect, such methods of loading (making) an engineered vesicle (EngV) with a fusion polypeptide can comprise contacting a cell with a nucleic acid encoding a fusion polypeptide comprising (i) a biomolecular cargo, and (ii) one or more lipid tags, wherein contacting occurs under conditions sufficient to permit (i) uptake of the nucleic acid by the cell, (ii) translation of the fusion polypeptide, and (iii) post-translational modification of the one or more lipid tags with one or more lipids by endogenous host machinery in the cell to thereby generate a lipid-modified fusion polypeptide, and wherein the lipid-modified fusion polypeptide is directed and loaded into the EngV. In some embodiments, the biomolecular cargo comprises a transmembrane domain and the transmembrane domain is modified (e.g., by one or more of: changing the length of the transmembrane domain and changing the transmembrane domain surface area) to enhance direction and loading into or on the EngV relative to the biomolecular cargo with an unmodified transmembrane domain.

In another aspect, such methods of loading (making) an engineered vesicle (EngV) with a biomolecular cargo can comprise contacting a cell with a nucleic acid encoding the biomolecular cargo, wherein contacting occurs under conditions sufficient to permit (i) uptake of the nucleic acid by the cell, and (ii) translation of the biomolecular cargo, wherein the biomolecular cargo comprises a transmembrane domain and the transmembrane domain is modified to improve direction and loading into or on the EngV relative to the biomolecular cargo with an unmodified transmembrane domain, thereby generating a transmembrane modified biomolecular cargo, and wherein the transmembrane modified biomolecular cargo is directed and loaded into or on the EV. In some embodiments, the transmembrane domain is modified by one or more of: changing the length of the transmembrane domain and changing the transmembrane domain surface area. In some embodiments, the transmembrane modified biomolecular cargo further comprises one or more lipid tags that can be post-translationally modified with one or more lipids by endogenous host machinery in the cell to thereby generate a lipid-modified, transmembrane modified biomolecular cargo, and wherein the lipid-modified, transmembrane modified biomolecular cargo is directed and loaded into or on the EV.

Without wishing to be bound by any one theory, technologies of the present disclosure enable the improved loading of a biomolecular cargo (e.g., of a fusion polypeptide described herein) into or with the EngV by engineering and/or directing biomolecular cargo association with lipid rafts. Lipid rafts, or ordered lipid microenvironments, comprise certain membrane components (e.g., cholesterol and sphingolipids) and have been shown to play important roles in polypeptide organization and recycling as well as in EngV biogenesis. Accordingly, compositions and methods of the present disclosure comprise engineering biomolecular cargo and/or fusion polypeptides comprising biomolecular cargo. Such biomolecular cargo and/or fusion polypeptides can comprise one or more lipid tags that become covalently modified with lipids by endogenous host cell machinery after translation, thereby producing a lipid-modified biomolecular cargo or fusion polypeptide. Such lipid-modified biomolecular cargo or fusion polypeptide can associate with lipid rafts, which is understood to enhance biomolecular cargo and/or fusion polypeptide partitioning to lipid rafts and subsequent loading into or on EngVs. Surprisingly, such loaded biomolecular cargo (e.g., of a fusion polypeptide described herein) can maintain its functional activity (e.g., compared to an appropriate reference, such as a biomolecular cargo lacking a lipid tag) and/or be loaded with therapeutically effective amounts of the biomolecular cargo.

Alternatively or additionally, compositions and methods of the present disclosure can comprise engineering biomolecular cargo (e.g., of a fusion polypeptide described herein) by modifying the biomolecular cargo transmembrane domain (e.g., changing the length of the transmembrane domain, changing the transmembrane domain surface area), thereby producing a transmembrane modified biomolecular cargo. Such transmembrane modified biomolecular cargo (e.g., of a fusion polypeptide described herein) can associate with lipid rafts, which is understood to enhance biomolecular cargo (e.g., of a fusion polypeptide described herein) partitioning into lipid rafts and subsequent loading into or on EngVs. Also unexpectedly, such loaded transmembrane modified biomolecular cargo (e.g., of the fusion polypeptide described herein) can maintain its functional activity (e.g., compared to an appropriate reference, such as a biomolecular cargo with an unmodified transmembrane domain) and/or be loaded with therapeutically effective amounts of the biomolecular cargo.

Changing the length of the transmembrane domain can include, for example, lengthening or shortening the transmembrane domain which can be accomplished by inserting or deleting amino acids. In some embodiments, about 10 amino acids, about 15 amino acids, about 20 amino acids, about 30 amino acids, about 35 amino acids, about 40 amino acids, about 45 amino acids, or about 50 amino acids are inserted or deleted from the transmembrane domain. In some embodiments, the transmembrane domain is lengthened by insertions of hydrophobic amino acids. Without wishing to be bound by any one theory, it is understood that hydrophobic amino acids prefer the lipid membrane environment.

Changing the transmembrane domain surface area can include, for example, changing the transmembrane domain length (e.g., as described herein) and/or substituting one or more amino acid residues of the transmembrane domain with another (e.g., the comparable hydrophobicity) that has a larger or smaller side chain. Substitution to an amino acid with a larger side chain would increase the transmembrane domain surface area, while substitution to an amino acid with a smaller side chain would decrease the transmembrane domain surface area. Alternatively or additionally, the transmembrane domain surface area can be changed by inserting additional or deleting transmembrane domains (e.g., by modifying the transmembrane amino acid sequence or engineering polypeptides which oligomerize to form different sized complexes).

In some embodiments, methods of the present disclosure further comprise isolating an EngV (e.g., a loaded EngV) or a plurality thereof from the cells and/or other undesirable sample components, such as cell culture media and/or cellular debris. A plurality of EngV isolation methods are known in the art, including those that can specifically isolate exosomes, microvesicles, enveloped virus like particles, or enveloped gene delivery vectors (e.g., lentivirus). One of ordinary skill, reading the present disclosure, would readily recognize and understand how to select and use such methods in accordance with technologies of the present disclosure. Examples of methods for isolating an EngV (e.g., a loaded EngV) or a plurality thereof include, without limitation, size exclusion chromatography, filtration (e.g., tangential flow filtration), asymmetric flow field-flow fractionation, ion-exchange chromatography, affinity chromatography, ultracentrifugation (e.g., density gradient ultracentrifugation), and/or precipitation.

In some embodiments, isolating an EngV or a plurality thereof comprises separation of an EngV or plurality thereof from one or more undesired sample components by chromatographic methods (e.g., size exclusion chromatography (SEC), ion-exchange chromatography, affinity chromatography). SEC is a well-established technique used for separation of sample components based on molecular size or hydrodynamic volumes. SEC can be used for isolating an EngV or a plurality thereof from a large variety of sample types, including, for example, blood-based samples, ascites, and cell culture-derived samples. Typically, the stationary phase includes cross-linked agarose beads (e.g., Sepharose®). See, e.g., Liangsupree T. et al., J Chromatogr A. 2021 Jan. 11; 1636:461773). Ion-exchange chromatography is commonly used to separate charged biological molecules. EVs can be negatively charged on their surface and bound to a positively charged chromatographic matrix and then eluted. See, e.g., Heath N. et al., Sci Rep. 2018 Apr. 10; 8 (1): 5730). Affinity chromatography is separation method based on a specific binding interaction between an immobilized ligand and its binding partner. See, e.g., Balaj L. et al., Sci Rep. 2015 May 19; 5:10266).

In some embodiments, isolating an EngV or a plurality thereof comprises separation of an EngV or a plurality thereof from one or more undesired sample components by filtration (e.g., tangential flow filtration (TFF)). For example, in TFF, a stream of fluids containing an EngV or a plurality thereof flow tangentially by moving across an ultrafiltration membrane, but not directly through the membrane. Molecules smaller than the molecular weight cut-off travel through the membrane and are discarded, while molecules larger than the cut-off level, such as an EV, remain on the membrane and are recirculated and concentrated. See, e.g., Liangsupree T. et al., J Chromatogr A. 2021 Jan. 11; 1636:461773).

In some embodiments, isolating an EngV or a plurality thereof comprises separation of an EngV or a plurality thereof from one or more undesired sample components by asymmetric flow field-flow fractionation (AF4). AF4 fractionates extracellular particles (e.g., EngVs) based on hydrodynamic sizes and demonstrates a capability to separate particles with sizes ranging from a few nanometers to undefined level of micrometers. See, e.g., Zhang H. et al., Nat Protoc. 2019 April; 14 (4): 1027-1053).

In some embodiments, isolating an EngV or a plurality thereof comprises separation of an EngV or a plurality thereof from one or more undesired sample components by ultracentrifugation. Ultracentrifugation is based on separation of particles according to their buoyant density. To isolate an EngV or a plurality thereof, several ultracentrifugation steps are typically conducted. First, the particles with high buoyant density, cellular debris, aggregates of biopolymers, and other structures with densities higher than an EngV can be sedimented. The resulting supernatant can then be ultracentrifuged to yield an EngV pellet. Density gradient ultracentrifugation can be utilized, wherein a continuous density gradient (e.g., a sucrose or iodixanol density gradient) and differential centrifugation can be conducted. In some embodiments, an EngV or a plurality thereof can be further purified using filtration and/or subsequent washing steps. See, e.g., Zhao Z. et al., Extracell Vesicles Circ Nucl Acids. 2021; 2:80-103).

In some embodiments, isolating an EngV or a plurality thereof comprises separation of an EngV or a plurality thereof from one or more undesired sample components by precipitation. For example, use of hydrophilic polymers, such as polyethylene glycol (PEG), can reduce solubility by lowering the hydration of an EngV or a plurality thereof, leading to precipitation. See, e.g., Zhao Z. et al., Extracell Vesicles Circ Nucl Acids. 2021; 2:80-103).

An EngV (e.g., a loaded EngV) or a plurality thereof can be isolated to a certain degree of purity. An EngV or plurality thereof produced in accordance with methods described herein can be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other undesirable sample components (e.g., cellular components, cellular debris). In some embodiments, an EngV or a plurality thereof produced in accordance with methods described herein is about 80%, about 85%, about 90%, about 91%, about 92%, about 93%, about 94%, about 95%, about 96%, about 97%, about 98%, about 99%, or more than about 99% pure.

Conditions sufficient to permit (i) uptake of the nucleic acid by the cell, (ii) translation of the biomolecular cargo or fusion polypeptide, and optionally, (iii) post-translational modification of the one or more lipid tags with one or more lipids by endogenous host machinery in the cell to thereby generate a lipid-modified fusion polypeptide are well known in the art and the skilled artisan would recognize a variety of techniques and/or cell culture conditions that could be successfully utilized. Such techniques include, for example, and without limitation, microinjection, bolistic methods, laserfection/optical transfection, transduction (e.g., using viral vectors), and transfection. The choice of such techniques and/or cell culture conditions can depend on a plurality of factors, such as the type and origin of cells and the form of contacted nucleic acid. Selection of such techniques and/or cell culture conditions are well within the level of the skilled artisan. See, e.g., Fus-Kujawa A. et al., Front Bioeng Biotechnol. 2021 Jul. 20; 9:701031

### Biomolecular Cargos

As will be apparent to those of ordinary skill in the art, biomolecular cargos (and nucleic acids encoding biomolecular cargos) of the present disclosure can comprise (or encode) any suitable biomolecular cargo known in the art. Non-limiting examples of biomolecular cargos, including biomolecular cargos that can be included in fusion polypeptides of the present disclosure, include therapeutic polypeptides and/or polypeptides that bind therapeutic molecules (e.g., nucleic acids, chemotherapeutic agents). Therapeutic polypeptides can include, for example and without limitation, enzymes (e.g., nucleases), monoclonal antibodies, transcription factors, antigens (e.g., for use in vaccines), fusion proteins, gene therapy vectors (e.g., adeno-associated virus (AAV)) and/or cytokines. In some embodiments, a biomolecular cargo is or comprises a fragment (e.g., a functional fragment) of a therapeutic polypeptide. In some embodiments, a biomolecular cargo is or comprises a zinc finger nuclease or zinc finger transcription factor (e.g., ZF-p65). In some embodiments, a biomolecular cargo is or comprises a fusion protein (e.g., Vesicular stomatitis virus G protein). Non-limiting examples of biomolecular cargo are shown in Table 2.

Biomolecular cargos for use in accordance with technologies described herein (e.g., including biomolecular cargos of fusion polypeptides) can comprise a transmembrane domain and the transmembrane domain can be modified (changed) to improve direction and loading into or on the EngV relative to the biomolecular cargo with an unmodified transmembrane domain, thereby generating a transmembrane modified biomolecular cargo. In some embodiments, the transmembrane domain is modified by changing the length of the transmembrane domain (e.g., shortening or lengthening transmembrane domain length). In some embodiments, the transmembrane domain is modified by changing the transmembrane domain surface area (e.g., increasing or decreasing transmembrane domain surface area). In some embodiments, the transmembrane domain is modified by both of changing the length of the transmembrane domain and changing the transmembrane domain surface area.

In some embodiments, a transmembrane domain modified biomolecular cargo further comprises one or more lipid tags as described herein.

TABLE 2
Examples of Amino Acid Sequences of Biomolecular
Cargo

| Gene Name | Amino Acid Sequence |
|---|---|
| HaloTag | EIGTGFPFDPHYVEVLGERMHYVDVGPR |
| | DGTPVLFLHGNPTSSYVWRNIIPHVAPT |
| | HRCIAPDLIGMGKSDKPDLGYFFDDHVR |
| | FMDAFIEALGLEEVVLVIHDWGSALGFH |
| | WAKRNPERVKGIAFMEFIRPIPTWDEWP |
| | EFARETFQAFRTTDVGRKLIIDQNVFIEG |
| | TLPMGVVRPLTEVEMDHYREPFLNPVD |
| | REPLWRFPNELPIAGEPANIVALVEEYM |
| | DWLHQSPVPKLLFWGTPGVLIPPAEAAR |
| | LAKSLPNCKAVDIGPGLNLLQEDNPDLI |
| | GSEIARWLSTLEISG |
| mRFP1 | MASSEDVIKEFMRFKVRMEGSVNGHEF |
| | EIEGEGEGRPYEGTQTAKLKVTKGGPLP |
| | FAWDILSPQFQYGSKAYVKHPADIPDYL |
| | KLSFPEGFKWERVMNFEDGGVVTVTQD |
| | SSLQDGEFIYKVKLRGTNFPSDGPVMQK |
| | KTMGWEASTERMYPEDGALKGEIKMRL |
| | KLKDGGHYDAEVKTTYMAKKPVQLPG |
| | AYKTDIKLDITSHNEDYTIVEQYERAEG |
| | RHSTGA |
| Engineered | PKKKRKVSGSQYLPDTDDRHRIEEKRKR |
| Transcription | TYETFKSIMKKSPFSGPTDPRPPPRRIAVP |
| Factor | SRSSASVPKPAPQPYPFTSSLSTINYDEFP |
| | TMVFPSGQISQASALAPAPPQVLPQAPAP |
| | APAPAMVSALAQAPAPVPVLAPGPPQA |
| | VAPPAPKPTQAGEGTLSEALLQLQFDDE |
| | DLGALLGNSTDPAVFTDLASVDNSEFQQ |
| | LLNQGIPVAPHTTEPMLMEYPEAITRLVT |
| | GAQRPPDPAPAPLGAPGLPNGLLSGDED |
| | FSSIADMDFSALLGSGGGGSGGGGSGGG |
| | GSGTARPGERPFQCRICMRNFSKGERLV |
| | RHTRTHTGEKPFQCRICMRNFSRMDNLS |
| | THLRTHTGEKPFQCRICMRNFSRKDALN |
| | RHLKTHLRGSGS |

### Fusion Polypeptides

Fusion polypeptides of the present disclosure can comprise (i) a biomolecular cargo (e.g., as described elsewhere herein) and (ii) one or more lipid tags. Fusion polypeptides can comprise one, two, three, four, five, or six lipid tags. The biomolecular cargo can be N-terminal to the one or more lipid tags, C-terminal to the one or more lipid tags, or the one or more lipid tags may be located within the biomolecular cargo. In some embodiments, the one or more lipid tags are C-terminal to the biomolecular cargo. In some embodiments, the one or more lipid tags are N-terminal to the biomolecular cargo. In some embodiments, the one or more lipid tags are located within the biomolecular cargo. In some embodiments, one or more lipid tags are N-terminal to the biomolecular cargo and an additional one or more lipid tags are C-terminal to the biomolecular cargo. In some embodiments, one or more lipid tags are N-terminal to the biomolecular cargo and an additional one or more lipid tags are within the biomolecular cargo. In some embodiments, one or more lipid tags are C-terminal to the biomolecular cargo and an additional one or more lipid tags are within the biomolecular cargo.

In some embodiments, a fusion polypeptide comprises two or more lipid tags that are the same. In some embodiments, a fusion polypeptide comprises two or more lipid tags that are different from one another. In some embodiments, a fusion polypeptide comprises two or more lipid tags that are the same and one or more lipid tags that are different from the two or more lipid tags that are the same.

In some embodiments, a biomolecular cargo comprises two or more polypeptides fused into a single biomolecular cargo (e.g., a transcription factor and a zinc finger may be fused to form a single biomolecular cargo).

### Lipid Tags

Lipid tags of the present disclosure are or comprise short amino acid sequences that can become covalently modified with lipids (e.g., are post-translationally lipidated) by endogenous host cell machinery after the fusion polypeptide is transcribed and translated, or translated (e.g., depending on the nucleic acid encoding the fusion polypeptide utilized). In some embodiments, lipid tags comprise about 10 to about 70 amino acid residues. For example, lipid tags can comprise about 10 to about 70, about 10 to about 60, about 10 to about 50, about 10 to about 40, about 10 to about 30, about 20 to about 70, about 20 to about 60, about 20 to about 50, about 20 to about 40, about 30 to about 70, about 30 to about 60, or about 30 to about 50 amino acid residues.

Lipid tags can be or comprise polypeptides or fragments thereof that endogenously are post-translationally lipidated. Such polypeptides or fragments thereof include, for example, and without limitation, Src kinase, Lyn tyrosine protein kinase, Ras (e.g., K-Ras, N-Ras, H-Ras), paralemmin-1, hemagglutinin, transferrin-1, fyn kinase, Rras2, Rab18, or any combination thereof. Examples of lipid tags are shown in Table 3.

### Post-Translational Modification of Lipid Tags

Post-translational modification of polypeptides (e.g., lipid tags) can include covalent addition of a plurality of lipids (e.g., fatty acids, isoprenoids, cholesterol) to amino acid residues of such polypeptides. Post-translation modification of polypeptides by covalent addition of lipids is also referred to as post-translational lipidation. Post-translational lipidation of polypeptides is important in, for example, the regulation of cellular functions by affecting polypeptide functional activity, localization, and/or stability.

Lipid tags of the present disclosure can be post-translationally modified with one or more lipids by endogenous host cell machinery in the cell to thereby generate a lipid-modified fusion polypeptide. Post-translational modification of the one or more lipid tags with a lipid can comprise one or more of acylation or prenylation.

Polypeptide acylation is the addition of fatty acids to amino acid residues of a polypeptide. There are three major classes of fatty acylation reactions in mammalian cells: myristoylation catalyzed by myristoyl transferase, palmitoylation/acylation catalyzed by DHHC family enzymes, and fatty acylation of secreted proteins catalyzed by MBOAT family enzymes. Each of these fatty acylation reactions utilizes different enzymes, different fatty acyl CoA and polypeptide substrates, and occurs in different intracellular locations.

Post-translational modification by acylation of the one or more lipid tags can comprise myristoylation, palmitoylation, palmitoleoylation, stearoylation, or any combination thereof.

Myristoylation is a lipid modification involving the addition of a 14-carbon saturated fatty acid, myristic acid, to the N-terminal glycine of polypeptides and/or lysine amino acid residues of polypeptides. In some embodiments, the post-translational modification of the one or more lipid tags with a lipid comprises myristoylation.

Palmitoylation is the covalent attachment of fatty acids, such as palmitic acid, to cysteine and in some instances, serine and/or threonine, of polypeptides. In some embodiments, the post-translational modification of the one or more lipid tags with a lipid comprises palmitoylation. In some embodiments, post-translational modification of the one or more lipid tags with a lipid comprises palmitoylation and myristoylation.

Palmitoleoylation is a unique lipid modification of polypeptides, where a monounsaturated fatty acid, palmitoleic acid (C16:1), is covalently attached to polypeptides. In some embodiments, the post-translational modification of the one or more lipid tags with a lipid comprises palmitoleoylation.

Stearoylation is the covalent attachment of a stearoyl group to an amino acid in a polypeptide. In some embodiments, the post-translational modification of the one or more lipid tags with a lipid comprises stearoylation.

Polypeptide prenylation is a ubiquitous, irreversible, and covalent post-translational modification found in all eukaryotic cells, comprising attachment of either a farnesyl or a geranylgeranyl isoprenoid to an amino acid residue. Three prenyltransferase enzymes catalyze this modification. Farnesyltransferase (FTase) and geranylgeranyltransferase type 1 (GGTase-I) catalyze attachment of a single farnesyl (15 carbon) or geranylgeranyl (20 carbon) isoprenoid group to a cysteine residue, while Geranylgeranyltransferase type 2 (GGTase-II or Rab geranylgeranyltransferase) catalyzes the addition of two geranylgeranyl groups to two cysteine residues.

Post-translational modification by prenylation of the one or more lipid tags can comprise farnesylation, geranylgeranylation, or any combination thereof. In some embodiments, the post-translational modification of the one or more lipid tags with a lipid comprises farnesylation. In some embodiments, the post-translational modification of the one or more lipid tags with a lipid comprises geranylgeranylation.

In some embodiments, post-translation modification of the one or more lipid tags with a lipid comprises acylation and prenylation. In some embodiments, post-translational modification of the one or more lipid tags with a lipid comprises myristoylation, palmitoylation, palmitoleoylation, stearoylation, farnesylation, geranylgeranylation, or any combination thereof. In some embodiments, post-translational modification of the one or more lipid tags with a lipid comprises palmitoylation and farnesylation.

### Lipid-Modified Biomolecular Cargos and Fusion Polypeptides

Post-translational modification of the one or more lipid tags with one or more lipids by endogenous host machinery in the cell can thereby generate a lipid-modified fusion polypeptide and/or a lipid modified biomolecular cargo. A lipid-modified fusion polypeptide or biomolecular cargo of the present disclosure can be myristoylated, palmitoylated, palmitoleoylated, stearoylated, geranylgeranylated, farnesylated, or any combination thereof. A lipid-modified fusion polypeptide or biomolecular cargo can include one, two, three, four, five, or six lipid-modifications. In some embodiments, the one or more lipid tags comprise two or more lipid-modifications that are the same. In some embodiments, the one or more lipid tags comprise two or more lipid-modifications that are different from one another. In some embodiments, the one or more lipid tags comprise two or more lipid-modifications that are the same and one or more lipid-modifications that are different from the two or more lipid-modifications that are the same.

In some embodiments, the lipid-modified fusion polypeptide or biomolecular cargo is myristoylated. In some embodiments, the lipid-modified fusion polypeptide or biomolecular cargo is palmitoylated and myristoylated. In some embodiments, the lipid-modified fusion polypeptide or biomolecular cargo is triple palmitoylated. In some embodiments, the lipid-modified fusion polypeptide or biomolecular cargo is geranylgeranylated. In some embodiments, the lipid-modified fusion polypeptide or biomolecular cargo is double palmitoylated and farnesylated.

In some embodiments, the lipid-modified fusion polypeptide comprises a biomolecular cargo comprising a transmembrane domain, wherein the transmembrane domain is modified (changed) to enhance direction and loading into or on the EngV relative to the biomolecular cargo with an unmodified transmembrane domain (e.g., by changing the length of the transmembrane domain and/or changing the transmembrane domain surface area).

### Nucleic Acids

Nucleic acids encoding biomolecular cargo and/or fusion polypeptides of the present disclosure are recombinant polynucleotides (e.g., DNA or RNA). Such recombinant polynucleotides may be prepared by a variety of methods available and known in the art. For example, nucleic acids comprising nucleotide sequences encoding a biomolecular cargo and/or lipid tag of the present disclosure may be excised from DNA using restriction enzymes, may be amplified from plasmids or genomic polynucleotide sequences using, for example, polymerase chain reaction, or may be synthesized (e.g., using chemical synthesis techniques or in vitro transcription). A combination of known methods may also be utilized to prepare a nucleic acid encoding a biomolecular cargo and/or fusion polypeptide of the present disclosure.

Nucleic acids encoding biomolecular cargo and/or fusion polypeptides of the present disclosure can be cloned into a vector capable of expressing a biomolecular cargo and/or fusion polypeptide. A plurality of suitable cloning methods are known in the art, including, for example and without limitation, Gibson assembly and restriction digest and ligation.

A plurality of vectors are also available and known to those of ordinary skill in the art and can be utilized for expression of biomolecular cargo and/or fusion polypeptides provided herein. Vectors for use in accordance with the present disclosure can be non-viral or viral vectors. The choice of expression vector can be influenced by the choice of expression system (e.g., in vitro, in vivo, cell-type). Such selection is well within the level of skill of one of ordinary skill in the art. In general, vectors can include transcriptional promoters and optionally, for example, enhancers, translation signals, and transcriptional and translation termination signals. Vectors that are used for stable transformation typically have a selectable marker which allows selection and maintenance of the transformed cells. In some embodiments, an origin of replication can be used to amplify the copy number of the vector in the cells.

Vectors can further comprise additional nucleic acids operably linked to a nucleic acid molecule (e.g., encoding a fusion polypeptide described herein), such as, for example, an epitope tag (e.g., for visualization, such as a fluorescent tag) and/or a tag for purification (e.g., a His tag).

Expression of biomolecular cargo and/or fusion polypeptides of the present disclosure can be controlled by any suitable promoter and/or enhancer known in the art. Suitable bacterial promoters are well known in the art and described herein below. Other suitable promoters for mammalian cells, yeast cells and insect cells are well known in the art. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bemoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 75:1441-1445 (1981)), the regulatory sequences of the metallothionein gene (Brinster et al., Nature 296:39-42 (1982)); prokaryotic expression vectors such as the b-lactamase promoter (Jay et al., Proc. Natl. Acad. Sci. USA 75:5543 (1981)) or the tac promoter (DeBoer et al., Proc. Natl. Acad. Sci. USA 50:21-25 (1983)); see also “Useful Proteins from Recombinant Bacteria”: in Scientific American 242:79-94 (1980)); plant expression vectors containing the nopaline synthetase promoter (Herrera-Estrella et al., Nature 505:209-213 (1984)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 510:1 15-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 515:115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7:1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al, Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al, Science 254:1372-1378 (1986))

In addition to the promoter, an expression vector typically contains a transcription unit or expression cassette that contains all the additional elements required for the expression of a biomolecular cargo and/or fusion polypeptide in cells. A typical expression cassette contains a promoter operably linked to the nucleic acid sequence encoding the biomolecular cargos and/or fusion polypeptides and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

### Engineered Vesicles (EngVs)

An EngV (e.g., a loaded EngV) produced in accordance with methods described herein or a plurality thereof are cell-derived vesicles comprising a membrane that encloses an internal space. EngVs can be or comprise any membrane-bound vesicles that have a smaller diameter than the cell from which the EngV is derived, such as exosomes, microvesicles, enveloped virus like particles, or enveloped gene delivery vectors (e.g., lentivirus).

Exosomes are small endosomal derived extracellular vesicles that are understood to contribute to many cellular processes, such as cell-cell communication, immune response, signal transduction, and antigen presentation. Typically, exosomes range in size from about 30 nm to about 300 nanometers (nm) in diameter. In some embodiments, exosomes (e.g., loaded exosomes) of the present disclosure have a diameter of from about 30 nm to about 300 nm, about 40 nm to about 300 nm, about 50 nm to about 300 nm, about 60 nm to about 300 nm, about 80 nm to about 300 nm, about 100 nm to about 300 nm, about 125 nm to about 300 nm, about 150 nm to about 300 nm, about 30 nm to about 250 nm, about 40 nm to about 250 nm, about 50 to about 250 nm, about 100 to about 250 nm, about 30 nm to about 200 nm, about 40 to about 200 nm, or about 50 to about 200 nm. In some embodiments, exosomes of the present disclosure have a diameter of about 30 nm, about 40 nm, about 50 nm, about 60 nm, about 70 nm, about 80 nm, about 90 nm, about 100 nm, about 120 nm, about 140 nm, about 160 nm, about 180 nm, about 200 nm, about 220 nm, about 240 nm, about 260 nm, about 280 nm, or about 300 nm.

Microvesicles are small plasma membrane derived vesicles generated by the outward budding and fission of the membrane vesicles from the cellular surface. It is currently understood that microvesicles also play important roles in cell-cell communication and typically range in size from about 30 nm to about 1,000 nm in diameter. In some embodiments, microvesicles (e.g., loaded microvesicles) of the present disclosure have a diameter of from about 30 nm to about 1,000 nm, about 50 nm to about 1,000 nm, about 100 nm to about 1,000 nm, about 200 nm to about 1,000 nm, about 300 nm to about 1,000 nm, about 400 nm to about 1,000 nm, about 500 nm to about 1,000 nm, about 600 nm to about 1,000 nm, about 30 nm to about 800 nm, about 50 nm to about 800 nm, about 100 nm to about 800 nm, about 200 nm to about 800 nm, about 300 nm to about 800 nm, about 30 nm to about 600 nm, or about 30 nm to about 400 nm. In some embodiments, microvesicles (e.g., loaded microvesicles) of the present disclosure have a diameter of about 20 nm, about 30 nm, about 40 nm, about 50 nm, about 100 nm, about 200 nm, about 300 nm, about 400 nm, about 500 nm, about 600 nm, about 700 nm, about 800 nm, about 900 nm, or about 1,000 nm.

Enveloped virus like particles (eVLPs) are nanostructures that comprise a membrane acquired from the host cell, called an envelope. In some embodiments, eVLPs comprise viral proteins on the outer surface. Some eVLPs are understood to comprise one or more glycoprotein spikes embedded in their envelope which can mediate binding, uptake, and fusion to recipient cells in order to achieve delivery of enclosed biomolecular cargo, such as a viral core (e.g., an AAV particle). In other usages, eVLPs are employed in vaccines to induce adaptive immune responses against the viral proteins. Typically, eVLPs have a diameter of greater than 100 nm. In some embodiments, eVLPs (e.g., loaded eVLPs) of the present disclosure have a diameter of from about 30 nm to about 300 nm, about 40 nm to about 300 nm, about 50 nm to about 300 nm, about 60 nm to about 300 nm, about 80 nm to about 300 nm, about 100 nm to about 300 nm, about 125 nm to about 300 nm, about 150 nm to about 300 nm, about 30 nm to about 250 nm, about 40 nm to about 250 nm, about 50 to about 250 nm, about 100 to about 250 nm, about 30 nm to about 200 nm, about 40 to about 200 nm, or about 50 to about 200 nm. In some embodiments, eVLPs of the present disclosure have a diameter of about 30 nm, about 40 nm, about 50 nm, about 60 nm, about 70 nm, about 80 nm, about 90 nm, about 100 nm, about 120 nm, about 140 nm, about 160 nm, about 180 nm, about 200 nm, about 220 nm, about 240 nm, about 260 nm, about 280 nm, or about 300 nm.

Certain enveloped gene delivery vectors, such as lentivirus, bud from the plasma membrane of cells in a manner similar to that of microvesicles. Typically, enveloped gene delivery vectors (e.g., loaded enveloped gene delivery vectors) are about 20 nm to about 150 nm in diameter. In some embodiments, enveloped gene delivery vectors (e.g., loaded enveloped gene delivery vectors) of the present disclosure have a diameter of from about 20 nm to about 200 nm, about 20 nm to 100 nm, about 75 nm to about 200 nm, about 100 nm to about 200 nm, about 125 nm to about 200 nm, about 150 nm to about 200 nm, about 20 nm to about 150 nm, about 20 nm to about 125 nm, about 50 nm to about 100 nm, about 75 nm to about 150 nm, about 75 nm to about 125 nm, or about 75 nm to about 100 nm. In some embodiments, enveloped gene delivery vectors of the present disclosure have a diameter of about 20 nm, about 30 nm, about 50 nm, about 75 nm, about 100 nm, about 125 nm, about 150 nm, about 175 nm, or about 200 nm.

In some embodiments, an EngV (e.g., a loaded EngV) of the present disclosure or a plurality thereof is or comprise exosomes. In some embodiments, an EngV (e.g., a loaded EngV) of the present disclosure or a plurality thereof is or comprise microvesicles. In some embodiments, an EngV (e.g., a loaded EngV) of the present disclosure or a plurality thereof is or comprise eVLPs. In some embodiments, an EngV (e.g., a loaded EngV) of the present disclosure or a plurality thereof is or comprise enveloped gene delivery vector. In some embodiments, plurality of EngVs of the present disclosure (e.g., loaded EngVs) comprise two or more of exosomes, microvesicles, eVLPs, and enveloped gene delivery vectors.

An EngV of the present disclosure or a plurality thereof can comprise one or more biomolecular cargos and/or fusion polypeptides (e.g., as described elsewhere herein) either in, on, or otherwise associated with an EngV, such that the biomolecular cargo and/or fusion polypeptide is encapsulated or associated with the EngV. Such EngVs are referred to herein as “loaded”. In some embodiments, an EngV is loaded with one, two, three, four, five six, seven, eight, nine, or ten different biomolecular cargos and/or fusion polypeptides.

A loaded EngV (e.g., produced in accordance with methods described herein) or a plurality thereof can include an increased concentration of biomolecular cargo and/or fusion polypeptide compared to an appropriate reference (e.g., a biomolecular cargo lacking a lipid tag, a biomolecular cargo with an unmodified transmembrane domain). In some embodiments, the concentration of biomolecular cargo and/or fusion polypeptide in a loaded EngV is a therapeutically effective amount of biomolecular cargo and/or fusion polypeptide. In some embodiments, the concentration of biomolecular cargo and/or fusion polypeptide in a loaded EngV is from about 2% to about 40% of total biomolecular cargo and/or fusion polypeptide present in the cell lysate, including from about 2% to about 35%, about 2% to about 30%, about 2% to about 25%, about 2% to about 20%, about 5% to about 40%, about 5% to about 35%, about 5% to about 30%, about 5% to about 25%, about 5% to about 20%, about 10% to about 40%, about 10% to about 35%, about 10% to about 30%, about 10% to about 25%, or about 10% to about 20%. In some embodiments, the concentration of biomolecular cargo and/or fusion polypeptide in a loaded EngV is from about 1 proteins per vesicle (ppv) to about 5,000 ppv, about 100 ppv to about 5,000 ppv, about 200 ppv to about 5,000 ppv, about 500 ppv to about 5,000 ppv, about 1,000 ppv to about 5,000 ppv, about 1 ppv to about 2,500 ppv, about 100 ppv to about 2,500 ppv, about 200 ppv to about 2,500 ppv, about 500 ppv to about 2,500 ppv, about 1,000 ppv to about 2,500 ppv, about 1 ppv to about 2,000 ppv, about 100 ppv to about 2,000 ppv, about 200 ppv to about 2,000 ppv, about 500 ppv to about 2,000 ppv, about 1,000 ppv to about 2,000 ppv, about 1 ppv to about 1,500 ppv, about 100 ppv to about 1,500 ppv, about 200 ppv to about 1,500 ppv, about 500 ppv to about 1,500 ppv, or about 1,000 ppv to about 1,500 ppv. In some embodiments, the concentration of biomolecular cargo and/or fusion polypeptide in a loaded EngV is from about 100 ppv to about 2,500 ppv.

A loaded EngV (e.g., produced in accordance with methods described herein) or a plurality thereof can include a (i) biomolecular cargo and/or (ii) a fusion polypeptide comprising a biomolecular cargo and one or more lipid tags with comparable functional activity to an appropriate reference (e.g., a biomolecular cargo lacking a lipid tag, a biomolecular cargo with an unmodified transmembrane domain). In some embodiments, functional activity of the biomolecular cargo part of a fusion polypeptide of the present disclosure is about 80%, about 85%, about 90%, about 95%, about 100%, about 105%, about 110%, or about 115%, about 120%, about 150%, about 200%, about 500%, about 1,000%, about 5,000%, or about 10,000% of the functional activity of an appropriate reference (e.g., a wild-type biomolecular cargo).

### Characterization

The present disclosure, among other things, provides technologies for characterizing an EngV (e.g., a loaded EngV) or a plurality thereof produced in accordance with technologies described herein. Characterization can be performed during and/or following methods described herein. In some embodiments, a particular loaded EngV preparation process may be modified or terminated in light of a characterization (e.g., if a particular preparation of a loaded EngV or a plurality thereof produced in accordance with the methods of the present disclosure fails to meet one or more specifications). Such characterization may involve assessment of one or more of yield, purity, loading, functional activity, or any combination thereof.

Characterization of yield of EngV (e.g., a loaded EngV) or a plurality thereof produced in accordance with technologies of the present disclosure can be assessed. A plurality of methods to quantify EngVs are known in the art, including, for example, immunoblotting of EngV-specific polypeptides, transmission electron microscopy, and nanoparticle tracking analysis (e.g., to quantify the number of EngVs in a sample as well as their size distribution). A plurality of EngV (e.g., loaded EngVs) can be quantified and yield can be determined by one or more of quantifying the number of EngVs in a sample or determining the yield of EngVs in sample before EngV isolation relative to after (also referred to as “recovery”). See, e.g., Hartjes TA. et al., Bioengineering (Basel). 2019 Jan. 16; 6 (1): 7).

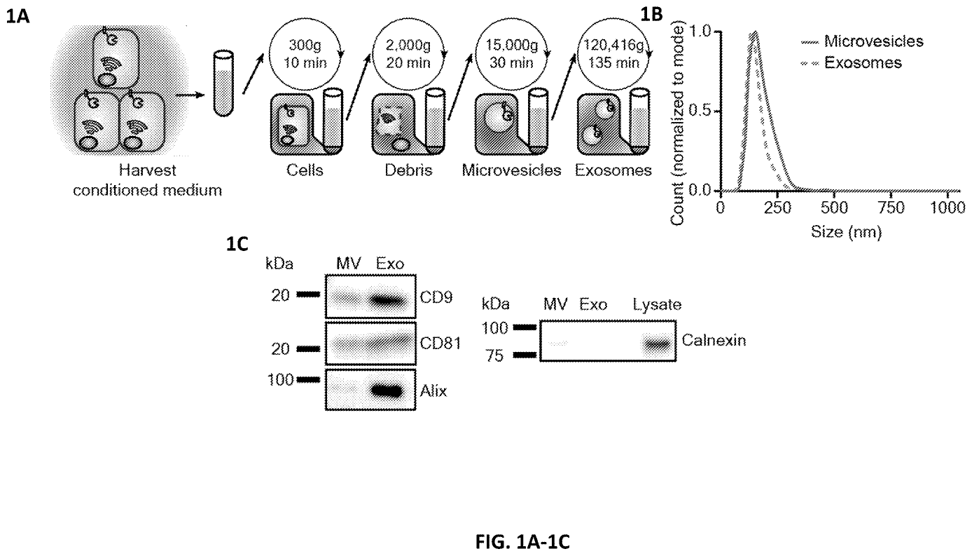

Characterization of purity of an EngV (e.g., a loaded EngV) or a plurality thereof produced in accordance with technologies of the present disclosure can be assessed. Purity of an EngV or a plurality thereof can be determined by a number of methods known in the art, such as measuring the amount of an EngV marker (e.g., by western blot), such as, for example, tetraspanins, CD9, CD63, and CD81, relative to a marker indicative to the amount of remaining undesired sample components. See, e.g., Dmitry Ter-Ovanesyan et al., eLife 10: e70725, FIG. 1C.

Characterization of the amount of fusion polypeptide in a loaded EngV or a plurality thereof (also referred to herein as “loading”) may be assessed. Loading can be assessed using a plurality of methods readily known and understood in the art. For example, loading may be assessed by immunoblot, mass spectrometry, or enzyme-linked immunoassay (ELISA). The amount of fusion polypeptide present in the engineered vesicle fraction can also be compared to the total amount of biomolecular cargo and/or fusion polypeptide present in the cell lysate. In some embodiments, a loaded EngV or a plurality thereof (e.g., produced in accordance with methods of the present disclosure) may be characterized to comprise a therapeutically effective amount of a biomolecular cargo and/or fusion polypeptide (e.g., a biomolecular cargo of a fusion polypeptide, such as a therapeutic polypeptide). In some embodiments, a loaded EngV or a plurality thereof may be characterized to have improved loaded (e.g., compared to an appropriate reference standard).

Characterization of functional activity of a biomolecular cargo (e.g., the biomolecular cargo of a fusion polypeptide) in a loaded EngV or plurality thereof (e.g., produced in accordance with methods described herein) can be assessed. Functional activity, such as, for example, signaling activity or enzymatic activity, of the biomolecular cargo can be assessed compared to an appropriate reference. An appropriate reference can be, for example, a wild-type biomolecular cargo (e.g., lacking a lipid tag, comprising an unmodified transmembrane domain) and/or a biomolecular cargo or fusion polypeptide of the present disclosure which was not loaded into an EngV.

A variety of methods are available to assess functional activity. For example, functional activity (e.g., signaling activity, enzymatic activity) can be assessed using an in vitro- or in vivo based activity assay. A functional activity assay can comprise, for example, measuring activation and/or inhibition of downstream signaling of a biomolecular cargo or enzymatic activity of a biomolecular cargo. A variety of methods are available to measure functional activity of a biomolecular cargo, such as, for example and without limitation, differential gene expression, protein expression, cleavage and/or conversion of certain substrate, and/or alterations in post-translational modifications. One of ordinary skill in the art reading the present disclosure would readily recognize that an appropriate assay to characterize functional activity would depend on the particular biomolecular cargo (e.g., biomolecular cargo included in the fusion polypeptide) and loaded into or on the EngV.

### Compositions

The present disclosure also provides compositions comprising a loaded EngV or a plurality thereof produced in accordance with technologies described herein.

Compositions comprising a loaded EngV or a plurality thereof of the present disclosure can be a pharmaceutical composition. Pharmaceutical compositions can comprise a loaded EngV or a plurality thereof and a pharmaceutically acceptable carrier. Appropriate pharmaceutically acceptable carriers, including but not limited to excipients and stabilizers, are known in the art. For example, a pharmaceutically acceptable carrier can be, without limitation, a buffer, an emulsifying agent, a dispersing agent, an isotonic agent, a wetting agent, a chelating agent, a sequestering agent, a pH buffering agent, a solubility enhancer, an antimicrobial agent, and/or an antioxidant. See, e.g., Remington's Pharmaceutical Sciences (1990) Mack Publishing Co., Easton, PA; Remington: The Science and Practice of Pharmacy, 21st Edition, A. R. Gennaro, Lippincott, Williams & Wilkins, Baltimore, MD, 2006.

Pharmaceutical compositions disclosed herein can comprise one or more pharmaceutically acceptable salts. Pharmaceutically acceptable salts are well known in the art. See, e.g., S. M. Berge, et al. J. Pharmaceutical Sciences, 66:1-19 (1977). Examples of pharmaceutically acceptable salts that can be included in pharmaceutical compositions of the present disclosure include, without limitation, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like.

Pharmaceutical compositions can be formulated such that they are suitable for administration to a subject (e.g., a human) in need of thereof. For example, pharmaceutical compositions can be formulated such that the pharmaceutical composition comprises a therapeutically effective amount of a fusion polypeptide of the present disclosure. A pharmaceutical composition may be formulated for any route of administration.

### Uses

Technologies of the present disclosure are useful in methods of loading an EngV or a plurality thereof with a biomolecular cargo and/or fusion polypeptide (e.g., as described herein). Such methods can provide a loaded EngV or plurality thereof that stably encapsulate or associate a biomolecular cargo and/or a fusion polypeptide into or on EngVs (e.g., in therapeutically effective amounts) without compromising the functional activity of the biomolecular cargos (e.g., of the fusion polypeptide) (e.g., by disrupting its structure and/or localization in the cell). As such, a loaded EngV or a plurality thereof produced in accordance with technologies of the present disclosure can be useful in delivering biomolecular cargo (e.g., as a part of a fusion polypeptide described herein) to a cell or a plurality of cells. Alternatively or additionally, technologies of the present disclosure can be utilized to load nucleic acids (e.g., mRNAs) or other molecules (e.g., small molecules, such as chemotherapeutic agents, gene therapy vectors, such as AAV, viral cores, and virus-like particle cores, a polypeptide that binds to the biomolecular cargo) into EngVs by loading biomolecular cargo (e.g., of a fusion polypeptide) that bind such molecules. As such, loading of the biomolecular cargo (e.g., of a fusion polypeptide) indirectly directs and loads such molecules into or on the EngV.

Methods of loading an EngV or a plurality thereof with a biomolecular cargo and/or fusion polypeptide as well as a loaded EngV or plurality thereof of the present disclosure can be used in, for example, biomedical research. Technologies of the present disclosure can facilitate studies to understand not only the dynamics of, for example, EngV generation and/or delivery, but also cell-cell communication.

Technologies of the present disclosure can facilitate improved loading of an EngV or a plurality thereof with a biomolecular cargo and/or a fusion polypeptide comprising any suitable biomolecular cargo known in the art, including, for example, a therapeutic polypeptide. As such, a loaded EngV or plurality thereof of the present disclosure can be useful delivering a therapeutic polypeptide (e.g., as a biomolecular cargo, as a part of a fusion polypeptide described herein) or a therapeutic nucleic acid to a subject in need thereof. Thus, technologies of the present disclosure can also be utilized in methods of treating a plurality of diseases and/or disorders in a subject in need thereof by facilitating delivery of therapeutic polypeptides and/or nucleic acids. Accordingly, in some aspects, the present disclosure provides methods of treating a disease and/or disorder in a subject in need thereof comprising administering a loaded EngV or a plurality thereof or a composition of the present disclosure to a subject in need thereof.

A subject in need thereof can have, for example, a disease and/or disorder associated with a deficiency of a particular polypeptide (e.g., certain lysosomal storage diseases), a genetic disease and/or disorder, an infectious disease or a disposition to becoming infecting with an infectious disease (e.g., a bacterial infection, a viral infection), an autoimmune disease, and/or cancer.

A loaded EngV or a plurality thereof or compositions of the present disclosure can be administered to a subject with a disease and/or disorder associated with deficiency of a particular polypeptide to deliver the particular deficient polypeptide (or nucleic acid encoding the particular deficient polypeptide) to the subject, thereby treating the disease and/or disorder.

Genetic diseases and/or disorders may be treated by administering a loaded EngV or a plurality thereof or compositions of the present disclosure, wherein the EngV is loaded with a gene-editing polypeptide for use in gene therapy. Such gene-editing polypeptides include, for example and without limitation, zinc finger nucleases, transcription activator-like effector nucleases (TALENs), and/or Clustered Regularly Interspaced Short Palindromic Repeats (CRISPR)-associated protein (e.g., Cas9, Cas12a, Cas12b) (e.g., in complex with a guide RNA). Use of gene-editing polypeptides to modify disease and/or disorder causing mutations and/or variants is readily understood by those of ordinary skill in the art. See, e.g., Gaj T. et al., Cold Spring Harb Perspect Biol. 2016 Dec. 1; 8 (12): a023754.

A loaded EngV or a plurality thereof or compositions of the present disclosure can also be utilized to deliver an antigen to a subject at a disposition to becoming infected with an infectious disease, such as a bacterial infection or a viral infection (e.g., in a method of vaccination). Infectious diseases may also be treated with a loaded EngV or a plurality thereof of the present disclosure by loading, for example, a biomolecular cargo and/or fusion polypeptide comprising an antibody as biomolecular cargo that can target the particular infectious disease and/or reduce symptoms associated therewith.

Cancer may be treated by administering a loaded EngV or a plurality thereof or compositions of the present disclosure, wherein the EngV is loaded with a fusion polypeptide comprising a biomolecular cargo that can, for example, decrease cancer cell proliferation, increase cancer cell apoptosis, enhance an immune response to the cancer cells and/or increase cancer cell targeting of the subject's immune response. Alternatively or additionally, cancer may be treated by administering a loaded EnvG or a plurality thereof or compositions of the present disclosure, wherein the EngV is loaded with a therapeutic nucleic acid or other molecule (e.g., chemotherapeutic agent) that bound to a biomolecular cargo and/or fusion polypeptide of the disclosure, thereby directing and loading the therapeutic nucleic acid or other molecule into or on the EV.

A loaded EngV, plurality thereof, or composition of the present disclosure may be administered by to a subject in need thereof, for example, intravenous, intramuscular, intraarterial, subcutaneous, intratumoral, intrathecal, intraosseous, or intraperitoneal administration.

### EXAMPLES

### Example 1: Materials and Methods

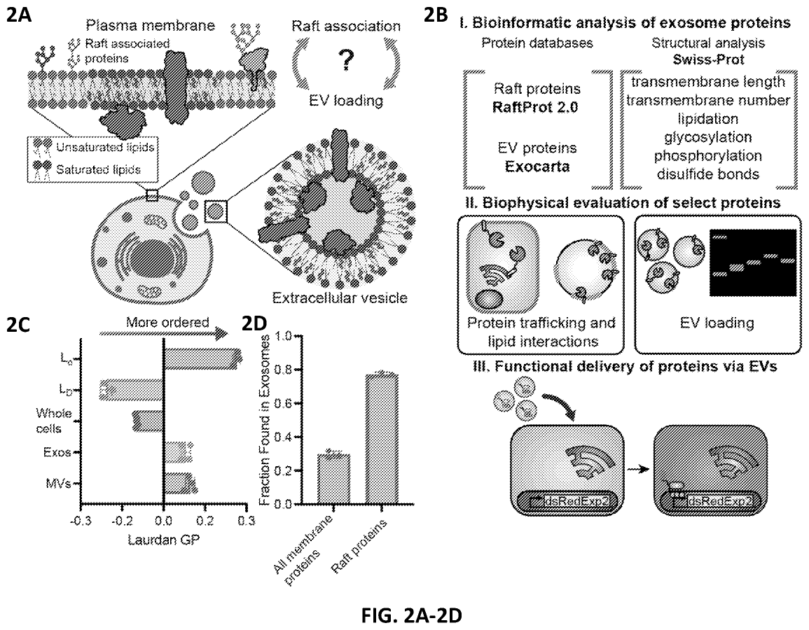

Bioinformatics: To perform the bioinformatic analysis, a list of raft proteins populating the list of human raft associated proteins in RaftProt 2.0 with sequence and structural information from Swiss-Prot was generated. Similarly, a database of human EV proteins was created by pulling protein information from Swiss-Prot for proteins found in Exocarta, an EV database. To determine the probability of a raft protein or membrane associated protein to be found in an EV, 100 human proteins were randomly selected from either RaftProt 2.0 or a list of all human membrane-associated proteins in Swiss-Prot, and the fraction of each subset of proteins found in Exocarta was calculated, reported as the frequency found in EVs (FIG. 2D). By utilizing this database of annotated raft, EV, and membrane associated proteins, the average transmembrane domain length, frequency and number of lipidation sites (myristylation, palmitoylation, prenylation), glycosylation, phosphorylation, and the presence of disulfide bonds were determined. Transmembrane domain length was estimated by multiplying the number of amino acids within the transmembrane domains by 0.15 nm. Transmembrane domain length for multi-pass transmembrane domain proteins is reported as the average length for all transmembrane domains. The number of proteins in each category are listed in Table 8.

Cloning and DNA preparation: Plasmid cloning was performed using standard PCR and restriction enzyme cloning techniques. Genes were typically ordered as gBlocks from Twist Biosciences, and primers for cloning were ordered from Integrated DNA Technologies. Genes encoding proteins used for protein localization were cloned into the Twist Biosciences CMV mammalian expression vector (CMV Puro). A gBlock encoding the p65 ZF6 SynTF was ordered from Twist and cloned into plasmids with lipidation tags. Plasmids encoding fluorescent proteins were generally cloned into a modified pcDNA3.1 backbone (Thermo Fisher #87020b). DNA sequences and sources of key sequences are in Tables 3-5. The BxB1 recombinase expression vector plasmid was kindly gifted. DsRed-Express2 was obtained by site directed mutagenesis of pDsRed2-N1, which was kindly gifted. BlastR was sourced from lenti dCAS-VP64_Blast, which was kindly gifted. Enzymes and buffers required for PCR and cloning were purchased from Thermo Fisher and/or NEB. Plasmid DNA was prepared by growing 100 mL of Top10 E. coli overnight with the appropriate selective antibiotic and then purifying using the PureLink Plasmid Midiprep Kit (Thermo Fisher), ZymoPURE II Midiprep Kit (Zymo Research) or polyethylene glycol precipitation.

TABLE 3
Protein source and sequences for proteins used in this study.

| | PROTEIN | |
|---|---|---|
| TAG | SOURCE | PROTEIN SEQUENCE |
| LAT WT | LAT | MEEAILVPCVLGLLLLPILAMLMALCVHCHRLP (SEQ |
| | | ID NO: 1) |
| LAT C26A | LAT-modified | MEEAILVPCVLGLLLLPILAMLMALAVHCHRLP (SEQ |
| | | ID NO: 2) |
| LAT | LAT-modified | MEEAILVPCVLGLAMLMALCVHCHRLP (SEQ ID NO: 3) |
| DCORE | | |
| LAT HIGH | LAT-modified | MEEAILVLLLLLLLLLPLLLLLLLLCVHCHRLP (SEQ ID |
| ASA | | NO: 4) |
| 24Å TM4 | de novo | MGSTRTEIIRELERSLREQRVLAIFLLALLIVLLWLLQQL |
| | designed | KELLRELERLQREGSSDEDVRELLREIKELVENIVYLVII |
| | | IMVLVLVIIALARTQKYLVEELKRQD (SEQ ID NO: 5) |
| 32Å TM4 | de novo | MGSTRTEIITRLSFSLLLQLVLAIFLLALLIVLLWLLQQL |
| | designed | KELLRELERLQREGSSDEDVRELLREIKELVENIVYLVII |
| | | IMVLVLVIIALAVLQMYLVRELKRQD (SEQ ID NO: 6) |
| 40Å TM4 | de novo | MGSTRTEIITRLSFSLLLQLVLAIFLLALLIVLLVLLIYLK |
| | designed | ELLRELERLQREGSSDEDVRELLREIKWLVIVIVALVIII |
| | | MVLVLVIIALAVLQMYLVRELKRQD (SEQ ID NO: 7) |
| 24Å TM12 | de novo | MTENEIRKLRKLLRIAMFLLVFLLIWTWISLETSKTDDD |
| | designed | PSAQSEALVAMSLMLIAASLLIIAKSKLMKSRNG (SEQ |
| | | ID NO: 8) |
| 40Å TM12 | de novo | MTKKIIMVLILLLIIAMLLLVFLLIATVVSLWWSWTDD |
| | designed | DPSAISEALVAMSLMLIAASLLIIAISKLLKSKNG (SEQ |
| | | ID NO: 9) |
| M | Src | MGSSKSKPKDPSQRRNNNNGPVAT (SEQ ID NO: 10) |
| PM | Lyn | MGCIKSKRKDKDLELKLRILQSTVPRARDPPVAT (SEQ |
| | | ID NO: 11) |
| G | K-Ras | FRSDGKKKKKKSKTKCQLL (SEQ ID NO: 12) |
| PPF | Paralemmin-1 | CKCCSIM (SEQ ID NO: 13) |

TABLE 4
DNA sequences for protein loading studies.

| Construct | DNA Sequence |
|---|---|
| WT LAT HALO | ATGGAGGAGGCCATCCTGGTCCCCTGCGTGCTGGGGCTCCTGCTGCTGCCCATCCTGGCCATGTTGAT |
| | GGCACTGTGTGTGCACTGCCACAGACTGCCAGGCTCCGGATCCGGCGGCTCCGAAATCGGTACTGGCT |
| | TTCCATTCGACCCCCATTATGTGGAAGTCCTGGGCGAGCGCATGCACTACGTCGATGTTGGTCCGCGCG |
| | ATGGCACCCCTGTGCTGTTCCTGCACGGTAACCCGACCTCCTCCTACGTGTGGCGCAACATCATCCCGC |
| | ATGTTGCACCGACCCATCGCTGCATTGCTCCAGACCTGATCGGTATGGGCAAATCCGACAAACCAGACC |
| | TGGGTTATTTCTTCGACGACCACGTCCGCTTCATGGATGCCTTCATCGAAGCCCTGGGTCTGGAAGAGG |
| | TCGTCCTGGTCATTCACGACTGGGGCTCCGCTCTGGGTTTCCACTGGGCCAAGCGCAATCCAGAGCGC |
| | GTCAAAGGTATTGCATTTATGGAGTTCATCCGCCCTATCCCGACCTGGGACGAATGGCCAGAATTTGCCC |
| | GCGAGACCTTCCAGGCCTTCCGCACCACCGACGTCGGCCGCAAGCTGATCATCGATCAGAACGTTTTTA |
| | TCGAGGGTACGCTGCCGATGGGTGTCGTCCGCCCGCTGACTGAAGTCGAGATGGACCATTACCGCGAG |
| | CCGTTCCTGAATCCTGTTGACCGCGAGCCACTGTGGCGCTTCCCAAACGAGCTGCCAATCGCCGGTGAG |
| | CCAGCGAACATCGTCGCGCTGGTCGAAGAATACATGGACTGGCTGCACCAGTCCCCTGTCCCGAAGCT |
| | GCTGTTCTGGGGCACCCCAGGCGTTCTGATCCCACCGGCCGAAGCCGCTCGCCTGGCCAAAAGCCTGC |
| | CTAACTGCAAGGCTGTGGACATCGGCCCGGGTCTGAATCTGCTGCAAGAAGACAACCCGGACCTGATCG |
| | GCAGCGAGATCGCGCGCTGGCTGTCTACTCTGGAGATTTCCGGCTCCGAATTCGATTACAAGGACCACG |
| | ATGGCGACTATAAGGATCACGACATCGACTACAAGGACGATGACGACAAGTGA (SEQ ID NO: 14) |
| LAT C26A HALO | ATGGAGGAGGCCATCCTGGTCCCCTGCGTGCTGGGGCTCCTGCTGCTGCCCATCCTGGCCATGTTGAT |
| | GGCACTGgccGTGCACTGCCACAGACTGCCAGGATCCGGCGGCTCCGAAATCGGTACTGGCTTTCCATT |
| | CGACCCCCATTATGTGGAAGTCCTGGGCGAGCGCATGCACTACGTCGATGTTGGTCCGCGCGATGGCA |
| | CCCCTGTGCTGTTCCTGCACGGTAACCCGACCTCCTCCTACGTGTGGCGCAACATCATCCCGCATGTTG |
| | CACCGACCCATCGCTGCATTGCTCCAGACCTGATCGGTATGGGCAAATCCGACAAACCAGACCTGGGTT |
| | ATTTCTTCGACGACCACGTCCGCTTCATGGATGCCTTCATCGAAGCCCTGGGTCTGGAAGAGGTCGTCC |
| | TGGTCATTCACGACTGGGGCTCCGCTCTGGGTTTCCACTGGGCCAAGCGCAATCCAGAGCGCGTCAAA |
| | GGTATTGCATTTATGGAGTTCATCCGCCCTATCCCGACCTGGGACGAATGGCCAGAATTTGCCCGCGAG |
| | ACCTTCCAGGCCTTCCGCACCACCGACGTCGGCCGCAAGCTGATCATCGATCAGAACGTTTTTATCGAG |
| | GGTACGCTGCCGATGGGTGTCGTCCGCCCGCTGACTGAAGTCGAGATGGACCATTACCGCGAGCCGTT |
| | CCTGAATCCTGTTGACCGCGAGCCACTGTGGCGCTTCCCAAACGAGCTGCCAATCGCCGGTGAGCCAG |
| | CGAACATCGTCGCGCTGGTCGAAGAATACATGGACTGGCTGCACCAGTCCCCTGTCCCGAAGCTGCTGT |
| | TCTGGGGCACCCCAGGCGTTCTGATCCCACCGGCCGAAGCCGCTCGCCTGGCCAAAAGCCTGCCTAAC |
| | TGCAAGGCTGTGGACATCGGCCCGGGTCTGAATCTGCTGCAAGAAGACAACCCGGACCTGATCGGCAG |
| | CGAGATCGCGCGCTGGCTGTCTACTCTGGAGATTTCCGGCTCCGAATTCGATTACAAGGACCACGATGG |
| | CGACTATAAGGATCACGACATCGACTACAAGGACGATGACGACAAGTGA (SEQ ID NO: 15) |
| LAT dCore | ATGGAGGAGGCCATCCTGGTCCCCTGCGTGCTGGGGCTCGCCATGTTGATGGCACTGTGTGTGCACTG |
| HALO | CCACAGACTGCCAGGATCCGGCGGCTCCGAAATCGGTACTGGCTTTCCATTCGACCCCCATTATGTGGA |
| | AGTCCTGGGCGAGCGCATGCACTACGTCGATGTTGGTCCGCGCGATGGCACCCCTGTGCTGTTCCTGC |
| | ACGGTAACCCGACCTCCTCCTACGTGTGGCGCAACATCATCCCGCATGTTGCACCGACCCATCGCTGCA |
| | TTGCTCCAGACCTGATCGGTATGGGCAAATCCGACAAACCAGACCTGGGTTATTTCTTCGACGACCACG |
| | TCCGCTTCATGGATGCCTTCATCGAAGCCCTGGGTCTGGAAGAGGTCGTCCTGGTCATTCACGACTGGG |
| | GCTCCGCTCTGGGTTTCCACTGGGCCAAGCGCAATCCAGAGCGCGTCAAAGGTATTGCATTTATGGAGT |
| | TCATCCGCCCTATCCCGACCTGGGACGAATGGCCAGAATTTGCCCGCGAGACCTTCCAGGCCTTCCGCA |
| | CCACCGACGTCGGCCGCAAGCTGATCATCGATCAGAACGTTTTTATCGAGGGTACGCTGCCGATGGGTG |
| | TCGTCCGCCCGCTGACTGAAGTCGAGATGGACCATTACCGCGAGCCGTTCCTGAATCCTGTTGACCGCG |
| | AGCCACTGTGGCGCTTCCCAAACGAGCTGCCAATCGCCGGTGAGCCAGCGAACATCGTCGCGCTGGTC |
| | GAAGAATACATGGACTGGCTGCACCAGTCCCCTGTCCCGAAGCTGCTGTTCTGGGGCACCCCAGGCGT |
| | TCTGATCCCACCGGCCGAAGCCGCTCGCCTGGCCAAAAGCCTGCCTAACTGCAAGGCTGTGGACATCG |
| | GCCCGGGTCTGAATCTGCTGCAAGAAGACAACCCGGACCTGATCGGCAGCGAGATCGCGCGCTGGCTG |
| | TCTACTCTGGAGATTTCCGGCTCCGAATTCGATTACAAGGACCACGATGGCGACTATAAGGATCACGAC |
| | ATCGACTACAAGGACGATGACGACAAGTGA (SEQ ID NO: 16) |
| LAT High ASA | ATGGAAGAGGCCATCCTGGTTCTGCTGCTGCTCCTTCTGCTTCTGCTGCCTCTTCTCTTGCTCCTCCTGC |
| HALO | TGCTGTGCGTGCACTGTCACAGATTGCCTGGATCCGGCGGCTCCGAAATCGGTACTGGCTTTCCATTCG |
| | ACCCCCATTATGTGGAAGTCCTGGGCGAGCGCATGCACTACGTCGATGTTGGTCCGCGCGATGGCACC |
| | CCTGTGCTGTTCCTGCACGGTAACCCGACCTCCTCCTACGTGTGGCGCAACATCATCCCGCATGTTGCA |
| | CCGACCCATCGCTGCATTGCTCCAGACCTGATCGGTATGGGCAAATCCGACAAACCAGACCTGGGTTAT |
| | TTCTTCGACGACCACGTCCGCTTCATGGATGCCTTCATCGAAGCCCTGGGTCTGGAAGAGGTCGTCCTG |
| | GTCATTCACGACTGGGGCTCCGCTCTGGGTTTCCACTGGGCCAAGCGCAATCCAGAGCGCGTCAAAGG |
| | TATTGCATTTATGGAGTTCATCCGCCCTATCCCGACCTGGGACGAATGGCCAGAATTTGCCCGCGAGAC |
| | CTTCCAGGCCTTCCGCACCACCGACGTCGGCCGCAAGCTGATCATCGATCAGAACGTTTTTATCGAGGG |
| | TACGCTGCCGATGGGTGTCGTCCGCCCGCTGACTGAAGTCGAGATGGACCATTACCGCGAGCCGTTCC |
| | TGAATCCTGTTGACCGCGAGCCACTGTGGCGCTTCCCAAACGAGCTGCCAATCGCCGGTGAGCCAGCG |
| | AACATCGTCGCGCTGGTCGAAGAATACATGGACTGGCTGCACCAGTCCCCTGTCCCGAAGCTGCTGTTC |
| | TGGGGCACCCCAGGCGTTCTGATCCCACCGGCCGAAGCCGCTCGCCTGGCCAAAAGCCTGCCTAACTG |
| | CAAGGCTGTGGACATCGGCCCGGGTCTGAATCTGCTGCAAGAAGACAACCCGGACCTGATCGGCAGCG |
| | AGATCGCGCGCTGGCTGTCTACTCTGGAGATTTCCGGCTCCGAATTCGATTACAAGGACCACGATGGCG |
| | ACTATAAGGATCACGACATCGACTACAAGGACGATGACGACAAGTGA (SEQ ID NO: 17) |
| 24 Å TM4 RFP | ATGGGCAGCACCAGAACCGAGATCATCAGAGAGCTGGAAAGAAGCCTGCGCGAGCAGAGAGTGCTGGC |
| | CATTTTTCTGCTGGCCCTGCTGATCGTGCTGCTGTGGCTGCTGCAACAGCTGAAAGAGCTGCTGAGAGA |
| | ACTGGAACGGCTGCAGAGAGAGGGCAGCTCTGACGAAGATGTGCGGGAACTGCTGCGCGAGATCAAAG |
| | AACTGGTGGAAAACATCGTGTACCTGGTTATCATCATCATGGTGCTGGTGCTCGTGATCATTGCCCTGGC |
| | CAGAACACAGAAGTACCTGGTCGAGGAACTGAAGCGGCAGGACGGATCCGGCGGCTCCGAAATCGGTA |
| | CTGGCTTTCCATTCGACCCCCATTATGTGGAAGTCCTGGGCGAGCGCATGCACTACGTCGATGTTGGTC |
| | CGCGCGATGGCACCCCTGTGCTGTTCCTGCACGGTAACCCGACCTCCTCCTACGTGTGGCGCAACATC |
| | ATCCCGCATGTTGCACCGACCCATCGCTGCATTGCTCCAGACCTGATCGGTATGGGCAAATCCGACAAA |
| | CCAGACCTGGGTTATTTCTTCGACGACCACGTCCGCTTCATGGATGCCTTCATCGAAGCCCTGGGTCTG |
| | GAAGAGGTCGTCCTGGTCATTCACGACTGGGGCTCCGCTCTGGGTTTCCACTGGGCCAAGCGCAATCC |
| | AGAGCGCGTCAAAGGTATTGCATTTATGGAGTTCATCCGCCCTATCCCGACCTGGGACGAATGGCCAGA |
| | ATTTGCCCGCGAGACCTTCCAGGCCTTCCGCACCACCGACGTCGGCCGCAAGCTGATCATCGATCAGA |
| | ACGTTTTTATCGAGGGTACGCTGCCGATGGGTGTCGTCCGCCCGCTGACTGAAGTCGAGATGGACCATT |
| | ACCGCGAGCCGTTCCTGAATCCTGTTGACCGCGAGCCACTGTGGCGCTTCCCAAACGAGCTGCCAATC |
| | GCCGGTGAGCCAGCGAACATCGTCGCGCTGGTCGAAGAATACATGGACTGGCTGCACCAGTCCCCTGT |
| | CCCGAAGCTGCTGTTCTGGGGCACCCCAGGCGTTCTGATCCCACCGGCCGAAGCCGCTCGCCTGGCCA |
| | AAAGCCTGCCTAACTGCAAGGCTGTGGACATCGGCCCGGGTCTGAATCTGCTGCAAGAAGACAACCCG |
| | GACCTGATCGGCAGCGAGATCGCGCGCTGGCTGTCTACTCTGGAGATTTCCGGCTCCGAATTCGATTAC |
| | AAGGACCACGATGGCGACTATAAGGATCACGACATCGACTACAAGGACGATGACGACAAGTGA (SEQ ID |
| | NO: 18) |
| 32 Å TM4 RFP | ATGGGCAGCACCAGAACCGAGATCATCACCCGGCTGAGCTTCAGCCTGCTGCTGCAACTGGTGCTGGC |
| | TATCTTTCTGCTGGCCCTGCTGATCGTGCTGCTGTGGCTGCTTCAGCAGCTGAAAGAGCTGCTGAGAGA |
| | GCTGGAACGGCTGCAGAGAGAGGGAAGCTCTGACGAGGATGTGCGGGAACTGCTGCGCGAGATCAAA |
| | GAACTGGTGGAAAACATCGTGTACCTGGTTATCATCATCATGGTGCTGGTGCTCGTGATCATTGCCCTGG |
| | CCGTGCTGCAGATGTACCTCGTCAGGGAACTGAAGCGGCAGGACGGATCCGGCGGCTCCATGGCCTCC |
| | TCCGAGGACGTCATCAAGGAGTTCATGCGCTTCAAGGTGCGCATGGAGGGCTCCGTGAACGGCCACGA |
| | GTTCGAGATCGAGGGCGAGGGCGAGGGCCGCCCCTACGAGGGCACCCAGACCGCCAAGCTGAAGGTG |
| | ACCAAGGGCGGCCCCCTGCCCTTCGCCTGGGACATCCTGTCCCCTCAGTTCCAGTACGGCTCCAAGGC |
| | CTACGTGAAGCACCCCGCCGACATCCCCGACTACTTGAAGCTGTCCTTCCCCGAGGGCTTCAAGTGGGA |
| | GCGCGTGATGAACTTCGAGGACGGCGGCGTGGTGACCGTGACCCAGGACTCCTCCCTGCAGGACGGC |
| | GAGTTCATCTACAAGGTGAAGCTGCGCGGCACCAACTTCCCCTCCGACGGCCCCGTAATGCAGAAGAA |
| | GACCATGGGCTGGGAGGCCTCCACCGAGCGGATGTACCCCGAGGACGGCGCCCTGAAGGGCGAGATC |
| | AAGATGAGGCTGAAGCTGAAGGACGGCGGCCACTACGACGCCGAGGTCAAGACCACCTACATGGCCAA |
| | GAAGCCCGTGCAGCTGCCCGGCGCCTACAAGACCGACATCAAGCTGGACATCACCTCCCACAACGAGG |
| | ACTACACCATCGTGGAACAGTACGAGCGCGCCGAGGGCCGCCACTCCACCGGCGCCTCCGGCTCCGA |
| | ATTCGATTACAAGGACCACGATGGCGACTATAAGGATCACGACATCGACTACAAGGACGATGACGACAA |
| | GTGA (SEQ ID NO: 19) |
| 40 Å TM4 RFP | ATGGGCAGCACCAGAACCGAGATCATCACCCGGCTGAGCTTCAGCCTGCTGCTGCAACTGGTGCTGGC |
| | TATCTTTCTGCTGGCCCTGCTGATCGTGCTGCTGGTGCTCCTGATCTACCTGAAAGAGCTGCTGAGAGA |
| | GCTGGAACGGCTGCAGAGAGAGGGAAGCTCTGACGAGGATGTGCGAGAACTGCTGCGCGAGATCAAGT |
| | GGCTGGTCATCGTGATCGTGGCCCTGGTTATCATCATCATGGTGCTGGTCCTGGTCATCATTGCCCTGG |
| | CCGTGCTGCAGATGTACCTCGTGCGGGAACTGAAGAGACAGGACGGATCCGGCGGCTCCATGGCCTCC |
| | TCCGAGGACGTCATCAAGGAGTTCATGCGCTTCAAGGTGCGCATGGAGGGCTCCGTGAACGGCCACGA |
| | GTTCGAGATCGAGGGCGAGGGCGAGGGCCGCCCCTACGAGGGCACCCAGACCGCCAAGCTGAAGGTG |
| | ACCAAGGGCGGCCCCCTGCCCTTCGCCTGGGACATCCTGTCCCCTCAGTTCCAGTACGGCTCCAAGGC |
| | CTACGTGAAGCACCCCGCCGACATCCCCGACTACTTGAAGCTGTCCTTCCCCGAGGGCTTCAAGTGGGA |
| | GCGCGTGATGAACTTCGAGGACGGCGGCGTGGTGACCGTGACCCAGGACTCCTCCCTGCAGGACGGC |
| | GAGTTCATCTACAAGGTGAAGCTGCGCGGCACCAACTTCCCCTCCGACGGCCCCGTAATGCAGAAGAA |
| | GACCATGGGCTGGGAGGCCTCCACCGAGCGGATGTACCCCGAGGACGGCGCCCTGAAGGGCGAGATC |
| | AAGATGAGGCTGAAGCTGAAGGACGGCGGCCACTACGACGCCGAGGTCAAGACCACCTACATGGCCAA |
| | GAAGCCCGTGCAGCTGCCCGGCGCCTACAAGACCGACATCAAGCTGGACATCACCTCCCACAACGAGG |
| | ACTACACCATCGTGGAACAGTACGAGCGCGCCGAGGGCCGCCACTCCACCGGCGCCTCCGGCTCCGA |
| | ATTCGATTACAAGGACCACGATGGCGACTATAAGGATCACGACATCGACTACAAGGACGATGACGACAA |
| | GTGA (SEQ ID NO: 20) |
| 24 Å TM12 RFP | ATGACCGAGAACGAGATCCGGAAGCTGAGAAAGCTGCTGCGGATCGCTATGTTCCTGCTGGTGTTTCTG |
| | CTGATCTGGACCTGGATCAGCCTGGAAACCAGCAAGACCGACGACGACCCTAGCGCTCAGTCTGAAGC |
| | TCTGGTGGCCATGAGCCTGATGCTGATTGCCGCCAGCCTGCTGATCATTGCCAAGAGCAAGCTGATGAA |
| | GTCCCGGAACGGAGGATCCGGCGGCTCCGAAATCGGTACTGGCTTTCCATTCGACCCCCATTATGTGGA |
| | AGTCCTGGGCGAGCGCATGCACTACGTCGATGTTGGTCCGCGCGATGGCACCCCTGTGCTGTTCCTGC |
| | ACGGTAACCCGACCTCCTCCTACGTGTGGCGCAACATCATCCCGCATGTTGCACCGACCCATCGCTGCA |
| | TTGCTCCAGACCTGATCGGTATGGGCAAATCCGACAAACCAGACCTGGGTTATTTCTTCGACGACCACG |
| | TCCGCTTCATGGATGCCTTCATCGAAGCCCTGGGTCTGGAAGAGGTCGTCCTGGTCATTCACGACTGGG |
| | GCTCCGCTCTGGGTTTCCACTGGGCCAAGCGCAATCCAGAGCGCGTCAAAGGTATTGCATTTATGGAGT |
| | TCATCCGCCCTATCCCGACCTGGGACGAATGGCCAGAATTTGCCCGCGAGACCTTCCAGGCCTTCCGCA |
| | CCACCGACGTCGGCCGCAAGCTGATCATCGATCAGAACGTTTTTATCGAGGGTACGCTGCCGATGGGTG |
| | TCGTCCGCCCGCTGACTGAAGTCGAGATGGACCATTACCGCGAGCCGTTCCTGAATCCTGTTGACCGCG |
| | AGCCACTGTGGCGCTTCCCAAACGAGCTGCCAATCGCCGGTGAGCCAGCGAACATCGTCGCGCTGGTC |
| | GAAGAATACATGGACTGGCTGCACCAGTCCCCTGTCCCGAAGCTGCTGTTCTGGGGCACCCCAGGCGT |
| | TCTGATCCCACCGGCCGAAGCCGCTCGCCTGGCCAAAAGCCTGCCTAACTGCAAGGCTGTGGACATCG |
| | GCCCGGGTCTGAATCTGCTGCAAGAAGACAACCCGGACCTGATCGGCAGCGAGATCGCGCGCTGGCTG |
| | TCTACTCTGGAGATTTCCGGCTCCGAATTCGATTACAAGGACCACGATGGCGACTATAAGGATCACGAC |
| | ATCGACTACAAGGACGATGACGACAAGTGA (SEQ ID NO: 21) |
| 40 Å TM12 RFP | ATGACCAAGAAAATCATCATGGTGCTGATCCTGCTGCTGATCATTGCTATGCTGCTGCTGGTGTTCCTGC |
| | TGATCGCCACCGTGGTTTCCCTGTGGTGGTCCTGGACCGACGATGATCCTAGCGCCATTTCTGAAGCCC |
| | TGGTGGCCATGAGCCTGATGCTGATTGCTGCCAGCCTGCTGATTATCGCCATCAGCAAGCTGCTGAAGT |
| | CCAAGAACGGCGGATCCGGCGGCTCCATGGCCTCCTCCGAGGACGTCATCAAGGAGTTCATGCGCTTC |
| | AAGGTGCGCATGGAGGGCTCCGTGAACGGCCACGAGTTCGAGATCGAGGGCGAGGGCGAGGGCCGC |
| | CCCTACGAGGGCACCCAGACCGCCAAGCTGAAGGTGACCAAGGGCGGCCCCCTGCCCTTCGCCTGGG |
| | ACATCCTGTCCCCTCAGTTCCAGTACGGCTCCAAGGCCTACGTGAAGCACCCCGCCGACATCCCCGACT |
| | ACTTGAAGCTGTCCTTCCCCGAGGGCTTCAAGTGGGAGCGCGTGATGAACTTCGAGGACGGCGGCGTG |
| | GTGACCGTGACCCAGGACTCCTCCCTGCAGGACGGCGAGTTCATCTACAAGGTGAAGCTGCGCGGCAC |
| | CAACTTCCCCTCCGACGGCCCCGTAATGCAGAAGAAGACCATGGGCTGGGAGGCCTCCACCGAGCGGA |
| | TGTACCCCGAGGACGGCGCCCTGAAGGGCGAGATCAAGATGAGGCTGAAGCTGAAGGACGGCGGCCA |
| | CTACGACGCCGAGGTCAAGACCACCTACATGGCCAAGAAGCCCGTGCAGCTGCCCGGCGCCTACAAGA |
| | CCGACATCAAGCTGGACATCACCTCCCACAACGAGGACTACACCATCGTGGAACAGTACGAGCGCGCC |
| | GAGGGCCGCCACTCCACCGGCGCCTCCGGCTCCGAATTCGATTACAAGGACCACGATGGCGACTATAA |
| | GGATCACGACATCGACTACAAGGACGATGACGACAAGTGA (SEQ ID NO: 22) |
| Soluble HALO | ATGGAATTCGGCGGCTCCGAAATCGGTACTGGCTTTCCATTCGACCCCCATTATGTGGAAGTCCTGGGC |
| | GAGCGCATGCACTACGTCGATGTTGGTCCGCGCGATGGCACCCCTGTGCTGTTCCTGCACGGTAACCC |
| | GACCTCCTCCTACGTGTGGCGCAACATCATCCCGCATGTTGCACCGACCCATCGCTGCATTGCTCCAGA |
| | CCTGATCGGTATGGGCAAATCCGACAAACCAGACCTGGGTTATTTCTTCGACGACCACGTCCGCTTCAT |
| | GGATGCCTTCATCGAAGCCCTGGGTCTGGAAGAGGTCGTCCTGGTCATTCACGACTGGGGCTCCGCTC |
| | TGGGTTTCCACTGGGCCAAGCGCAATCCAGAGCGCGTCAAAGGTATTGCATTTATGGAGTTCATCCGCC |
| | CTATCCCGACCTGGGACGAATGGCCAGAATTTGCCCGCGAGACCTTCCAGGCCTTCCGCACCACCGAC |
| | GTCGGCCGCAAGCTGATCATCGATCAGAACGTTTTTATCGAGGGTACGCTGCCGATGGGTGTCGTCCGC |
| | CCGCTGACTGAAGTCGAGATGGACCATTACCGCGAGCCGTTCCTGAATCCTGTTGACCGCGAGCCACTG |
| | TGGCGCTTCCCAAACGAGCTGCCAATCGCCGGTGAGCCAGCGAACATCGTCGCGCTGGTCGAAGAATA |
| | CATGGACTGGCTGCACCAGTCCCCTGTCCCGAAGCTGCTGTTCTGGGGCACCCCAGGCGTTCTGATCC |
| | CACCGGCCGAAGCCGCTCGCCTGGCCAAAAGCCTGCCTAACTGCAAGGCTGTGGACATCGGCCCGGG |
| | TCTGAATCTGCTGCAAGAAGACAACCCGGACCTGATCGGCAGCGAGATCGCGCGCTGGCTGTCTACTCT |
| | GGAGATTTCCGGCTCCGGATCCGACTACAAGGACGATGACGACAAGTGA (SEQ ID NO: 23) |
| M HALO | ATGGGCAGTTCTAAGAGCAAGCCCAAGGATCCCAGCCAGCGGCGGAACAACAACAATGGACCTGTGGC |
| | CACTGAATTCGGCGGCTCCGAAATCGGTACTGGCTTTCCATTCGACCCCCATTATGTGGAAGTCCTGGG |
| | CGAGCGCATGCACTACGTCGATGTTGGTCCGCGCGATGGCACCCCTGTGCTGTTCCTGCACGGTAACC |
| | CGACCTCCTCCTACGTGTGGCGCAACATCATCCCGCATGTTGCACCGACCCATCGCTGCATTGCTCCAG |
| | ACCTGATCGGTATGGGCAAATCCGACAAACCAGACCTGGGTTATTTCTTCGACGACCACGTCCGCTTCAT |
| | GGATGCCTTCATCGAAGCCCTGGGTCTGGAAGAGGTCGTCCTGGTCATTCACGACTGGGGCTCCGCTC |
| | TGGGTTTCCACTGGGCCAAGCGCAATCCAGAGCGCGTCAAAGGTATTGCATTTATGGAGTTCATCCGCC |
| | CTATCCCGACCTGGGACGAATGGCCAGAATTTGCCCGCGAGACCTTCCAGGCCTTCCGCACCACCGAC |
| | GTCGGCCGCAAGCTGATCATCGATCAGAACGTTTTTATCGAGGGTACGCTGCCGATGGGTGTCGTCCGC |
| | CCGCTGACTGAAGTCGAGATGGACCATTACCGCGAGCCGTTCCTGAATCCTGTTGACCGCGAGCCACTG |
| | TGGCGCTTCCCAAACGAGCTGCCAATCGCCGGTGAGCCAGCGAACATCGTCGCGCTGGTCGAAGAATA |
| | CATGGACTGGCTGCACCAGTCCCCTGTCCCGAAGCTGCTGTTCTGGGGCACCCCAGGCGTTCTGATCC |
| | CACCGGCCGAAGCCGCTCGCCTGGCCAAAAGCCTGCCTAACTGCAAGGCTGTGGACATCGGCCCGGG |
| | TCTGAATCTGCTGCAAGAAGACAACCCGGACCTGATCGGCAGCGAGATCGCGCGCTGGCTGTCTACTCT |
| | GGAGATTTCCGGCTCCGGATCCGACTACAAGGACGATGACGACAAGTGA (SEQ ID NO: 24) |
| PM HALO | ATGGGCTGCATCAAGAGCAAGCGGAAGGACAAGGACCTGGAACTGAAGCTGCGGATCCTGCAGAGCAC |
| | CGTGCCTAGAGCTAGAGATCCTCCAGTGGCCACAGAATTCGGCGGCTCCGAAATCGGTACTGGCTTTCC |
| | ATTCGACCCCCATTATGTGGAAGTCCTGGGCGAGCGCATGCACTACGTCGATGTTGGTCCGCGCGATG |
| | GCACCCCTGTGCTGTTCCTGCACGGTAACCCGACCTCCTCCTACGTGTGGCGCAACATCATCCCGCATG |
| | TTGCACCGACCCATCGCTGCATTGCTCCAGACCTGATCGGTATGGGCAAATCCGACAAACCAGACCTGG |
| | GTTATTTCTTCGACGACCACGTCCGCTTCATGGATGCCTTCATCGAAGCCCTGGGTCTGGAAGAGGTCG |
| | TCCTGGTCATTCACGACTGGGGCTCCGCTCTGGGTTTCCACTGGGCCAAGCGCAATCCAGAGCGCGTC |
| | AAAGGTATTGCATTTATGGAGTTCATCCGCCCTATCCCGACCTGGGACGAATGGCCAGAATTTGCCCGC |
| | GAGACCTTCCAGGCCTTCCGCACCACCGACGTCGGCCGCAAGCTGATCATCGATCAGAACGTTTTTATC |
| | GAGGGTACGCTGCCGATGGGTGTCGTCCGCCCGCTGACTGAAGTCGAGATGGACCATTACCGCGAGCC |
| | GTTCCTGAATCCTGTTGACCGCGAGCCACTGTGGCGCTTCCCAAACGAGCTGCCAATCGCCGGTGAGC |
| | CAGCGAACATCGTCGCGCTGGTCGAAGAATACATGGACTGGCTGCACCAGTCCCCTGTCCCGAAGCTG |
| | CTGTTCTGGGGCACCCCAGGCGTTCTGATCCCACCGGCCGAAGCCGCTCGCCTGGCCAAAAGCCTGCC |
| | TAACTGCAAGGCTGTGGACATCGGCCCGGGTCTGAATCTGCTGCAAGAAGACAACCCGGACCTGATCG |
| | GCAGCGAGATCGCGCGCTGGCTGTCTACTCTGGAGATTTCCGGCTCCGGATCCGACTACAAGGACGAT |
| | GACGACAAGTGA (SEQ ID NO: 25) |
| G HALO | ATGGACTACAAGGACGATGACGACAAGGAATTCGGCGGCTCCGAAATCGGTACTGGCTTTCCATTCGAC |
| | CCCCATTATGTGGAAGTCCTGGGCGAGCGCATGCACTACGTCGATGTTGGTCCGCGCGATGGCACCCC |
| | TGTGCTGTTCCTGCACGGTAACCCGACCTCCTCCTACGTGTGGCGCAACATCATCCCGCATGTTGCACC |
| | GACCCATCGCTGCATTGCTCCAGACCTGATCGGTATGGGCAAATCCGACAAACCAGACCTGGGTTATTT |
| | CTTCGACGACCACGTCCGCTTCATGGATGCCTTCATCGAAGCCCTGGGTCTGGAAGAGGTCGTCCTGGT |
| | CATTCACGACTGGGGCTCCGCTCTGGGTTTCCACTGGGCCAAGCGCAATCCAGAGCGCGTCAAAGGTA |
| | TTGCATTTATGGAGTTCATCCGCCCTATCCCGACCTGGGACGAATGGCCAGAATTTGCCCGCGAGACCT |
| | TCCAGGCCTTCCGCACCACCGACGTCGGCCGCAAGCTGATCATCGATCAGAACGTTTTTATCGAGGGTA |
| | CGCTGCCGATGGGTGTCGTCCGCCCGCTGACTGAAGTCGAGATGGACCATTACCGCGAGCCGTTCCTG |
| | AATCCTGTTGACCGCGAGCCACTGTGGCGCTTCCCAAACGAGCTGCCAATCGCCGGTGAGCCAGCGAA |
| | CATCGTCGCGCTGGTCGAAGAATACATGGACTGGCTGCACCAGTCCCCTGTCCCGAAGCTGCTGTTCTG |
| | GGGCACCCCAGGCGTTCTGATCCCACCGGCCGAAGCCGCTCGCCTGGCCAAAAGCCTGCCTAACTGCA |
| | AGGCTGTGGACATCGGCCCGGGTCTGAATCTGCTGCAAGAAGACAACCCGGACCTGATCGGCAGCGAG |
| | ATCGCGCGCTGGCTGTCTACTCTGGAGATTTCCGGCTCCGGATCCGGTGGTAGCTTTCGTAGTGATGGC |
| | AAAAAGAAAAAGAAGAAATCCAAGACCAAATGCCAGCTGCTGTGA (SEQ ID NO: 26) |
| PPF HALO | ATGGACTACAAGGACGATGACGACAAGGAATTCGGCGGCTCCGAAATCGGTACTGGCTTTCCATTCGAC |
| | CCCCATTATGTGGAAGTCCTGGGCGAGCGCATGCACTACGTCGATGTTGGTCCGCGCGATGGCACCCC |
| | TGTGCTGTTCCTGCACGGTAACCCGACCTCCTCCTACGTGTGGCGCAACATCATCCCGCATGTTGCACC |
| | GACCCATCGCTGCATTGCTCCAGACCTGATCGGTATGGGCAAATCCGACAAACCAGACCTGGGTTATTT |
| | CTTCGACGACCACGTCCGCTTCATGGATGCCTTCATCGAAGCCCTGGGTCTGGAAGAGGTCGTCCTGGT |
| | CATTCACGACTGGGGCTCCGCTCTGGGTTTCCACTGGGCCAAGCGCAATCCAGAGCGCGTCAAAGGTA |
| | TTGCATTTATGGAGTTCATCCGCCCTATCCCGACCTGGGACGAATGGCCAGAATTTGCCCGCGAGACCT |
| | TCCAGGCCTTCCGCACCACCGACGTCGGCCGCAAGCTGATCATCGATCAGAACGTTTTTATCGAGGGTA |
| | CGCTGCCGATGGGTGTCGTCCGCCCGCTGACTGAAGTCGAGATGGACCATTACCGCGAGCCGTTCCTG |
| | AATCCTGTTGACCGCGAGCCACTGTGGCGCTTCCCAAACGAGCTGCCAATCGCCGGTGAGCCAGCGAA |
| | CATCGTCGCGCTGGTCGAAGAATACATGGACTGGCTGCACCAGTCCCCTGTCCCGAAGCTGCTGTTCTG |
| | GGGCACCCCAGGCGTTCTGATCCCACCGGCCGAAGCCGCTCGCCTGGCCAAAAGCCTGCCTAACTGCA |
| | AGGCTGTGGACATCGGCCCGGGTCTGAATCTGCTGCAAGAAGACAACCCGGACCTGATCGGCAGCGAG |
| | ATCGCGCGCTGGCTGTCTACTCTGGAGATTTCCGGCTCCGGATCCGGTGGTAGCATGAAAAAGCATCGT |
| | TGTAAATGTTGCAGCATTATGTGA (SEQ ID NO: 27) |

TABLE 5
DNA sequences for transcription factor delivery studies.

| Construct | DNA sequence |
|---|---|
| Soluble COMET | TGGAATTCGGCGGCTCCCCTAAGAAAAAGCGCAAAGTCTCCGGAAGCCAGTACCTGCCTGACACCGA |
| p65 ZF6 | CGACCGGCACAGAATCGAGGAAAAGCGGAAGCGGACCTACGAGACATTCAAGAGCATCATGAAGAAG |
| | TCCCCATTCAGCGGCCCCACCGATCCTAGACCTCCACCTAGAAGAATCGCCGTGCCTAGCAGATCCA |
| | GCGCCTCTGTGCCTAAACCTGCTCCTCAGCCTTATCCTTTCACCAGCAGCCTGAGCACCATCAACTAC |
| | GACGAGTTCCCCACAATGGTGTTCCCCAGCGGACAGATCAGCCAGGCTTCTGCTCTTGCTCCAGCTC |
| | CTCCTCAGGTTCTGCCTCAAGCTCCTGCTCCGGCTCCAGCACCAGCTATGGTTTCTGCTTTGGCCCAG |
| | GCTCCTGCACCTGTGCCTGTTCTTGCTCCTGGACCACCTCAGGCTGTTGCTCCACCAGCTCCTAAACC |
| | TACACAGGCCGGCGAGGGAACACTGTCTGAAGCCCTGCTGCAACTCCAGTTCGACGACGAGGATCTG |
| | GGAGCACTGCTGGGCAATAGCACAGACCCTGCCGTGTTTACCGATCTGGCCAGCGTGGACAACAGCG |
| | AGTTTCAGCAGCTCCTGAACCAGGGCATCCCTGTGGCTCCTCACACCACAGAGCCCATGCTGATGGA |
| | ATACCCCGAGGCCATCACCAGACTGGTCACCGGCGCTCAAAGACCTCCAGATCCTGCACCAGCACCT |
| | CTTGGAGCACCTGGCCTGCCTAATGGACTGCTGAGCGGAGATGAGGACTTCAGCTCTATCGCCGACA |
| | TGGATTTCAGCGCCCTGCTCGgttccGGTGGTGGCGGGTCTGGAGGAGGAGGTAGTGGTGGAGGTGGC |
| | TCTggtacCGCTAGACCCGGCGAAAGACCTTTCCAGTGCCGGATCTGCATGAGGAACTTCAGCAAGGGC |
| | GAGAGACTCGTGCGGCACACCAGAACACACACAGGCGAGAAGCCCTTCCAGTGTAGAATCTGTATGC |
| | GCAACTTCAGCCGGATGGACAACCTGAGCACCCACCTGAGAACCCATACCGGGGAGAAGCCATTTCA |
| | ATGCCGCATCTGTATGAGAAATTTTTCCCGGAAGGACGCCCTGAACCGGCACCTGAAAACACACCTGA |
| | GAGGCAGCGGCTCCGGATCCGACTACAAGGACGATGACGACAAGTGA (SEQ ID NO: 28) |
| M COMET p65 | ATGGGCAGTTCTAAGAGCAAGCCCAAGGAcCCtAGCCAGCGGCGGAACAACAACAATGGACCTGTGG |
| ZF6 | CCACTGAATTCGGCGGCTCCCCTAAGAAAAAGCGCAAAGTCTCCGGAAGCCAGTACCTGCCTGACAC |
| | CGACGACCGGCACAGAATCGAGGAAAAGCGGAAGCGGACCTACGAGACATTCAAGAGCATCATGAAG |
| | AAGTCCCCATTCAGCGGCCCCACCGATCCTAGACCTCCACCTAGAAGAATCGCCGTGCCTAGCAGAT |
| | CCAGCGCCTCTGTGCCTAAACCTGCTCCTCAGCCTTATCCTTTCACCAGCAGCCTGAGCACCATCAAC |
| | TACGACGAGTTCCCCACAATGGTGTTCCCCAGCGGACAGATCAGCCAGGCTTCTGCTCTTGCTCCAG |
| | CTCCTCCTCAGGTTCTGCCTCAAGCTCCTGCTCCGGCTCCAGCACCAGCTATGGTTTCTGCTTTGGCC |
| | CAGGCTCCTGCACCTGTGCCTGTTCTTGCTCCTGGACCACCTCAGGCTGTTGCTCCACCAGCTCCTAA |
| | ACCTACACAGGCCGGCGAGGGAACACTGTCTGAAGCCCTGCTGCAACTCCAGTTCGACGACGAGGAT |
| | CTGGGAGCACTGCTGGGCAATAGCACAGACCCTGCCGTGTTTACCGATCTGGCCAGCGTGGACAACA |
| | GCGAGTTTCAGCAGCTCCTGAACCAGGGCATCCCTGTGGCTCCTCACACCACAGAGCCCATGCTGAT |
| | GGAATACCCCGAGGCCATCACCAGACTGGTCACCGGCGCTCAAAGACCTCCAGATCCTGCACCAGCA |
| | CCTCTTGGAGCACCTGGCCTGCCTAATGGACTGCTGAGCGGAGATGAGGACTTCAGCTCTATCGCCG |
| | ACATGGATTTCAGCGCCCTGCTCGgttccGGTGGTGGCGGGTCTGGAGGAGGAGGTAGTGGTGGAGGT |
| | GGCTCTggtacCGCTAGACCCGGCGAAAGACCTTTCCAGTGCCGGATCTGCATGAGGAACTTCAGCAAG |
| | GGCGAGAGACTCGTGCGGCACACCAGAACACACACAGGCGAGAAGCCCTTCCAGTGTAGAATCTGTA |
| | TGCGCAACTTCAGCCGGATGGACAACCTGAGCACCCACCTGAGAACCCATACCGGGGAGAAGCCATT |
| | TCAATGCCGCATCTGTATGAGAAATTTTTCCCGGAAGGACGCCCTGAACCGGCACCTGAAAACACACC |
| | TGAGAGGCAGCGGCTCCGGATCCGACTACAAGGACGATGACGACAAGTGA (SEQ ID NO: 29) |
| PM COMET p65 | ATGGGCTGCATCAAGAGCAAGCGGAAGGACAAGGACCTGGAACTGAAGCTGagaATCCTGCAGAGCA |
| ZF6 | CCGTGCCTAGAGCTAGAGATCCTCCAGTGGCCACAGAATTCGGCGGCTCCCCTAAGAAAAAGCGCAA |
| | AGTCTCCGGAAGCCAGTACCTGCCTGACACCGACGACCGGCACAGAATCGAGGAAAAGCGGAAGCG |
| | GACCTACGAGACATTCAAGAGCATCATGAAGAAGTCCCCATTCAGCGGCCCCACCGATCCTAGACCTC |
| | CACCTAGAAGAATCGCCGTGCCTAGCAGATCCAGCGCCTCTGTGCCTAAACCTGCTCCTCAGCCTTAT |
| | CCTTTCACCAGCAGCCTGAGCACCATCAACTACGACGAGTTCCCCACAATGGTGTTCCCCAGCGGAC |
| | AGATCAGCCAGGCTTCTGCTCTTGCTCCAGCTCCTCCTCAGGTTCTGCCTCAAGCTCCTGCTCCGGCT |
| | CCAGCACCAGCTATGGTTTCTGCTTTGGCCCAGGCTCCTGCACCTGTGCCTGTTCTTGCTCCTGGACC |
| | ACCTCAGGCTGTTGCTCCACCAGCTCCTAAACCTACACAGGCCGGCGAGGGAACACTGTCTGAAGCC |
| | CTGCTGCAACTCCAGTTCGACGACGAGGATCTGGGAGCACTGCTGGGCAATAGCACAGACCCTGCCG |
| | TGTTTACCGATCTGGCCAGCGTGGACAACAGCGAGTTTCAGCAGCTCCTGAACCAGGGCATCCCTGT |
| | GGCTCCTCACACCACAGAGCCCATGCTGATGGAATACCCCGAGGCCATCACCAGACTGGTCACCGGC |
| | GCTCAAAGACCTCCAGATCCTGCACCAGCACCTCTTGGAGCACCTGGCCTGCCTAATGGACTGCTGA |
| | GCGGAGATGAGGACTTCAGCTCTATCGCCGACATGGATTTCAGCGCCCTGCTCGgttccGGTGGTGGCG |
| | GGTCTGGAGGAGGAGGTAGTGGTGGAGGTGGCTCTggtacCGCTAGACCCGGCGAAAGACCTTTCCAG |
| | TGCCGGATCTGCATGAGGAACTTCAGCAAGGGCGAGAGACTCGTGCGGCACACCAGAACACACACAG |
| | GCGAGAAGCCCTTCCAGTGTAGAATCTGTATGCGCAACTTCAGCCGGATGGACAACCTGAGCACCCA |
| | CCTGAGAACCCATACCGGGGAGAAGCCATTTCAATGCCGCATCTGTATGAGAAATTTTTCCCGGAAGG |
| | ACGCCCTGAACCGGCACCTGAAAACACACCTGAGAGGCAGCGGCTCCGGATCCGACTACAAGGACG |
| | ATGACGACAAGTGA (SEQ ID NO: 30) |
| G COMET p65 | ATGGACTACAAGGACGATGACGACAAGGAATTCGGCGGCTCCCCTAAGAAAAAGCGCAAAGTCTCCG |
| ZF6 | GAAGCCAGTACCTGCCTGACACCGACGACCGGCACAGAATCGAGGAAAAGCGGAAGCGGACCTACG |
| | AGACATTCAAGAGCATCATGAAGAAGTCCCCATTCAGCGGCCCCACCGATCCTAGACCTCCACCTAGA |
| | AGAATCGCCGTGCCTAGCAGATCCAGCGCCTCTGTGCCTAAACCTGCTCCTCAGCCTTATCCTTTCAC |
| | CAGCAGCCTGAGCACCATCAACTACGACGAGTTCCCCACAATGGTGTTCCCCAGCGGACAGATCAGC |
| | CAGGCTTCTGCTCTTGCTCCAGCTCCTCCTCAGGTTCTGCCTCAAGCTCCTGCTCCGGCTCCAGCACC |
| | AGCTATGGTTTCTGCTTTGGCCCAGGCTCCTGCACCTGTGCCTGTTCTTGCTCCTGGACCACCTCAGG |
| | CTGTTGCTCCACCAGCTCCTAAACCTACACAGGCCGGCGAGGGAACACTGTCTGAAGCCCTGCTGCA |
| | ACTCCAGTTCGACGACGAGGATCTGGGAGCACTGCTGGGCAATAGCACAGACCCTGCCGTGTTTACC |
| | GATCTGGCCAGCGTGGACAACAGCGAGTTTCAGCAGCTCCTGAACCAGGGCATCCCTGTGGCTCCTC |
| | ACACCACAGAGCCCATGCTGATGGAATACCCCGAGGCCATCACCAGACTGGTCACCGGCGCTCAAAG |
| | ACCTCCAGATCCTGCACCAGCACCTCTTGGAGCACCTGGCCTGCCTAATGGACTGCTGAGCGGAGAT |
| | GAGGACTTCAGCTCTATCGCCGACATGGATTTCAGCGCCCTGCTCGgttccGGTGGTGGCGGGTCTGGA |
| | GGAGGAGGTAGTGGTGGAGGTGGCTCTggtacCGCTAGACCCGGCGAAAGACCTTTCCAGTGCCGGAT |
| | CTGCATGAGGAACTTCAGCAAGGGCGAGAGACTCGTGCGGCACACCAGAACACACACAGGCGAGAA |
| | GCCCTTCCAGTGTAGAATCTGTATGCGCAACTTCAGCCGGATGGACAACCTGAGCACCCACCTGAGA |
| | ACCCATACCGGGGAGAAGCCATTTCAATGCCGCATCTGTATGAGAAATTTTTCCCGGAAGGACGCCCT |
| | GAACCGGCACCTGAAAACACACCTGAGAGGCAGCGGCTCCGGATCCGGTGGTAGCTTTCGTAGTGAT |
| | GGCAAAAAGAAAAAGAAGAAATCCAAGACCAAATGCCAGCTGCTGTGA (SEQ ID NO: 31) |
| PPF COMET p65 | ATGGACTACAAGGACGATGACGACAAGGAATTCGGCGGCTCCCCTAAGAAAAAGCGCAAAGTCTCCG |
| ZF6 | GAAGCCAGTACCTGCCTGACACCGACGACCGGCACAGAATCGAGGAAAAGCGGAAGCGGACCTACG |
| | AGACATTCAAGAGCATCATGAAGAAGTCCCCATTCAGCGGCCCCACCGATCCTAGACCTCCACCTAGA |
| | AGAATCGCCGTGCCTAGCAGATCCAGCGCCTCTGTGCCTAAACCTGCTCCTCAGCCTTATCCTTTCAC |
| | CAGCAGCCTGAGCACCATCAACTACGACGAGTTCCCCACAATGGTGTTCCCCAGCGGACAGATCAGC |
| | CAGGCTTCTGCTCTTGCTCCAGCTCCTCCTCAGGTTCTGCCTCAAGCTCCTGCTCCGGCTCCAGCACC |
| | AGCTATGGTTTCTGCTTTGGCCCAGGCTCCTGCACCTGTGCCTGTTCTTGCTCCTGGACCACCTCAGG |
| | CTGTTGCTCCACCAGCTCCTAAACCTACACAGGCCGGCGAGGGAACACTGTCTGAAGCCCTGCTGCA |
| | ACTCCAGTTCGACGACGAGGATCTGGGAGCACTGCTGGGCAATAGCACAGACCCTGCCGTGTTTACC |
| | GATCTGGCCAGCGTGGACAACAGCGAGTTTCAGCAGCTCCTGAACCAGGGCATCCCTGTGGCTCCTC |
| | ACACCACAGAGCCCATGCTGATGGAATACCCCGAGGCCATCACCAGACTGGTCACCGGCGCTCAAAG |
| | ACCTCCAGATCCTGCACCAGCACCTCTTGGAGCACCTGGCCTGCCTAATGGACTGCTGAGCGGAGAT |
| | GAGGACTTCAGCTCTATCGCCGACATGGATTTCAGCGCCCTGCTCGgttccGGTGGTGGCGGGTCTGGA |
| | GGAGGAGGTAGTGGTGGAGGTGGCTCTggtacCGCTAGACCCGGCGAAAGACCTTTCCAGTGCCGGAT |
| | CTGCATGAGGAACTTCAGCAAGGGCGAGAGACTCGTGCGGCACACCAGAACACACACAGGCGAGAA |
| | GCCCTTCCAGTGTAGAATCTGTATGCGCAACTTCAGCCGGATGGACAACCTGAGCACCCACCTGAGA |
| | ACCCATACCGGGGAGAAGCCATTTCAATGCCGCATCTGTATGAGAAATTTTTCCCGGAAGGACGCCCT |
| | GAACCGGCACCTGAAAACACACCTGAGAGGCAGCGGCTCCGGATCCGGTGGTAGCATGAAAAAGCAT |
| | CGTTGTAAATGTTGCAGCATTATGTGA (SEQ ID NO: 32) |

Golden gate assembly of SynTF reporter integration vector: The SynTF (specifically, ZF658) reporter integration vector was assembled as a landing pad-based integration vector through a BbsI-mediated Golden Gate reaction. Each 20 μL reaction comprised 2 μL 10×T4 ligase buffer, 2 μL 10×BSA (1 mg/mL stock), 0.8 μL BbsI-HF restriction enzyme (NEB #R3539L), 0.8 μL T4 DNA Ligase (400 U/μL stock), 20 fmol integration vector backbone and 40 fmol of each transcription unit and linker plasmid to be inserted. The backbone used here was pPD1178, which contains a promoter-less expression unit of the puromycin resistance gene and miRFP720 connected via P2A; both genes are expressed upon successful integration in the landing pad downstream of the landing pad's CAG promoter. See, Duportet, X. et al. Nucleic Acids Res 42, 13440 (2014). The reaction also included six transcription units of the SynTF (ZF6) promoter with 12 compact binding sites upstream of a YB_TATA minimal promoter and a DsRedExpress2 reporter gene (pHIE324-329), a seventh transcription unit containing a constitutive hEFla promoter and a blasticidin resistance gene (pHIE280), and a linker plasmid to close the assembly (pPD1157). See, Hansen, J. et al. Proc Natl Acad Sci USA 111, 15705-15710 (2014); Ede, C. et al. ACS Synth. Biol 5, (2016). Transcription unit vectors for each reporter copy and the linker vector were previously described. See, Donahue, P. S. et al. Nature Communications 2020 11:1 11, 1-19 (2020). The reaction was incubated at 37° C. for 15 min, then subjected to 55 iterations of thermocycling (37° C. for 5 min, 16° C. for 3 min, repeat), followed by 37° C. for 15 min, 50° C. for 5 min, 80° C. for 10 min to terminate the reactions; then the mixture was cooled to room temperature (~22° C.) prior to immediate transformation into NEB Stable chemically competent bacteria (NEB #C3040H).

Cell culture: The HEK293FT cell line was purchased from Thermo Fisher/Life Technologies. The HEK293LP cell line was kindly gifted. Cells were cultured in Dulbecco's Modified Eagle Medium (DMEM) (Gibco 31600-091) with 10% FBS (Gibco 16140-071), 6 mM L-glutamine (2 mM from Gibco 31600-091 and 4 mM from additional Gibco 25030-081), penicillin (100 U/μL), and streptomycin (100 μg/mL) (Gibco 15140122), in a 37° C. incubator with 5% CO2. Prior to flow cytometry and for certain microscopy studies, cells were cultured temporarily in phenol red-free DMEM from Sigma (D2902). This base medium was supplemented with 4 mg/L pyridoxine-HCl (Sigma P6280), 16 mg/L sodium phosphate (Sigma S5011), 3.7 g/L sodium bicarbonate, 3.5 g/L glucose, 100 U/mL penicillin, 100 μg/mL streptomycin, 4 mM L-glutamine, and 10% FBS.

Laurdan spectroscopy and liposome formation: Liquid disordered membranes were composed of 70 mol % 1-palmitoyl-2-oleoyl-glycero-3-phosphocholine (POPC) and 30 mol % cholesterol, while liquid ordered membranes were composed of 70 mol % 1,2-dipalmitoyl-sn-glycero-3-phosphocholine (DPPC) and 30 mol % cholesterol. Lipids were purchased from Avanti. Liposomes were prepared via the thin film hydration method. Lipids dissolved in chloroform were dried under nitrogen gas in a glass vial and placed in a vacuum oven for at least 2 h. Films were rehydrated with PBS at 60° C. for at least 2 h before being vortexed and extruded (21 passes) through a 100 nm polycarbonate filter (Avanti Mini Extruder).

C-Laurdan was used to measure the membrane fluidity of cell, EV, and liposome membranes. 100 μM of lipid was used for Laurdan measurements of liposomes. Approximately, 1×10⁹ microvesicles and exosomes, and 1×10⁶ cells were used in each measurement. For bulk measurements, 0.4 μM C-Laurdan (Tocris, 7273) was added to the sample of interest and incubated in the dark at room temperature for 30 min. C-Laurdan spectra was then read (ex. 350 nm) using a Molecular Devices Spectra Max i3 plate reader. Membrane hydration was analyzed by calculating Laurdan generalized fluorescence polarization (GP) through the following formula:

$$GP = {I}_{439} - {I}_{483} / {I}_{439} + {I}_{483}$$

Where I439 and I483 are the fluorescence intensities of the sample at 439 and 483 nm, respectively, when excited at 350 nm.

Transient transfection: Transient transfection of HEK293FT cells was achieved using the calcium phosphate method, described previously. See, Donahue, P. S. et al. Nature Communications 2020 11:1 11, 1-19 (2020). Briefly, cells were plated in DMEM and allowed to adhere to the plate for 5-8 h. Plasmids encoding the protein of interest were diluted in a water and 0.3 M CaCl₂) solution, added to an equal volume of 2× HEPES Buffered Saline (280 mM NaCl, 50 mM HEPES, 1.5 mM Na2HPO4), mixed 4 times, and incubated for 2-4 min. The DNA solution was then vigorously pipetted and added dropwise to plated cells. Plating density and DNA amounts added for each experiment and plate format can be found in the sections below. The next morning, medium was aspirated and replaced with fresh DMEM. Cells were cultured for at least an additional 24 h before performing downstream experiments.

Generation of the HEK293FT SynTF (ZF6) reporter cell line (HIE156): From exponentially growing HEK293LP cells (see Duportet, X. et al. Nucleic Acids Res 42, 13440 (2014)), 0.5×10⁵ cells were plated per well (0.5 mL medium) in 24-well format, and cells were cultured for 24 h to allow cells to attach and spread. Bxb1 recombinase was co-transfected with the integration vector by lipofection with Lipofectamine LTX with PLUS Reagent (ThermoFisher 15338100). 300 ng of BxB1 expression vector was mixed with 300 ng of integration vector, 0.5 μL of PLUS reagent, and enough OptiMEM (ThermoFisher/Gibco 31985062) to bring the mix volume up to 25 μL. In a separate tube, 1.9 μL of LTX reagent was mixed with 23.1 μL of OptiMEM. The DNA/PLUS reagent mix was added to the LTX mix, pipetted up and down four times, and then incubated at room temperature for 5 min. 50 μL of this transfection mix was added dropwise to each well of cells and mixed by gentle swirling. Cells were cultured until the well was ready to split (typically 3 d), without any media changes.

To begin selection of cells that successfully integrated the SynTF reporter integration vector, cells were harvested from the 24-well plate when confluent by trypsinizing and transferring to a single well of a 6-well plate in 2 mL of medium supplemented with 1 μg/mL puromycin (Invivogen ant-pr). Cells were trypsinized daily (typically 3 d) until cell death was no longer evident. Cells were cultured in medium supplemented with puromycin until the 6-well was confluent and cells were exponentially growing. Cells were then selected with 6 μg/mL blasticidin (Alfa Aesar/ThermoFisher J61883) for 7 d. Cells were cultured in both puromycin and blasticidin to maintain selective pressure until flow sorting.

To sort, cells were harvested by trypsinizing, resuspended at approximately 10⁷ cells per mL in pre-sort medium (DMEM with 10% FBS, 25 mM HEPES (Sigma H3375), and 100 μg/mL gentamycin (Amresco 0304)), and held on ice until sorting was performed. Cells were sorted using a BD FACS Aria 4-laser Special Order Research Product (Robert H. Lurie Cancer Center Flow Cytometry Core). The sorting strategy was as follows: single cells were first gated to exclude all EYFP positive cells (as EYFP positive cells still have an intact landing pad locus, suggesting a mis-integration event occurred) and to include only miRFP720+ cells. EYFP expression was measured using the FITC channel (488 nm excitation laser, 505 LP and 525/30 emission filters) and miRFP720 expression was measured using a modified APC-Cy7 channel (640 nm excitation laser, 690 LP and 730/45 emission filters). Then a gate was drawn on miRFP720 expression to capture the 88th to 98th percentile of miRFP720-expressing cells (the top 2% were excluded to exclude cells suspected to possess two or more integrated copies of the cargo vector). 50,000 cells were collected in post-sort medium (DMEM with 20% FBS, 25 mM HEPES, and 100 μg/mL gentamycin), and cells were held on ice until they could be centrifuged at 150 g for 5 min, resuspended in 0.5 mL complete medium supplemented with 100 μg/mL gentamycin, and plated in one well of a 24-well plate. Cells were maintained in gentamycin for 7 d after sorting during expansion before banking. Cells were thawed for use in experiments in this study.

Protein trafficking analyzed via live cell imaging: Protein localization to the plasma membrane and endoplasmic reticulum was analyzed in live cells using confocal microscopy. To facilitate strong cell attachment, well plates were coated with poly-L-lysine (Sigma P6282, resuspended at 0.1 mg/mL in sterile water) by incubating wells with poly-L-lysine for at least 5 min at room temperature before aspirating excess solution and allowing the plates to dry. Cells were plated into treated wells and transfected with 20 ng of DNA encoding transmembrane proteins and 60 ng of DNA encoding for peripheral membrane proteins. 48 hours after transfection, cells were imaged. All proteins were fused to either RFP or HaloTag. For proteins fused to HaloTag, proteins were tagged with TMR ligand for visualization. TMR Ligand (Promega) was diluted 1:200 in PBS and used as a 5× stock. One-fifth of the existing volume of cell medium was replaced with the TMR HaloTag solution (5 μM TMR final concentration) and incubated for 15 min in a 37° C. and 5% CO₂ cell culture incubator. The ligand containing medium was then gently washed with fresh medium and replaced with 500 μL of fresh medium containing 5 μM ER Tracker Blue-White DPX (Thermo Fisher) and 1:1000 Cell-Mask Deep Red (Thermo Fisher). Cells were then incubated at 37° C. and 5% CO₂ in a cell culture incubator for 30 min. Medium was then replaced with phenol red-free DMEM. Once labeled, cells were imaged using a Nikon confocal microscope. A 561 nm laser was used to excite the HaloTag or mRFP1 labeled proteins, a 405 nm laser was used to excite the endoplasmic reticulum dye (ER Tracker Blue-White DPX), and a 640 nm laser was used to excite the plasma membrane dye (Cell-Mask Deep Red). To observe the entire cell, Z-stacks were captured and converted into maximum projection image prior to analysis. Correlation of protein localization with the plasma membrane and endoplasmic reticulum was calculated using the Nikon NIS Elements software for whole cells.

Measuring protein raft localization in GPMVs: Protein association with lipid rafts was measured using GPMVs. Cells were plated into 6-well plates and transfected with 80 ng of DNA encoding transmembrane proteins and 240 ng of DNA encoding for peripheral membrane proteins. 48 h after transfection, GPMVs were produced. All proteins were fused to either RFP or HaloTag. For proteins fused to HaloTag, proteins were tagged with TMR ligand for visualization. TMR Ligand (Promega) was diluted 1:200 in PBS and used as a 10× stock. One-tenth of the existing volume of cell medium was replaced with the TMR HaloTag solution (2.5 μM TMR final concentration) and incubated for 15 min in a 37° C. and 5% CO₂ cell culture incubator. Cells were then washed with GPMV buffer (5 mM HEPES, 75 mM NaCl, 1 mM CaCl2, pH 7.4) and then incubated for 1 h in a 37° C. and 5% CO₂ cell culture incubator in GPMV vesiculation buffer (GPMV buffer with 0.08% PFA and 2 mM DTT). GPMVs were then carefully collected, making sure to not disturb the cells, and allowed to sink in a fresh tube. Prior to imaging, GPMVs were labeled with 5 μg/mL of Fast DiO. Samples were then placed onto a glass slide and cooled to 10° C. on the microscope stage using a Linkam PE100 Peltier Stage. Once the sample temperature reached 10° C., GPMVs were imaged using a Nikon confocal microscope. A 561 nm laser was used to excite the protein label, and a 488 nm laser was used to excite the membrane dye. Line scans were performed to measure the protein fluorescence in raft (IRaft) and nonraft (INon Raft) regions, as marked by DiO. Protein partitioning (L_{O} enrichment) in lipid rafts was calculated using the Nikon NIS Elements Software.

L_{O} Enrichment was calculated using the following equation:

$${L}_{o} Enrichment = \frac{{I}_{Raft} - {I}_{NonRaft}}{{I}_{Raft} + {I}_{NonRaft}}$$

Where I_{Raft} represents the protein fluorescence intensity in the raft phase, and I_{Non Raft} represents the protein fluorescence intensity in the nonraft phase.

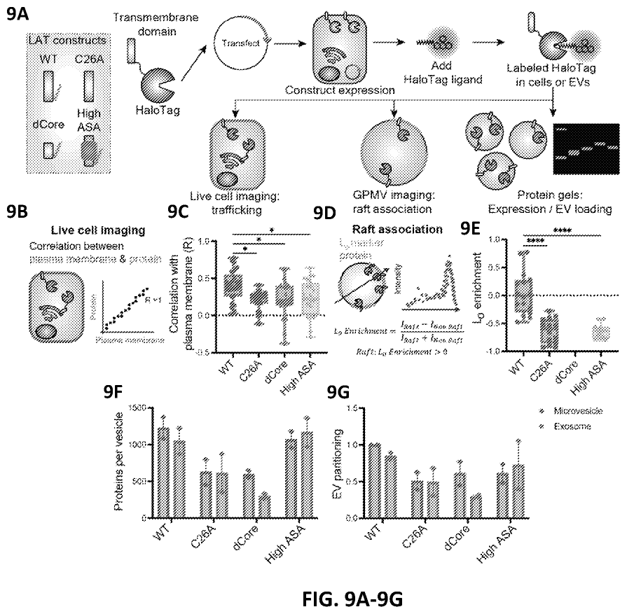

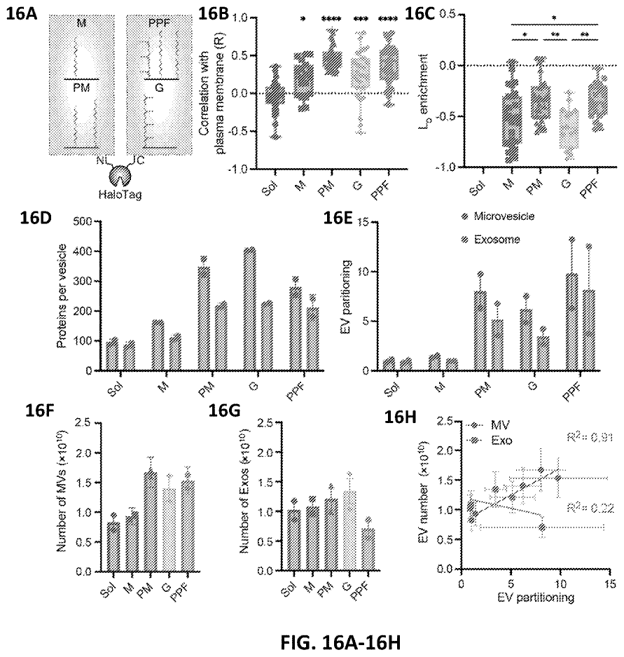

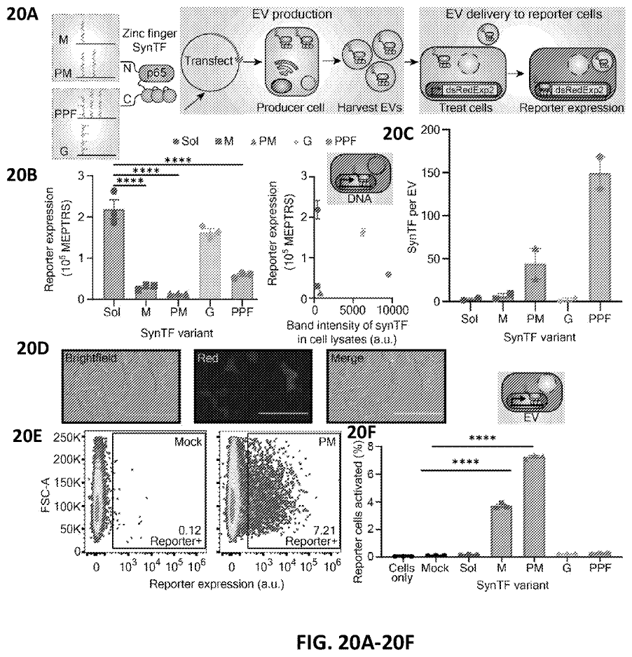

EV production and isolation: 5×10⁶ HEK293FTs were plated in 10-cm tissue culture treated plates in 8-10 mL DMEM. 5-8 h later, cells were transfected as described above (“Transient transfection”). Total DNA amount was between 5-20 ug per plate and kept consistent for all conditions in a given experiment. The typical amount of plasmid encoding the protein of interest was 2-5 μg per plate. The remainder of plasmid was empty vector (pcDNA) and for EV transfections with HaloTag- or SynTF-containing constructs (FIG. 9F, 9G, FIG. 16D, 16E, FIG. 20C) a blue transfection marker (eBFP2). The next morning after transfection, medium was replaced with DMEM supplemented with 10% of EV-depleted FBS (Gibco A2720801 or Omega Scientific FB40/50). Supernatant was harvested for EV isolation approximately 30-34 h later and clarified by sequential centrifugation at 300 g for 10 min and 2000 g for 20 min. Microvesicles were pelleted by a Beckman Coulter Avanti J-26XP centrifuge with a J-LITE JLA 16.25 rotor at 15,000 g for 30 min. Exosomes were pelleted by centrifuging the supernatant at 120,416 g for 135 min in polypropylene ultracentrifuge tubes in a Beckman Coulter Optima L-80 XP model and a SW 41 Ti rotor. EVs were resuspended in the conditioned cell medium remaining in their tubes via gentle pipetting. Each centrifugation step was performed at 4° C., and samples were stored on ice for short-term use or at −80° C. for long-term storage.

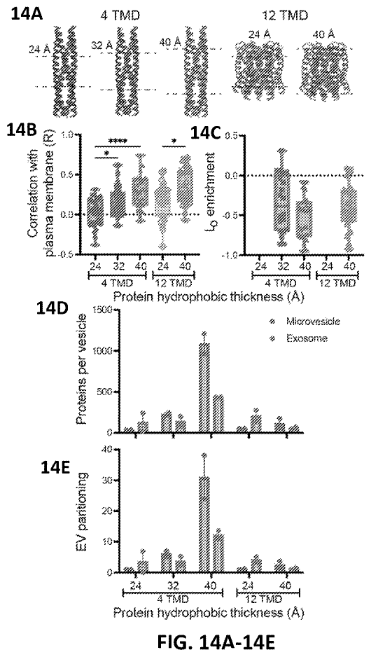

Nanoparticle tracking analysis and production measurements: EV concentration and size were measured via a NanoSight NS300 (Malvern) instrument with a 642 nm laser on software v3.4. Evs were diluted in PBS to 2-10×10⁸ particles/mL, infused at setting 30, imaged with camera level 14, and analyzed at detection threshold 7. Data reported are averaged from the analysis of three 30 s videos per sample. For data presented in FIG. 9A-9G, FIG. 14A-14E, FIG. 16A-16H, the number of Evs produced for a given experiment were collected from two 10 cm plates of transfected HEK293FTs.

Cell lysate isolation and BCA: Cell lysates were harvested from HEK293FTs using radioimmunoprecipitation assay buffer (RIPA); samples were collected at the same time as EV supernatants. Briefly, the medium was removed, the cells were washed 2× in ice cold PBS and then lysed with ice cold RIPA (150 mM NaCl, 50 mM Tris-HCl pH 8.0, 1% Triton X-100, 0.5% sodium deoxycholate, 0.1% sodium dodecyl sulfate, and protease inhibitor (Pierce #A32953)). Lysates were transferred to cold microcentrifuge tubes and incubated for at least 30 min on ice. Samples were clarified by centrifugation at 14,000 g×20 min at 4° C., and the supernatant was transferred to fresh tubes. Protein concentration was determined via bicinchoninic acid (BCA) assay (Pierce #23225), and samples were kept on ice for short-term storage or frozen at −80° C. for long-term storage.

Quantification of protein loading into EVs: Protein loading into EVs was quantified either via fluorescent PAGE gel or western blot. For protein constructs fused to HaloTag, cell lysates and EVs were incubated with 1 μL of 1:100 TMR ligand at room temperature for 15 min. Laemmli buffer was then added and samples were heated to 70° C. for 3 min. Approximately 5×10⁸ vesicles and purified HaloTag standard curve were loaded and run on a 12% Mini-PROTEAN TGX Precast Protein Gel (Bio-Rad) at 150 V for 90 min or 50V for 10 min followed by 100V for approximately 70 min. 5 μg of cell lysate for each construct was run on a separate gel. Once complete, gels were washed in Milli-Q water and imaged using an Azure Sapphire Imager. Gels were analyzed using ImageJ (Fiji 3).

For protein constructs without HaloTag, PAGE gels were run in the same manner, except for the HaloTag ligand incubation step. Additionally, purified FLAG tagged protein (recombinant p53 protein, R&D Systems) was run with vesicles to quantify loading. Wet transfer was performed onto a PVDF membrane (Bio-Rad) for 45 min at 100 V. Membranes were then blocked for an hour at room temperature in 5% milk in TBST (pH 7.6:50 mM Tris, 150 mM NaCl, HCl to pH 7.6, 0.1% Tween 20) and incubated for 1 h at room temperature or overnight at 4° C. with primary solution (anti-FLAG (Sigma F1804), diluted 1:1000 in 5% milk in TBST). Primary antibody solution was decanted, and the membrane was washed three times for 5 min in TBST and then incubated in secondary solution at room temperature for 1 h (HRP-anti-Mouse (CST 7076) diluted 1:3000 in 5% milk in TBST). Membranes were then washed in TBST and incubated with Clarity Western ECL Substrate (Bio-Rad) for 5 min. Blots were imaged using an Azure Biosystems c280 imager, and band intensities were quantified with ImageJ. Exceptions to the above protocol are listed in Table 6.

TABLE 6
Western blot antibodies used in this study and
associated sample preparation considerations

| | | Denature | | Reducing/Non- | |
|---|---|---|---|---|---|
| Antibody | | temperature/ | Antibody | reducing | Animal of |
| target | Supplier (#) | time | dilution | Laemmli | origin |
| FLAG tag | Sigma (F1804) | 70° C./10 min | 1:1000 | Reducing | Mouse |
| CD9 | Santa Cruz | 95° C./10 min | 1:500 | Reducing | Mouse |
| | (sc-13118) | | | | |
| CD81 | Santa Cruz | 95° C./10 min | 1:500 | Non-reducing | Mouse |
| | (sc-23962) | | | | |
| Alix | Abcam | 95° C./10 min | 1:500 | Reducing | Mouse |
| | (Ab117600) | | | | |
| Calnexin | Abcam | 70° C./10 min | 1:1000 | Reducing | Rabbit |
| | (Ab22595) | | | | |
| Rabbit | Invitrogen | Not applicable | 1:3000 | Not applicable | Goat |
| | (32460) | | | | |
| Mouse | Cell Signaling | Not applicable | 1:3000 | Not applicable | Horse |
| | Technology | | | | |
| | (7076) | | | | |

EV-mediated SynTF delivery experiments: To generate SynTF-containing EVs, HEK293FTs were transfected as described above (“EV production and isolation”) with plasmids encoding the following: SynTF variant (~5 μg), vesicular stomatitis virus G (VSV-G) viral fusion protein (3 μg), blue color control (eBFP2) (0.3-0.5 μg), and an empty-pcDNA3.1 backbone (to bring the total DNA transfected up to 10 μg per plate). Because each SynTF variant is slightly different in plasmid size, cells were transfected such that each plate received equal copy numbers of plasmids, generally around 5 μg. The Mock EV conditions were transfected as per above but with the DNA mass of the SynTF variant replaced with additional empty pcDNA3.1 backbone. EVs were harvested as described above, and the MV and exosome fractions were combined. EVs were diluted in fresh, complete DMEM and added to a tissue-cultured treated 48-well plate in 100 μL total volume per well. COMET reporter cells were harvested by washing once in PBS, a short incubation in trypsin (<1 min), and quenching with DMEM. Cells were counted and diluted in DMEM to 2×10⁵ cells per mL; 100 μL of this mixture was added to wells containing 100 μL EVs such that final cell count per well was 2×10⁴. Cell only conditions received 100 μL DMEM instead of EVs. Within each experiment, equal numbers of EVs as determined by NTA were added to each well, typical doses were between 3-4×10¹⁰ EVs per well. Plates were swirled to encourage mixing, and were returned to the incubator to culture for 2 d. In parallel, HEK293FTs were transfected with appropriate color compensation controls for flow cytometry as described above (“Transient transfection”). After 2 d, EV-treated cells were imaged on a Keyence BZ-x800 microscope. BZ Series Application software v01.01.00.17 was used, and cells were imaged with either a PlanApo 4× (NA 0.2), PlanApo 10× (0.45), or PlanFluor 20× (NA 0.45) objective. Fluorescence images were captured using a dsRed filter cube (Chroma 49005-UF1). Cells were then harvested for flow cytometry. Briefly, cells were harvested with trypsin, quenched with phenol-red free DMEM, and diluted in at least five volumes of fluorescence-activated cell sorting (FACS) buffer (PBS pH 7.4, 2 mM EDTA, 0.05% bovine serum albumin). Cells were pelleted at 150 g for 5 min at 4° C., the supernatant was decanted, and the samples were stored at 4° C. until flow cytometry analysis.

Transfected SynTF delivery experiments: In a tissue culture-treated 24-well plate, 1.5×10⁵ HEK293FTs were plated in 0.5 mL DMEM and allowed to adhere for 5-8 h. Cells were transfected as described above (“Transient transfection”) with equal copy numbers of each SynTF (approximately 50 ng), a blue color control, and empty backbone pcDNA plasmid such that each well received 500 ng total DNA. The media was changed the next morning, and cells were harvested for flow cytometry the following morning (approximately 2 d after transfection). Cells were washed in PBS, harvested with trypsin, quenched with phenol red-free DMEM, and diluted in at least five volumes of FACS buffer. Cells were pelleted at 150 g for 5 min at 4° C., the supernatant was decanted, and the samples were stored at 4° C. until flow cytometry analysis.

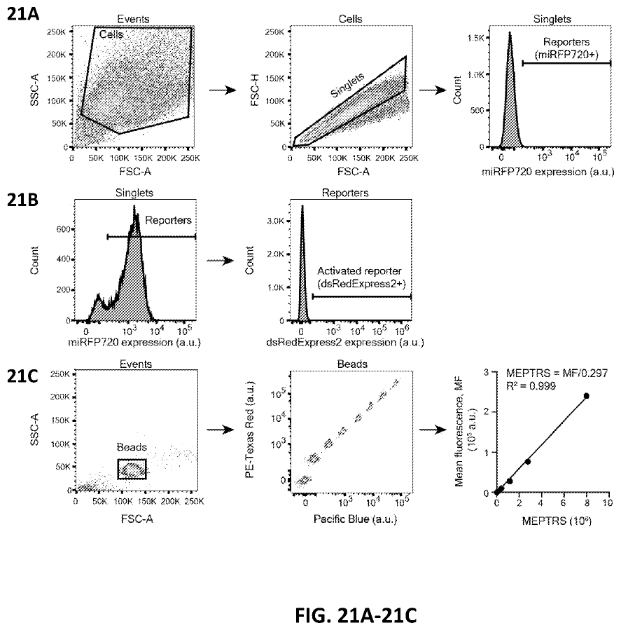

Flow Cytometry: Flow cytometry was performed using a BD LSR Fortessa Special Order Research Product (Robert H. Lurie Cancer Center Flow Cytometry Core). Lasers and filter sets used are in Table 7. Samples were analyzed using FlowJo V10.8.1 software. For experiments where transcription factor plasmids were directly transfected into reporter cells, approximately 3,000-6,000 single cells were analyzed. For all other EV-mediated transcription factor delivery experiments, approximately 30,000 to 60,000 single cells were analyzed. As shown in FIG. 21A-21C, HEK293FT cells were first gated for cells using SSC-A versus FSC-A, then gated for singlets using FSC-H versus FSC-A. Subsequent fluorescence gating was used to analyze specific cell populations as needed; for example, gating on miRFP720+ cells to analyze only COMET reporter cells. In all experiments, fluorescent data were compensated for spectral bleed-through using HEK293FT cells transfected with single color controls. To convert relative units of fluorescence to absolute fluorescence units (i.e., mean molecules equivalent of phycoerythrin-texas red (PE-TR) (MEPTRS) for SynTF delivery experiments), Spherotech 9-peak rainbow beads (URCP-100-2H) were diluted ~20× in PBS and run in parallel for each experiment. Calibration curves were generated using the fluorescence intensities from the flow cytometer and the number of fluorophores per bead population as provided by the supplier. The calibration curve was then used to convert mean fluorescence intensities of cells to MEPTRs (FIG. 21C).

TABLE 7
BD Fortessa flow cytometry lasers and photomultiplier tube (PMT)
filter sets for specific fluorophores evaluated in this study.

| | Channel | Excitation | |
|---|---|---|---|
| Fluorophore | name | laser (nm) | Filter set* |
| eBFP2 | Pacific Blue | 405 | 450/50 |
| mTAGBFP2 | Pacific Blue | 405 | 450/50 |
| dsRedExpress2 | PE-Texas Red | 552 | 610/20; 600LP |
| miRFP720 | Alexa 750 | 685 | 730/45; 690 LP |

*LP, Long pass

Data Analysis/Statistical Analysis: Data was analyzed using Graph Pad Prism 9. Error bars generally represent the standard error of the mean (SEM); exceptions can be found in the relevant figure captions. Statistical tests were performed in Prism 9; details can be found in the relevant figure captions.

### Example 2: Extracellular Vesicles are Lipid-Raft Like in Membrane Physical Features and Protein Composition

The present example demonstrates, among other things, that extracellular vesicles are lipid-raft like in membrane physical features and protein composition.

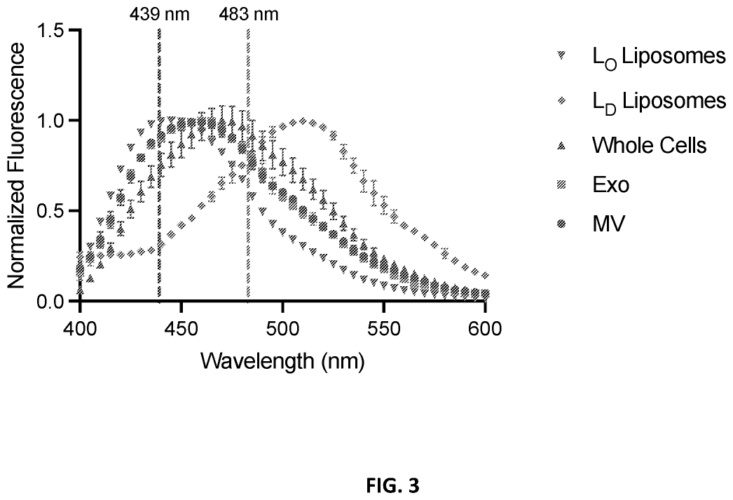

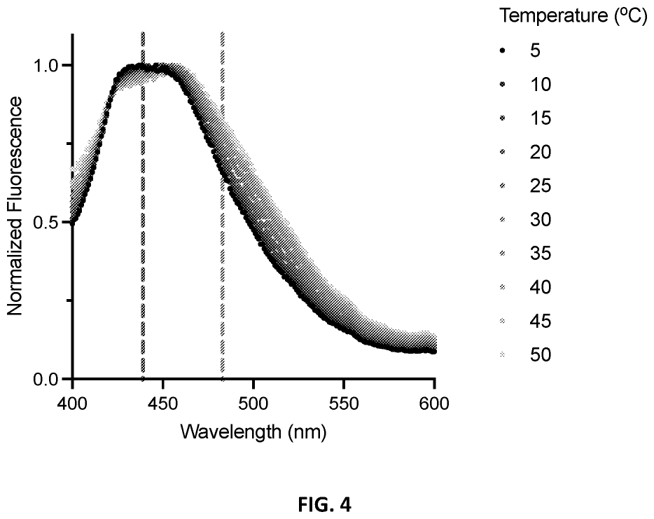

To determine whether biomolecular cargo can be loaded into EVs based on polypeptide association with lipid-rafts, the physical properties of HEK293FT-derived EV membranes was characterized. The lipid packing of EV membranes was measured via spectroscopy of the environment-sensitive dye, C-laurdan. The spectra of C-laurdan becomes blue-shifted in more ordered, less aqueous environments, which enables membrane order to be quantified. Two subpopulations of EVs from HEK293FTs were harvested via differential ultracentrifugation (FIG. 1A). The population pelleted at 15,000 g are microvesicles (MV) and the population pelleted at 120,416 g are exosomes (Exo). These populations had the expected 100-200 nm size, were enriched in classic EV markers CD81, CD9, and Alix, and were depleted in the non EV-associated endoplasmic reticulum protein, calnexin (FIG. 1B, 1C). The membrane order of microvesicles and exosomes were measured and compared to the membrane order of raft-like, “liquid-ordered” synthetic liposomes (L_{O}, composed of 70 mol % DPPC/30 mol % Chol,), and non-raft-like “liquid-disordered” liposomes (L_{D}, composed of 70 mol % DOPC/30 mol % Chol), and whole HEK293FT cells (FIG. 2C, FIG. 3, FIG. 4). It was determined that microvesicles and exosomes were each more ordered than whole cells, and both were similarly ordered to raft-mimetic liposomes, suggesting that both populations of EVs are raft-like, demonstrating that EV membranes are highly ordered.

As EV membranes were determined to be lipid-raft like in lipid order, the proteins reported to associate with EVs were analyzed using bioinformatics to evaluate whether a relationship exists between protein raft association and EV loading. Specifically, whether lipid-raft associated proteins are more likely to be found in EVs than a group of randomly selected membrane-associated proteins was evaluated. Three protein databases were utilized for this analysis: Exocarta, an EV protein database, RaftProt, a raft protein database, and Swiss-Prot, a general annotated database of human proteins. First, the frequency with which 100 randomly selected, human membrane-associated proteins from the Swiss-Prot database appear in EVs was calculated. This was then repeated for raft associated proteins (FIG. 2D). Interestingly, a majority of proteins sampled from the raft protein database were also found in the EV database, and this representation was greater relative to the randomly selected membrane-associated proteins sampled from the Swiss-Prot database, suggesting that proteins which are known to associate with lipid rafts are more frequently loaded into EVs.

### Example 3: Proteins Found in Extracellular Vesicles Exhibit Physical Features that are Known to Aid in Protein Raft Association

The present example demonstrates, among other things, that proteins found in extracellular vesicles exhibit physical features that are known to aid in protein raft association.

Without wishing to be bound by any one theory, it was hypothesized that, by characterizing the properties of proteins which natively associate with lipid rafts, proteins that efficiency load into EVs could be rationally engineered. To identify properties common to proteins found in lipid rafts and extracellular vesicles, lists of human proteins predicted to associate with lipid rafts and reported to associated with EVs were compiled using Raftprot 2.0 and Exocarta, respectively. Next, the protein biophysical and chemical information was integrated into the lists of raft and EV proteins using an annotated human protein database (Swiss-Prot), producing a new database. The new database enabled comparison of protein structural features of proteins found in lipid rafts and EVs to all membrane-associated proteins (Table 8). Without wishing to be bound by any one theory, it was hypothesized that the structural features that are enriched in known raft- or EV-associated proteins, relative to the average for a human protein, likely aid in protein association with raft and EV membranes. Specifically, the following properties were examined: the presence of a defined transmembrane domain; transmembrane domain length and number; and posttranslational modifications such as lipidation, glycosylation, and phosphorylation. These properties were chosen as they affect protein-membrane interactions, cellular trafficking, and lipid raft localization.

TABLE 8
Number of proteins analyzed in bioinformatic
study for each protein category.

| | | Single-pass | Multi-pass | Peripheral |
|---|---|---|---|---|
| | | transmembrane | transmembrane | membrane |
| | Protein | protein | protein | protein |
| Raft | Plasma | 81 | 46 | 122 |
| | membrane | | | |
| | Internal | 149 | 82 | 141 |
| | membrane | | | |
| EV | Plasma | 353 | 274 | 541 |
| | membrane | | | |
| | Internal | 370 | 272 | 540 |
| | membrane | | | |
| All | Plasma | 904 | 1463 | 1329 |
| Human | membrane | | | |
| Proteins | Internal | 1463 | 1360 | 1235 |
| | membrane | | | |

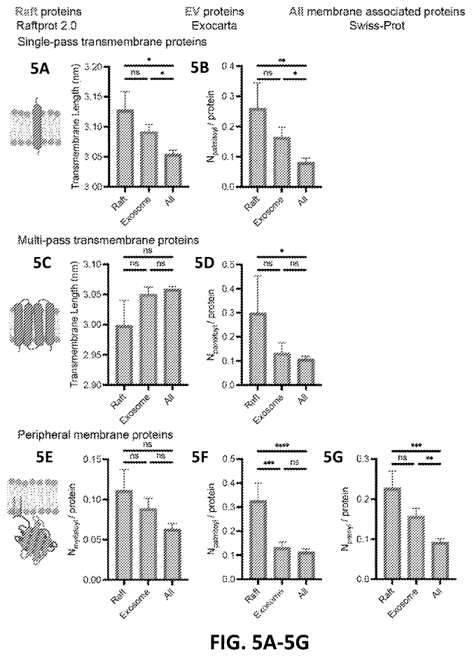

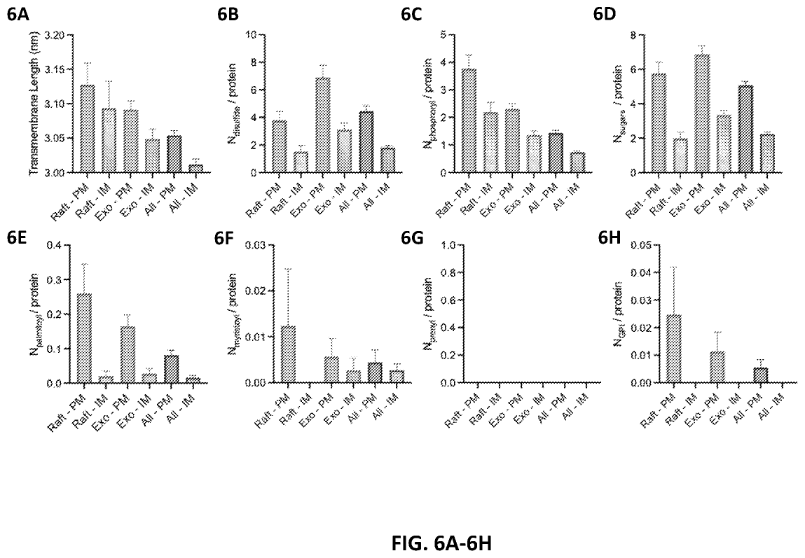

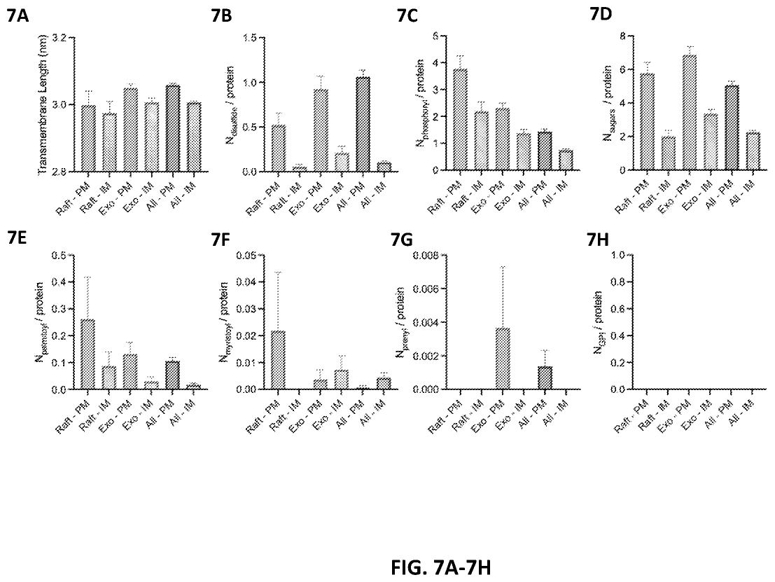

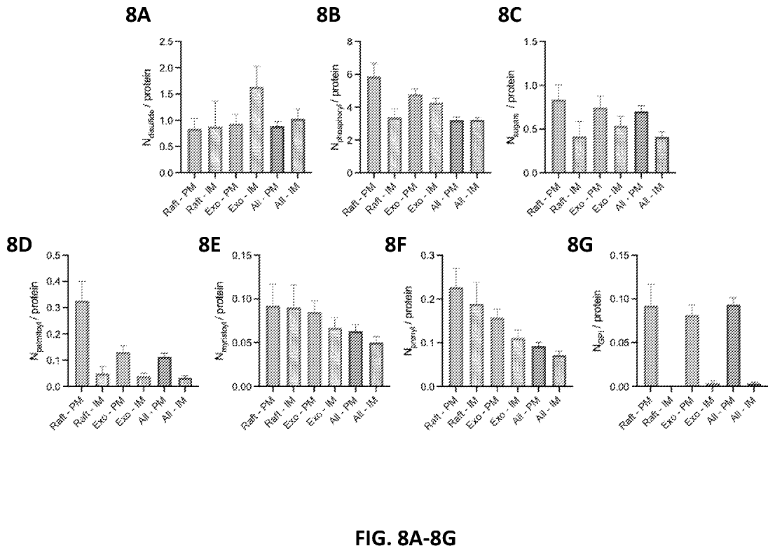

Such analyses were performed for single-pass, multi-pass, and peripheral membrane proteins. It was determined that lipid raft- and EV-associated proteins possessed many similar physical properties and modifications (FIG. 5A-5G, FIG. 6A-6H, FIG. 7A-7H, FIG. 8A-8G). To determine which protein features are most likely to enhance protein raft association and EV loading, the search was confined to proteins that localized to the plasma membrane, because the plasma membrane is highly ordered and similar in composition to EV membranes (FIG. 5A, 5C, 5E). It was determined that lipid raft- and EV-associated single-pass transmembrane proteins possessed significantly longer average transmembrane domain lengths and higher palmitoylation number compared to the pool of all single-pass transmembrane proteins (FIG. 5A, 5B). Interestingly, for multi-pass transmembrane proteins, transmembrane domain length was not significantly different between the groups of proteins, but palmitoylation appeared to remain a differentiating feature between raft- and EV-associated proteins relative to random proteins from the plasma membrane (FIG. 5C, 5D). Peripheral membrane proteins that associated with rafts and EVs were further found to possess specific lipidation groups, particularly palmitoyl, and prenyl groups, at a greater than average rate (FIG. 5E-5G). This analysis demonstrates that raft- and EV-associated proteins often possess similar properties and chemical features, further supporting that EVs are enriched in raft-associated proteins (FIG. 2A-2D) and providing a blueprint for modifying proteins to enhance loading into EVs.

### Example 4: Modulating Lipid Raft Affinity of Proteins Affects Cellular Trafficking and Loading into Extracellular Vesicles

The present example demonstrates, among other things, that modulating lipid raft affinity of proteins affects cellular trafficking and loading into extracellular vesicles.

The ability of raft associating proteins to load into extracellular vesicles was evaluated by genetically adding lipid tags to biomolecular cargo. A library of fluorophore-tagged fusion proteins were produced to experimentally characterize how modulating lipid-protein interactions affects protein loading into EVs. Based on the bioinformation analysis presented herein, transmembrane proteins were designed in which transmembrane domains varied in length, number of passes, geometry, and lipidation state. Similarly, soluble proteins were designed with different types and amounts of lipidation. Each protein was tagged with a fluorescent moiety—either a monomeric red fluorescent protein (mRFP1) or a HaloTag labeled with a tetramethyl rhodamine (TMR) dye. These fluorescent molecules allowed for evaluation of protein trafficking in live cells, lipid-raft association in giant plasma membrane vesicles (GPMVs), and loading into EVs (FIG. 9A).

Linker for Activation of T-cells (LAT) was selected as a model transmembrane protein to evaluate how protein modifications affect lipid-protein interactions, protein trafficking, and EV loading. LAT naturally associates with lipid rafts and the plasma membrane, making it a useful model protein for this study. Fusion polypeptides comprising a HaloTag and a biomolecular cargo selected from: LAT with its palmitoylated cysteine mutated to a non-palmitoylated alanine (C26A), LAT with the middle six amino acids removed from its transmembrane domain (dCore), and LAT with all transmembrane alanines replaced with leucines to increase the transmembrane surface area (High ASA) were generated. It was hypothesized that by removing a palmitoylation site, shortening the transmembrane domain, or increasing transmembrane surface area, LAT should (i) decrease its association with the plasma membrane and lipid rafts and (ii) decrease its loading into EVs relative to wild-type LAT (WT).

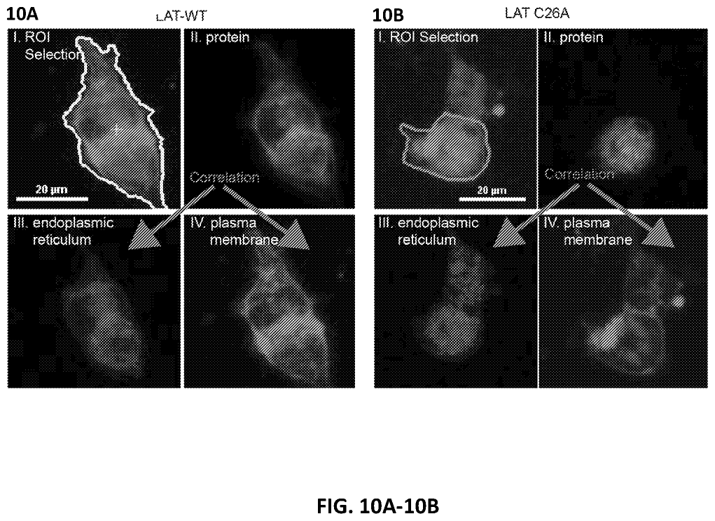

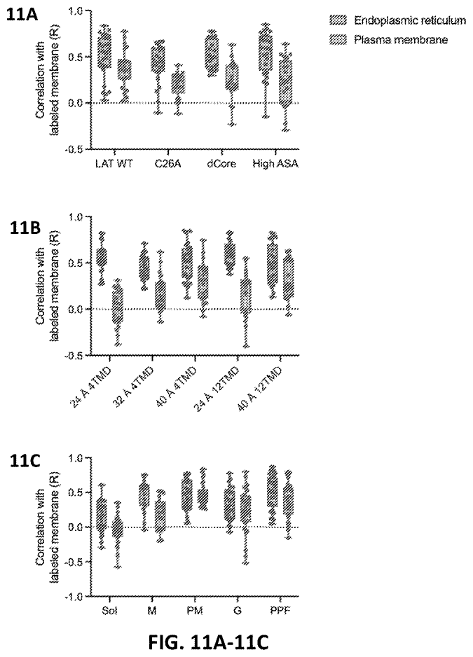

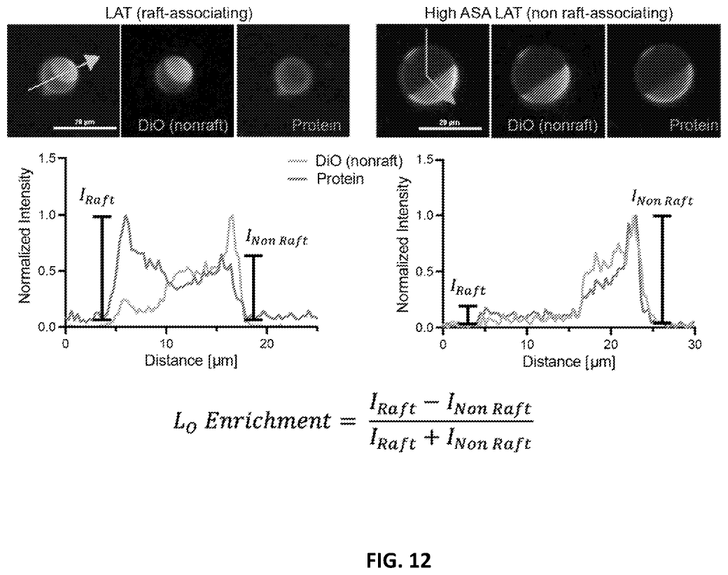

Next, the impact of LAT modifications on protein localization to the plasma membrane and lipid rafts were evaluated. Colocalization of protein and membrane-associated dyes in live cells and giant plasma membrane vesicles (GPMVs), respectively, were measured. First, HEK293FT cells were transfected with each of these constructs and confocal microscopy with live cells was used to evaluate the covariance across the image between the protein and either a plasma membrane dye or endoplasmic reticulum dye (calculated using Pearson's coefficients) (FIG. 9B, FIG. 10A-10B). It was determined that wild-type LAT preferentially localized to the plasma membrane more so than did the mutants (FIG. 9C, FIG. 11A). To explore how LAT modification impacts association with lipid rafts, LAT localization in GPMVs—vesicles derived from cellular plasma membranes that are often used to study protein association with lipid rafts—was evaluated. HEK293FT cells were transfected with each construct and generated GPMVs through the addition of vesiculation agent (paraformaldehyde and dithiothreitol) such that the plasma membrane blebs off from the cell to create protein-incorporated giant vesicles. Protein localization to lipid rafts was visualized by labeling GPMVs with DiO—a fluorescent membrane dye which localizes to liquid disordered lipid domains (i.e., non-raft regions)—and cooling vesicles below their miscibility temperature (FIG. 9D, FIG. 12). Wild-type LAT partitioned into lipid rafts more so than did the LAT mutants (FIG. 9E). Surprisingly, a raft-partitioning value could not be measured for LAT dCore because this protein did not localize to the plasma membrane and accordingly did not end up in GPMV membranes. This result agrees with cellular trafficking data in FIG. 9C. However, these results might suggest that membrane-protein interactions are cell-type specific, without wishing to be bound by any one theory, perhaps due to interaction with other species not explicitly evaluated here.

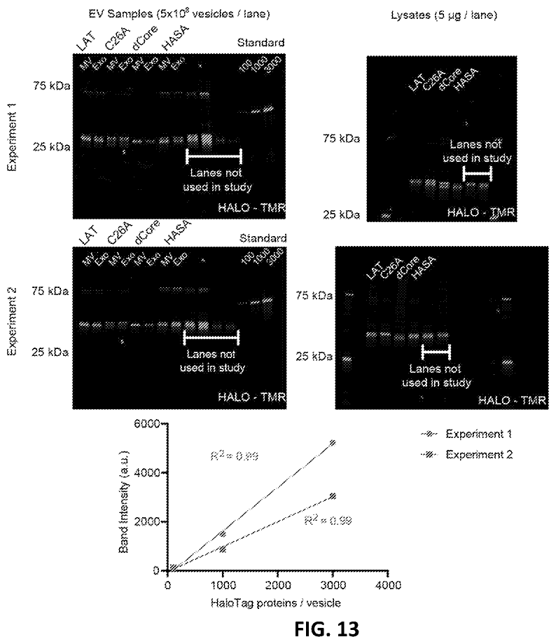

Once it was established that removing a palmitoylation site, shortening the transmembrane domain, and increasing transmembrane domain hydrophobic surface area reduced LAT localization to the plasma membrane and association with lipid rafts, how these changes impacted EV loading were characterized. Each construct was transfected into HEK293FT cells and microvesicles and exosomes were harvested via differential ultracentrifugation. Equal numbers of vesicles were processed via SDS-PAGE, and protein loading into each vesicle population was quantified by probing for the fluorescent TMR-labeled HaloTag on each LAT construct. Notably, wild-type LAT loaded into both microvesicles and exosomes to a greater extent than did most LAT mutants (FIG. 9F, FIG. 13). Protein loading for the C26A and dCore mutants was less than observed for wild-type LAT, which is consistent with the plasma membrane localization and raft partitioning experiments (FIG. 9C, 9E, FIG. 11A-11B). Interestingly, the high surface area LAT mutant loaded into EVs nearly as well as did wild-type LAT. However, the high surface area LAT construct also accumulated in cells to higher levels than did wild-type LAT (FIG. 13). To account for this expression difference, protein loading in EVs was normalized by total protein expression in cells. This ratio is referred to herein as, ‘EV partitioning’ (FIG. 9G). Wild-type LAT possessed the highest EV partitioning value. This metric reflects the trends observed for plasma membrane localization and raft partitioning and portrays the effect of lipid-protein interactions on EV loading, independent of effects upon overall protein expression/accumulation. Altogether, WT LAT, which most strongly associated with the plasma membrane and lipid rafts, also loaded most effectively into EVs. Mutations which reduce association with the plasma membrane and lipid rafts, such as the removal of a palmitoylation site or shortening a transmembrane domain, reduced EV loading. These data are reflective of the computational analysis in FIG. 5A-5G—longer, palmitoylated single-pass transmembrane domains localize more strongly to EV membranes than shorter, non-palmitoylated proteins—suggesting that such principles may be applied to an array of single pass transmembrane proteins.

Next, whether the trends identified for LAT proteins would hold for different, unrelated transmembrane proteins was evaluated. To accomplish this, two sets of de novo designed transmembrane protein complexes—a transmembrane hairpin dimer (4TMD, four transmembrane domains) and a hexameric transmembrane hairpin design (12TMD, twelve transmembrane domains)—which vary in transmembrane domain length from 24 to 40 Å (FIG. 14A) were utilized. Without wishing to be bound by any one theory, it was reasoned that because these polypeptides are completely synthetic, it was hypothesized that they would be trafficked and loaded into EVs based primarily on physical interactions with membranes, rather than through evolved protein-protein interactions and trafficking mechanisms. Fusion polypeptides comprising C-terminal mRFP1 and each de novo designed protein were generated and how the design choices sampled influenced protein trafficking to the plasma membrane and association with lipid rafts was evaluated. In live cells, for both protein designs, increasing transmembrane domain length increased plasma membrane localization (FIG. 14B, FIG. 11B). This agreed with the characterization of LAT proteins. In GPMV assays, none of the constructs appeared to localize to lipid rafts (FIG. 14C). The computational analysis in FIG. 5A-5G showed that lipidation was a strong determinant of lipid raft association, particularly for multi-pass transmembrane proteins. Thus, it is reasonable that these non-lipidated proteins do not associate with lipid rafts. Like the shortened dCore LAT construct, both 24 Å de novo designed transmembrane domains exhibited reduced localization to GPMV membranes relative to the longer 32 Å, and 40 Å constructs, likely because the former do not traffic well to the plasma membrane.

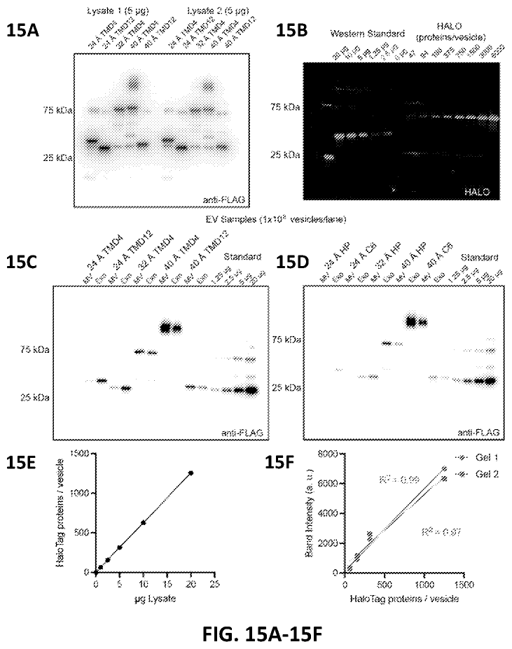

Additionally, loading and partitioning of each set of de novo designed transmembrane proteins into EVs was characterized. Transmembrane domain length positively correlated with EV loading and EV partitioning for the proteins with four transmembrane domains (FIG. 14D, 14E, FIG. 15A-15F). However, this trend was not observed for the hexameric transmembrane hairpin designs (12TMD). This outcome is consistent with our bioinformatic data, as transmembrane domain length did not correlate with raft or EV association for multi-pass transmembrane proteins (FIG. 5C). Interesting, we found that both 40 Å protein (40 Å 4TMD and 40 Å 12TMD) constructs loaded much more effectively into MVs compared to exosomes relative to their 24 Å counterparts. This suggests that lipid-protein interactions may be harnessed to not only increase protein loading into EVs, but that such effects may drive loading into distinct vesicle populations. Combined, the LAT and de novo designed protein data indicate that protein trafficking to the plasma membrane and lipid raft association increase protein loading into EVs.

Next, how protein engineering to modulate membrane association can be applied to load soluble, cytosolic proteins into EVs was assessed. A link between EV loading, cellular trafficking, and raft association for lipidated, peripheral membrane proteins has not yet been established. Using data compiled in the initial bioinformatic analysis, protein sequences were selected that appeared in either the N- or C-terminal regions of proteins and were natively lipidated. Specifically, protein sequences known to be modified different amounts and types of lipid moieties (e.g., myristoyl, palmitoyl, farnesyl, geranylgeranyl) were chosed. Five fusion polypeptides comprising HaloTag-containing proteins were generated: unlipidated, cytosolic/soluble HaloTag (Sol); a HaloTag with a single N-terminal myristylation site (M); a HaloTag with an N-terminal palmitoylation and myristylation site (PM); a HaloTag with a single C-terminal geranylgeranylation site (G); and a HaloTag with two palmitoylation sites and a single farnesylation site on the C-terminus (PPF) (FIG. 16A). Lipid tags were placed on termini in a manner that matched the native location of the tags (i.e., within the source protein on which the tag is modeled). Plasmids encoding each construct were transfected into HEK293FT cells and how each protein localized to the endoplasmic reticulum and plasma membrane was evaluated (FIG. 16B, FIG. 11C). Soluble, non-lipidated HaloTag did not localize to the plasma membrane. Further, it appeared that a single lipidation site was sufficient to increase association of cargo protein with the plasma membrane (M, G); the addition of multiple lipid sites strengthened the association with the plasma membrane (PM, PPF). It was then investigated how each of these constructs associated with lipid rafts in GPMVs (FIG. 16C). The PM and PPF tags conferred more association with lipid-rafts than did the M and G tags. In contrast, soluble HaloTag was observed inside GPMVs but did not associate with GPMV membranes.

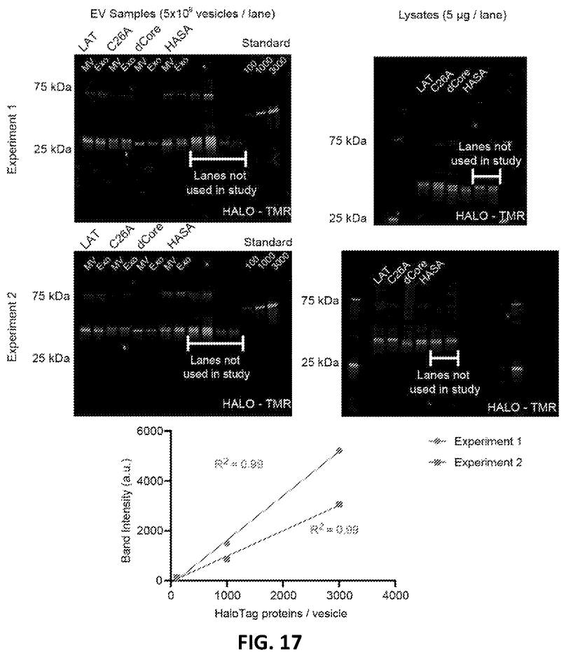

Next, how each protein loaded and partitioned into EVs was characterized. HEK293FT cells were transfected with each construct, EVs were harvested, and protein loading and partitioning into EVs was determined via SDS-PAGE. Lipidation increased HaloTag loading relative to soluble HaloTag for all conditions in both EV populations, achieving up to 400 proteins per vesicle and a loading improvement of up to ~4× over passive loading (FIG. 16D, FIG. 17). When normalizing to overall protein expression in the producer cells, it was determined that the motifs which strongly associated with lipid rafts (i.e., PM, PPF) generally drove the greatest loading of cargo into EVs, achieving a 5-10× increase in EV partitioning relative to soluble HaloTag (FIG. 16E). Although the G tag did not drive association with lipid rafts, it did confer high EV partitioning. This result suggested that although lipid raft association can drive protein loading into EVs, it is not required. Interestingly, it was observed that when cells expressed protein constructs that strongly partition to the plasma membrane and into EVs, as much as 50% more microvesicles, but not exosomes, were generated; this effect correlates with the degree of EV partitioning (FIG. 16F-16H). This protein-induced microvesicle formation could occur due to increased membrane budding as a result of protein crowing on the plasma membrane and suggests a possible strategy to intentionally increase EV production. Taken together, these data suggest a positive correlation between plasma membrane and lipid raft association and EV loading for cytosolic protein cargo, further validating bioinformatic analysis presented in FIG. 5A-5G.

Taken together, the data presented in FIG. 9A-9G, FIG. 14A-14E, and FIG. 16A-16H provide insight into how lipid-protein interactions affect protein trafficking and ultimately loading into EVs. In addition, how MV and Exo loading and EV partitioning correlate with the cellular trafficking, lipid interaction, and EV property data was systematically evaluated (FIG. 9A-9G, FIG. 14A-14E, FIG. 16A-16H, ands Tables 9 and 10).

TABLE 9
Number of EVs produced from HEK293FTs transfected for all constructs
in this study, as determined by nanoparticle tracking analysis

| | MVs | MV | Exos | Exo |
|---|---|---|---|---|
| Construct | produced | Std | Produced | Std |
| Sol | 8.26E+09 | 1.81E+09 | 1.02E+10 | 2.21E+09 |
| M | 9.32E+09 | 1.99E+09 | 1.08E+10 | 2.35E+09 |
| PM | 1.67E+10 | 3.56E+09 | 1.21E+10 | 2.63E+09 |
| GG | 1.40E+10 | 3.03E+09 | 1.34E+10 | 2.96E+09 |
| PPF | 1.53E+10 | 3.31E+09 | 7.04E+09 | 1.75E+09 |
| LAT WT | 6.97E+09 | 1.59E+09 | 8.16E+09 | 1.79E+09 |
| LAT C26A | 5.98E+09 | 1.33E+99 | 6.90E+09 | 1.55E+09 |
| LAT dCore | 5.87E+09 | 1.26E+09 | 8.74E+09 | 1.94E+09 |
| Lat High ASA | 8.01E+09 | 1.73E+09 | 8.02E+09 | 1.79E+09 |
| 24 Å TM4 | 5.91E+09 | 1.27E+09 | 1.04E+10 | 2.26E+09 |
| 32 Å TM4 | 7.09E+09 | 1.56E+09 | 8.37E+09 | 1.81E+09 |
| 40 Å TM4 | 7.06E+09 | 1.52E+09 | 1.14E+10 | 2.49E+09 |
| 24 Å TM12 | 6.95E+09 | 1.51E+09 | 1.23E+10 | 2.62E+09 |
| 40 Å TM12 | 5.54E+09 | 1.21E+09 | 1.24E+10 | 2.68E+09 |

TABLE 10
EV size for vesicles harvested from HEK293FTs that
were transfected with the below constructs, as
determined by nanoparticle tracking analysis

| | MV Size | MV Std | Exo Size | Exo Std |
|---|---|---|---|---|
| | (nm) | (nm) | (nm) | (nm) |
| Sol | 197.4 | 103.1 | 160.1 | 64.9 |
| M | 202.7 | 110.2 | 165.7 | 67.2 |
| PM | 205.7 | 100.8 | 164.5 | 72.9 |
| GG | 207.6 | 99.2 | 171.3 | 71.7 |
| PPF | 203.5 | 100.9 | 161.9 | 80.7 |
| LAT WT | 191.0 | 84.2 | 160.3 | 71.4 |
| LAT C26A | 184.6 | 96.1 | 158.6 | 72.4 |
| LAT dCore | 185.8 | 101.3 | 159.6 | 70.9 |
| Lat High ASA | 190.3 | 102.9 | 161.8 | 70.6 |
| 24 Å TM4 | 173.8 | 90.9 | 151.3 | 72.8 |
| 32 Å TM4 | 181.6 | 92.0 | 148.9 | 72.6 |
| 40 Å TM4 | 174.8 | 91.2 | 148.3 | 65.5 |
| 24 Å TM12 | 172.4 | 81.0 | 155.2 | 66.3 |
| 40 Å TM12 | 173.0 | 86.6 | 153.9 | 64.6 |

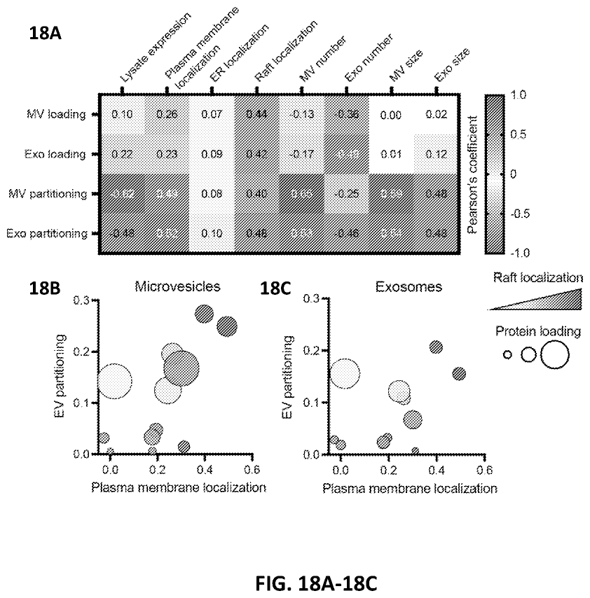

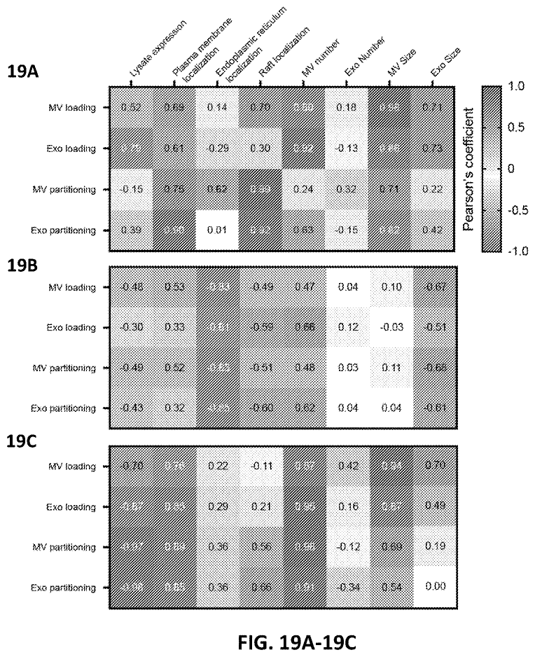

Plasma membrane localization and raft association correlated strongly with MV and exosome partitioning (FIG. 18A). The correlation between raft association and EV partitioning was even stronger for the LAT proteins and soluble HaloTag constructs when each family of proteins was analyzed separately (reaching Pearson's coefficients of up to 0.98) (FIG. 19A-19C). To further visualize protein loading trends, multivariable plots comparing plasma membrane localization, protein partitioning, raft localization, and protein loading into MVs and exosomes were generated. From these plots, it was observed that plasma membrane and raft localization are positively correlated with protein partitioning into both populations of vesicles (FIG. 18B, 18C). These observations, derived from data collected on structurally diverse proteins, strongly support the hypothesis that plasma membrane and raft localization are important parameters for protein partitioning into EVs. Further, the strong agreement between the bioinformatic analysis (FIG. 1C, FIG. 5A-5G) with EV loading data (FIG. 9A-9G, FIG. 14A-14E, and FIG. 16A-16H) indicates that this is a generalizable mechanism governing protein loading into EVs.

### Example 5: An Engineered, Lipidated Transcription Factor Loads into EVs and Drives Gene Expression after EV-Mediated Delivery to Target Cells

The present example demonstrates, among other things, that an engineered, lipidated transcription factor loads into EVs and drives gene expression after EV-mediated delivery to target cells.

Whether features that enhance lipid raft association and EV loading could be harnessed to enable engineered EV therapeutics was evaluated. While simply enhancing EV loading could lead to better protein delivery to target cells, it is also possible that the modification of protein cargo to enhance association with EVs might inhibit the functionality of the cargo or prevent its release from the EV membrane upon membrane fusion with the host cell. Therefore, how modification of a biomolecular cargo (e.g. by lipid tag) to enhance its loading into EVs affected its functional delivery and release (into the cytoplasm) in recipient cells was evaluated.

The EV-mediated, functional delivery of a synthetic transcription factor (SynTF) as a model system was utilized. Specifically, the COmposable Mammalian Elements of Transcription (COMET) toolkit that comprises a set of SynTFs and cognate promoters was used. Lipid tags were fused to a SynTF, that consists of a zinc-finger DNA binding domain, a p65 transcriptional activator domain, and a nuclear localization sequence, to generate a fusion polypeptide. Then, cargo loading and functional delivery to a HEEK293FT-based reporter cell line was assessed (FIG. 20A, FIG. 21A-21C). This reporter line contains a genetically integrated expression cassette that includes the cognate promoter for the selected SynTF upstream of a dsRedExpress2 reporter gene; functional delivery of a SynTF is indicated by induction of dsRedExpress2 fluorescence. In addition, the expression cassette contains a constitutive miRFP720 fluorescent protein to identify cells with an active reporter construct.

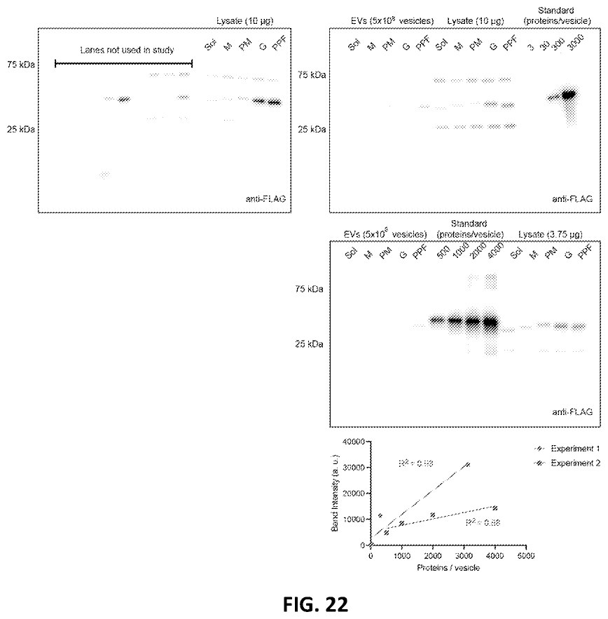

To determine if lipidated SynTFs were capable of driving reporter expression, first SynTF-encoding plasmids were directly transfected into reporter cells. The addition of lipid tags to the transcription factor impaired the ability to induce reporter expression to different extents across the constructs (compared to the non-lipidated “Sol” SynTF control), although all transcription factors were still functional to some degree (FIG. 20B). Differences in SynTF-induced reporter expression were not attributable to differential expression of the SynTFs, suggesting that lipidation modulates specific (per protein) functional activity of the SynTFs (FIG. 20B, FIG. 22). Next, EVs were harvested from cells transfected with SynTF plasmids and it was determined that the more raft associated tags, PM and PPF (FIG. 16C), led to the highest loading of SynTFs into EVs (FIG. 20C, FIG. 22). Most notably, the PM and PPF tags increased EV loading relative to the soluble SynTF by 15× and 52×, respectively. Taking the EV-loading data from lipidated HaloTag and SynTF together, it was concluded that lipid raft association is an EV-loading strategy that is robust and agnostic to biomolecular cargo identity. This contrasts with strategies for enhancing loading that are not raft-dependent; non-raft tags such as G conferred loading of the HaloTag construct but not the SynTF construct (FIG. 16D-16E, FIG. 20C).

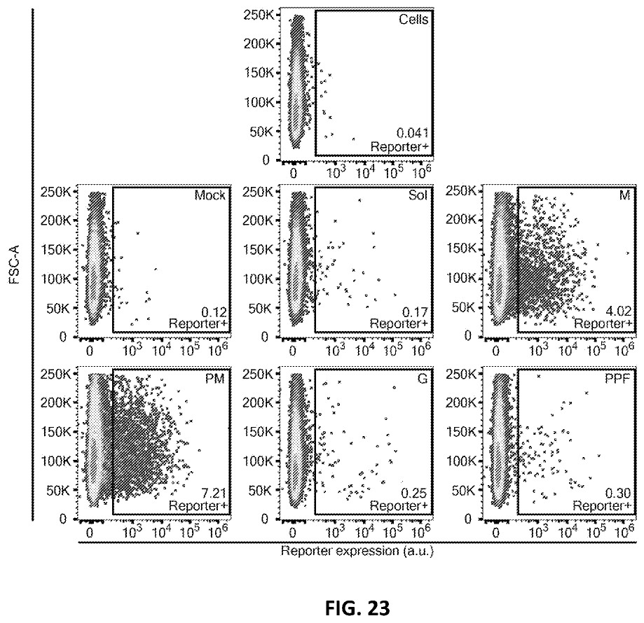

Finally, whether EV-mediated delivery of lipid-modified SynTFs could drive functional changes in recipient cells was evaluated. Reporter cells were treated with EVs containing lipidated ZFs (fusion polypeptides comprising a lipid tag and a ZF as biomolecular cargo) and a surface displayed viral glycoprotein (vesicular stomatitis virus G protein, VSV-G, without a lipid tag) to promote EV uptake and membrane fusion with recipient endosomal membranes. Notably, EV-mediated delivery of several lipidated SynTF designs successfully induced reporter expression, while EV-mediated delivery of SynTF (Sol) conferred no substantial reporter induction (FIG. 20D-20F, FIG. 23). The PM-modified SynTF partitioned strongly to lipid rafts (FIG. 16C) and converted up to ~7% of reporter cells to a substantially activated state (FIG. 20F). These observations highlight the utility and tradeoffs associated with using a lipid-raft associating tag such as PM: despite having <10% of the specific (per protein) transcriptional activity relative to a soluble SynTF (FIG. 20B), the PM tag enabled EV-mediated delivery of SynTF cargo to an extent that far exceeded the soluble SynTF. Without wishing to be bound by any one theory, it is hypothesized that discrepancies between EV loading and functional delivery for some lipid tags (i.e. PPF's high loading but low functional delivery) may be due to a differential ability of the tags to dissociate from the EV membrane after delivery and membrane fusion with reporter cells. Presumably, lipid tags that enable association during EV generation but disassociation after delivery into target cells should yield the most effective modifications for TF delivery. It is also likely that tags which drive EV loading may lead to a loss in apparent specific activity in a high SynTF expression assay (e.g., FIG. 20B), but this effect is countered by the benefits of active loading and/or may not diminish overall function if the SynTF

1. A method of loading an engineered vesicle (EngV) with a fusion polypeptide, comprising:
contacting a cell with a nucleic acid encoding a fusion polypeptide comprising (i) a biomolecular cargo, and (ii) one or more lipid tags,
wherein contacting occurs under conditions sufficient to permit (i) uptake of the nucleic acid by the cell, (ii) translation of the fusion polypeptide, and (iii) post-translational modification of the one or more lipid tags with one or more lipids by endogenous host machinery in the cell to thereby generate a lipid-modified fusion polypeptide, and wherein the lipid-modified fusion polypeptide is directed and loaded into or on the EngV.

2. The method of claim 1, wherein the one or more lipid tags comprise a short amino acid sequence which a host cell can post-translationally modify with one or more lipids.

3. The method of claim 1, wherein the one or more lipid tags comprises the amino acid sequence of any one of SEQ ID NOs: 1-13.

4. The method of claim 1, wherein post-translational modification of the one or more lipid tags with a lipid comprises one or more of acylation or prenylation.

5.-14. (canceled)

15. The method of claim 1, wherein the biomolecular cargo is or comprises a therapeutic polypeptide.

16. The method of claim 15, wherein the therapeutic polypeptide is a transcription factor.

17. The method of claim 1, further comprising isolating the loaded EngV.

18. The method of claim 1, further comprising characterizing the loaded EngV for one or more of yield, purity, loading, functional activity, or any combination thereof.

19. The method of claim 1, wherein the biomolecular cargo comprises a transmembrane domain and the transmembrane domain is modified to enhance direction and loading into or on the EngV relative to the biomolecular cargo with an unmodified transmembrane domain.

20. The method of claim 19, wherein the transmembrane domain is modified by one or more of: changing the length of the transmembrane domain and changing the transmembrane domain surface area.

21. A loaded EngV or a plurality thereof prepared by the method of claim 1.

22. A composition comprising the loaded EngV or a plurality thereof of claim 21.

23. (canceled)

24. A method of treating a disease or a disorder in a subject in need thereof comprising administering the loaded EngV or a plurality thereof of claim 21 to the subject in need thereof.

25. (canceled)

26. A fusion polypeptide comprising (i) a biomolecular cargo and (ii) one or more lipid tags, wherein the one or more lipid tags comprise a short amino acid sequence which a host cell can post-translationally modify with one or more lipids.

27. A method of loading an engineered vesicle (EV) with a biomolecular cargo, comprising: contacting a cell with a nucleic acid encoding the biomolecular cargo, wherein contacting occurs under conditions sufficient to permit (i) uptake of the nucleic acid by the cell, (ii) translation of the biomolecular cargo,
wherein the biomolecular cargo comprises a transmembrane domain and the transmembrane domain is modified to improve direction and loading into or on the EngV relative to the biomolecular cargo with an unmodified transmembrane domain, thereby generating a transmembrane modified biomolecular cargo, and
wherein the transmembrane modified biomolecular cargo is directed and loaded into or on the EngV.

28.-29. (canceled)

30. A loaded EngV or a plurality thereof prepared by the method of claim 27.

31. A composition comprising the loaded EngV or a plurality thereof of claim 30.

32. The composition of claim 31, wherein the composition is a pharmaceutical composition.

33. A method of treating a disease or a disorder in a subject in need thereof comprising administering the loaded EngV or a plurality thereof of claim 30 to the subject in need thereof.

34. A nucleic acid encoding the transmembrane modified biomolecular cargo of claim 1.

35. (canceled)
